(19) United States
(12) Patent Application Publication
(10) Pub. No.: US 2015/0126736 A1
(43) Pub. Date: May 7, 2015

(54) NOVEL ORGANIC ELECTROLUMINESCENCE COMPOUNDS AND ORGANIC ELECTROLUMINESCENCE DEVICE CONTAINING THE SAME

(71) Applicant: Rohm and Haas Electronic Materials Korea Ltd., Cheonan (KR)

(72) Inventors: Young-Jun Cho, Seongnam (KR); Ji-Song Jun, Hwaseong (KR); Hee-Ryong Kang, Seoul (KR); Bong-Ok Kim, Seoul (KR); Hyuck-Joo Kwon, Seoul (KR); Kyung-Joo Lee, Seoul (KR); Seon-Woo Lee, Osan (KR); Hong-Yeop Na, Seoul (KR); Hyo-Nim Shin, Seongnam (KR); Jeong-eun Yang, Suwon (KR); Soo-Jin Yang, Hwaseong (KR)

(21) Appl. No.: 14/398,227

(22) PCT Filed: May 2, 2013
(86) PCT No.: PCT/KR2013/003813

§ 371 Date: Oct. 31, 2014

(30) Foreign Application Priority Data
May 2, 2012 (KR) 10-2012-0046247

(51) Int. Cl. H01L 51/00; C07D 403/14

(52) U.S. Cl. CPC H01L 51/0067; C07D 403/14; H01L 51/0072; H01L 51/5012
USPC 544/212; 544/331; 544/180

(57) ABSTRACT

The present invention relates to a novel organic electroluminescent compound and an organic electroluminescent device comprising the same. The organic electroluminescent compound according to the present invention has excellent luminous efficiency, power efficiency, and lifespan characteristic. Using the compounds of the present invention, it is possible to manufacture an OLED device with a long operating lifespan. In addition, the compounds can improve the power efficiency of the device to reduce overall power consumption.

### TECHNICAL FIELD

The present invention relates to novel organic electroluminescent compounds and organic electroluminescent device containing the same.

### BACKGROUND ART

An electroluminescent (EL) device is a self-light-emitting device. When a charge is applied between an anode and a cathode, a hole and an electron are injected from the anode and the cathode, respectively. The hole and the electron are reunited to form an exciton. The EL device emits light corresponding to the wavelength of the energy gap that occurred from the transition of the exciton to a ground state.

The light emission is categorized as fluorescence which is the use of an exciton in a singlet state; and phosphorescence which is the use of an exciton in a triplet state. In view of quantum mechanics, phosphorescent light emitting materials enhance luminous efficiency by about four (4) times compared to fluorescent light emitting materials.

In the EL device, a luminescent dye (dopant) can be used in combination with a host material as a light emitting material to improve color purity, luminous efficiency, and stability. Since, host materials greatly influence the efficiency and the performance of the EL device when using a host material/dopant system as a light emitting material, their selection is important.

Though a conventional phosphorescent host material such as 4,4-N,N-dicarbazolebiphenyl (CBP) provides a current efficiency higher than fluorescent materials, its driving voltage is high. Thus, there are less advantages in terms of power efficiency. Further, the luminous efficiency and operating lifespan of the device still need improvement.

Korean patent publication No. 0948700 discloses an organic electroluminescent compound in which an aryl carbazole structure is substituted with a nitrogen containing heteroaryl; and an OLED comprising the compound.

However, the above prior art reference does not directly disclose a compound in which a compound wherein a 9H-carbazolyl group is substituted at the 3-position of a carbazole structure, and a substituted or unsubstituted heteroaryl is directly or indirectly substituted at the 9-position of a carbazole structure. Moreover, the compounds disclosed in the above reference still need improvement in aspects of their luminous efficiency, lifespan characteristic, and driving voltage.

### DISCLOSURE OF THE INVENTION

### Problems to be Solved

The present invention is accomplished to fulfill the above needs in the field. The objective of the present invention is first, to provide an organic electroluminescent compound imparting low driving voltage, high luminous and power efficiency, and a long lifespan to a device; and second, to provide an organic electroluminescent device of high efficiency and long lifespan, comprising the organic electroluminescent compound.

The present inventors found that the objective above is achievable by an organic electroluminescent compound represented by the following formula 1:

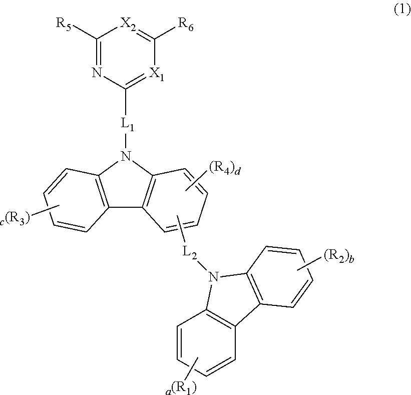

wherein

L₁ and L₂ each independently represent a single bond, a substituted or unsubstituted 5- to 30-membered heteroarylene, or a substituted or unsubstituted (C6-C30)arylene;

X₁ and X₂ each independently represent CR₇ or N;

R₁ to R₄ and R₇ each independently represent hydrogen, deuterium, a halogen, a cyano, a carboxyl, a nitro, a hydroxyl, a substituted or unsubstituted (C1-C30)alkyl, a substituted or unsubstituted (C2-C30)alkenyl, a substituted or unsubstituted (C2-C30)alkynyl, a substituted or unsubstituted (C1-C30)alkoxy, a substituted or unsubstituted (C3-C30)cycloalkyl, a substituted or unsubstituted (C3-C30)cycloalkenyl, a substituted or unsubstituted 3- to 7-membered heterocycloalkyl, a substituted or unsubstituted (C6-C30)aryl, a substituted or unsubstituted 5- to 30-membered heteroaryl, —NR₁₁R₁₂, —SiR₁₃R₁₄R₁₅, —SR₁₆, —OR₁₇, —COR₁₈ or —B(OR₁₉)(OR₂₀); or are linked to an adjacent substituent(s) to form a mono- or polycyclic, 3- to 30-membered alicyclic or aromatic ring whose carbon atom(s) may be replaced with at least one hetero atom selected from the group consisting of nitrogen, oxygen and sulfur;

R₅ and R₆ each independently represent hydrogen, deuterium, a halogen, a substituted or unsubstituted (C1-C30)alkyl, a substituted or unsubstituted (C6-C30)aryl, a substituted or unsubstituted 5- to 30-membered heteroaryl, —NR₁₁R₁₂ or —SiR₁₃R₁₄R₁₅;

R₁₁ to R₂₀ each independently represent hydrogen, deuterium, a halogen, a cyano, a carboxyl, a nitro, a hydroxyl, a substituted or unsubstituted (C1-C30)alkyl, a substituted or unsubstituted (C2-C30)alkenyl, a substituted or unsubstituted (C2-C30)alkynyl, a substituted or unsubstituted (C1-C30)alkoxy, a substituted or unsubstituted (C3-C30)cycloalkyl, a substituted or unsubstituted (C3-C30)cycloalkenyl, a substituted or unsubstituted 3- to 7-membered heterocycloalkyl, a substituted or unsubstituted (C6-C30)aryl, or a substituted or unsubstituted 5- to 30-membered heteroaryl; or are linked to an adjacent substituent(s) to form a mono- or polycyclic, 3- to 30-membered alicyclic or aromatic ring whose carbon atom(s) may be replaced with at least one hetero atom selected from the group consisting of nitrogen, oxygen and sulfur;

a, b and c each independently represent an integer of 1 to 4; where a, b or c is an integer of 2 or more, each of R₁, each of R₂, or each of R₃ may be same or different;

d represents an integer of 1 to 3; where d is an integer of 2 or more, each of R₄ may be same or different;

the heteroarylene and heteroaryl contain at least one hetero atom selected from B, N, O, S, P(═O), Si and P; and

the heterocycloalkyl contains at least one hetero atom selected from O, S and N.

### Effects of the Invention

The organic electroluminescent compounds according to the present invention can provide high luminous efficiency and power efficiency, good lifespan characteristics, and low driving voltage. Therefore, using the compounds of the present invention, it is possible to manufacture an OLED device with high current efficiency, long operational lifespan, and low power consumption.

### EMBODIMENTS OF THE INVENTION

Hereinafter, the present invention will be described in detail. However, the following description is intended to explain the invention, and is not meant in any way to restrict the scope of the invention.

The present invention relates to an organic electroluminescent compound represented by formula 1, above, an organic electroluminescent material comprising the compound, and an organic electroluminescent device comprising the material.

Hereinafter, the organic electroluminescent compound represented by the above formula 1 will be described in detail.

Herein, “(C1-C30)alkyl” is meant to be a linear or branched alkyl having 1 to 30 carbon atoms, in which the number of carbon atoms is preferably 1 to 20, more preferably 1 to 10, and includes methyl, ethyl, n-propyl, isopropyl, n-butyl, isobutyl, tert-butyl, etc.; “(C2-C30)alkenyl” is meant to be a linear or branched alkenyl having 2 to 30 carbon atoms, in which the number of carbon atoms is preferably 2 to 20, more preferably 2 to 10, and includes vinyl, 1-propenyl, 2-propenyl, 1-butenyl, 2-butenyl, 3-butenyl, 2-methylbut-2-enyl, etc.; “(C2-C30)alkynyl” is a linear or branched alkynyl having 2 to 30 carbon atoms, in which the number of carbon atoms is preferably 2 to 20, more preferably 2 to 10, and includes ethynyl, 1-propynyl, 2-propynyl, 1-butynyl, 2-butynyl, 3-butynyl, 1-methylpent-2-ynyl, etc.; “(C1-C30)alkoxy” is a linear or branched alkoxy having 1 to 30 carbon atoms, in which the number of carbon atoms is preferably 1 to 20, more preferably 1 to 10, and includes methoxy, ethoxy, propoxy, isopropoxy, 1-ethylpropoxy, etc.; “(C3-C30)cycloalkyl” is a mono- or polycyclic hydrocarbon having 3 to 30 carbon atoms, in which the number of carbon atoms is preferably 3 to 20, more preferably 3 to 7, and includes cyclopropyl, cyclobutyl, cyclopentyl, cyclohexyl, etc.; “(C6-C30)cycloalkylene” is one formed by removing a hydrogen from cycloalkyl having 6 to 30, preferably 6 to 20, more preferably 6 or 7 carbon atoms; “3- to 7-membered heterocycloalkyl” is a cycloalkyl having at least one heteroatom selected from B, N, O, S, P(═O), Si and P, preferably O, S and N, and 3 to 7 ring backbone atoms, and includes tetrahydrofuran, pyrrolidine, thiolan, tetrahydropyran, etc.; “(C6-C30)aryl(ene)” is a monocyclic or fused ring derived from an aromatic hydrocarbon having 6 to 30 carbon atoms, in which the number of carbon atoms is preferably 6 to 20, more preferably 6 to 12, and includes phenyl, biphenyl, terphenyl, naphthyl, fluorenyl, phenanthrenyl, anthracenyl, indenyl, triphenylenyl, pyrenyl, tetracenyl, perylenyl, chrysenyl, naphthacenyl, fluoranthenyl, etc.; “5- to 30-membered heteroaryl(ene)” is an aryl group having at least one, preferably 1 to 4 heteroatom selected from the group consisting of B, N, O, S, P(═O), Si and P, and 5 to 30 ring backbone atoms; is a monocyclic ring, or a fused ring condensed with at least one benzene ring; has preferably 5 to 21, more preferably 5 to 15 ring backbone atoms; may be partially saturated; may be one formed by linking at least one heteroaryl or aryl group to a heteroaryl group via a single bond(s); and includes a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, dibenzothiophenyl, benzoimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, quinoxalinyl, carbazolyl, phenoxazinyl, phenanthridinyl, benzodioxolyl, etc. Further, “halogen” includes F, Cl, Br and I.

The organic electroluminescent compound represented by formula 1 can be represented by one selected from formulae 2 to 4:

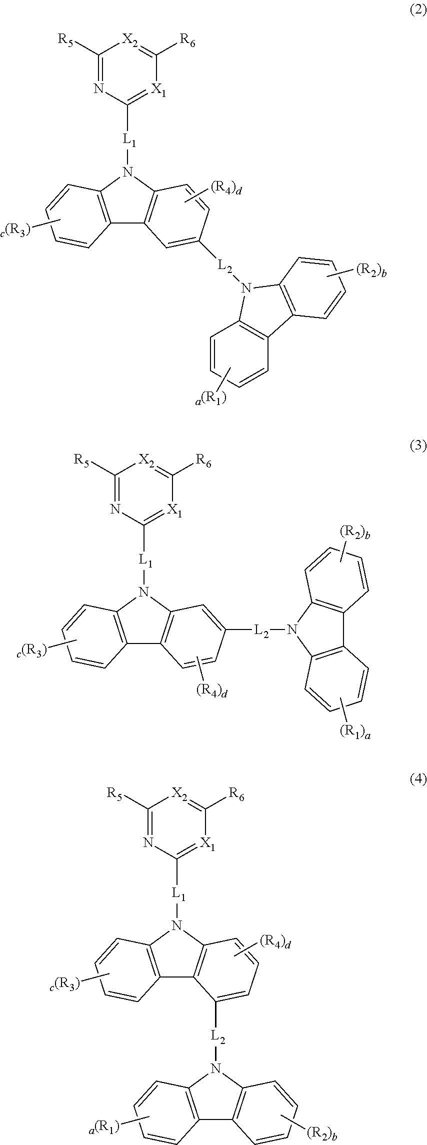

wherein L₁, L₂, X₁, X₂, R₁ to R₆, a, b, c and d are as defined in formula 1.

Herein, “substituted” in the expression “substituted or unsubstituted” means that a hydrogen atom in a certain functional group is replaced with another atom or group, i.e., a substituent.

The substituents of the substituted (C1-C30)alkyl, the substituted (C2-C30)alkenyl, the substituted (C2-C30)alkynyl, the substituted (C1-C30)alkoxy, the substituted (C3-C30)cycloalkyl, the substituted (C3-C30)cycloalkenyl, the substituted 3- to 7-membered heterocycloalkyl, the substituted (C6-C30)aryl(ene), and the substituted 5- to 30-membered heteroaryl(ene) in L₁, L₂, R₁ to R₇, R₁₁ to R₂₀ each independently are at least one selected from the group consisting of deuterium; a halogen; a cyano; a carboxyl; a nitro; a hydroxyl; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a 3- to 7-membered heterocycloalkyl; a (C6-C30)aryloxy; a (C6-C30)arylthio; a 5- to 30-membered heteroaryl unsubstituted or substituted with a (C6-C30)aryl; a (C6-C30)aryl unsubstituted or substituted with a 5- to 30-membered heteroaryl; a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di-(C1-C30)alkylamino; a mono- or di-(C6-C30)arylamino; a (C1-C30)alkyl(C6-C30)arylamino; a (C1-C30)alkylcarbonyl; a (C1-C30)alkoxycarbonyl; a (C6-C30)arylcarbonyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl, preferably are at least one selected from the group consisting of deuterium; a halogen; a (C1-C30)alkyl; a halo(C1-C30)alkyl; a (C6-C30)aryl; a (C1-C30)alkyl(C6-C30)aryl; a 5- to 30-membered heteroaryl; a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di-(C1-C30)alkylamino; a mono- or di-(C6-C30)arylamino; a (C1-C30)alkyl(C6-C30)arylamino; a hydroxyl; and a (C1-C30)alkoxy, more preferably are at least one selected from the group consisting of deuterium; a halogen; a (C1-C6)alkyl; a (C6-C20)aryl; a (C1-C6)alkyl(C6-C20)aryl; a 5- to 20-membered heteroaryl; a di(C6-C12)arylamino; a tri(C6-C12)arylsilyl; and a (C1-C6)alkyldi(C6-C12)arylsilyl.

In formula (1) above, L₁ and L₂ each independently represent a single bond, a substituted or unsubstituted 5- to 30-membered heteroarylene, or a substituted or unsubstituted (C6-C30)arylene, preferably each independently represent a single bond, a substituted or unsubstituted 5- to 15-membered heteroarylene, or a substituted or unsubstituted (C6-C20)arylene, more preferably each independently represent a single bond; a 5- to 15-membered heteroarylene unsubstituted or substituted with a (C1-C6)alkyl or a (C6-C15)aryl; or a (C6-C20)arylene unsubstituted or substituted with a (C1-C6)alkyl or a (C6-C15)aryl.

X₁ and X₂ each independently represent CR₇ or N, where R₇ preferably represents hydrogen, a substituted or unsubstituted (C6-C30)aryl, or a substituted or unsubstituted 5- to 15-membered heteroaryl, more preferably represents hydrogen; a 5- to 15-membered heteroaryl unsubstituted or substituted with a (C1-C6)alkyl or a (C6-C15)aryl; or a (C6-C20)aryl unsubstituted or substituted with a (C1-C6)alkyl or a (C6-C15)aryl.

R₁ to R₄ each independently represent each independently represent hydrogen, deuterium, a halogen, a cyano, a carboxyl, a nitro, a hydroxyl, a substituted or unsubstituted (C1-C30)alkyl, a substituted or unsubstituted (C2-C30)alkenyl, a substituted or unsubstituted (C2-C30)alkynyl, a substituted or unsubstituted (C1-C30)alkoxy, a substituted or unsubstituted (C3-C30)cycloalkyl, a substituted or unsubstituted (C3-C30)cycloalkenyl, a substituted or unsubstituted 3- to 7-membered heterocycloalkyl, a substituted or unsubstituted (C6-C30)aryl, a substituted or unsubstituted 5- to 30-membered heteroaryl, —NR₁₁R₁₂, —SiR₁₃R₁₄R₁₅, —SR₁₆, —OR₁₇, —COR₁₈ or —B(OR₁₉)(OR₂₀); or are linked to an adjacent substituent(s) to form a mono- or polycyclic, 3- to 30-membered alicyclic or aromatic ring whose carbon atom(s) may be replaced with at least one hetero atom selected from the group consisting of nitrogen, oxygen and sulfur, preferably each independently represent hydrogen, a halogen, a substituted or unsubstituted (C6-C30)aryl, a substituted or unsubstituted 5- to 30-membered heteroaryl, —NR₁₁R₁₂ or —SiR₁₃R₁₄R₁₅; or are linked to an adjacent substituent(s) to form a mono- or polycyclic, 3- to 30-membered alicyclic or aromatic ring, more preferably each independently represent hydrogen; a halogen; a (C6-C20)aryl unsubstituted or substituted with a (C1-C6)alkyl; a 5- to 15-membered heteroaryl unsubstituted or substituted with a (C1-C6)alkyl; —NR₁₁R₁₂; or —SiR₁₃R₁₄R₁₅, or are linked to an adjacent substituent(s) to form a mono- or polycyclic, 3- to 15-membered aromatic ring. Herein, R₁₁ and R₁₂ preferably each independently represent a substituted or unsubstituted (C6-C30)aryl, or a substituted or unsubstituted 5- to 30-membered heteroaryl, more preferably each independently represent an unsubstituted (C6-C20)aryl, or an unsubstituted 5- to 15-membered heteroaryl. R₁₃ to R₁₅ preferably each independently represent a substituted or unsubstituted (C1-C30)alkyl, or a substituted or unsubstituted (C6-C30)aryl, more preferably each independently represent an unsubstituted (C1-C10)alkyl, or an unsubstituted (C6-C15)aryl.

R₅ and R₆ each independently represent hydrogen, deuterium, a halogen, a substituted or unsubstituted (C1-C30)alkyl, a substituted or unsubstituted (C6-C30)aryl, a substituted or unsubstituted 5- to 30-membered heteroaryl, —NR₁₁R₁₂ or —SiR₁₃R₁₄R₁₅, preferably each independently represent hydrogen, a substituted or unsubstituted (C6-C30)aryl, a substituted or unsubstituted 5- to 30-membered heteroaryl, or —SiR₁₃R₁₄R₁₅, more preferably each independently represent hydrogen; a substituted or unsubstituted (C6-C20)aryl; a 5- to 15-membered heteroaryl unsubstituted or substituted with a (C1-C6)alkyl or a (C6-C12)aryl; or —SiR₁₃R₁₄R₁₅. Herein, the substituted (C6-C30)aryl is preferably substituted with deuterium, a halogen, a (C1-C6)alkyl, a (C6-C20)aryl, a (C1-C6)alkyl(C6-C20)aryl, a 5- to 15-membered heteroaryl, a di(C6-C15)arylamino, a tri(C6-C15)arylsilyl, or a (C1-C6)alkyldi(C6-C15)arylsilyl. R₁₃ to R₁₅ preferably each independently represent a substituted or unsubstituted (C1-C30)alkyl, or a substituted or unsubstituted (C6-C30)aryl, more preferably each independently represent an unsubstituted (C1-C10)alkyl, or an unsubstituted (C6-C15)aryl.

According to one embodiment of the present invention in formula (1) above, L₁ and L₂ each independently represent a single bond, a substituted or unsubstituted 5- to 15-membered heteroarylene, or a substituted or unsubstituted (C6-C20)arylene; X₁ and X₂ each independently represent CR₇ or N; where R₇ represents hydrogen, a substituted or unsubstituted (C6-C30)aryl, or a substituted or unsubstituted 5- to 15-membered heteroaryl; R₁ to R₄ each independently represent hydrogen, a halogen, a substituted or unsubstituted (C6-C30)aryl, a substituted or unsubstituted 5- to 30-membered heteroaryl, —NR₁₁R₁₂ or —SiR₁₃R₁₄R₁₅; or are linked to an adjacent substituent(s) to form a mono- or polycyclic, 3- to 30-membered alicyclic or aromatic ring; where R₁₁ and R₁₂ each independently represent a substituted or unsubstituted (C6-C30)aryl, or a substituted or unsubstituted 5- to 30-membered heteroaryl, and R₁₃ to R₁₅ each independently represent a substituted or unsubstituted (C1-C30)alkyl, or a substituted or unsubstituted (C6-C30)aryl; R₅ and R₆ each independently represent hydrogen, a substituted or unsubstituted (C6-C30)aryl, a substituted or unsubstituted 5- to 30-membered heteroaryl, or —SiR₁₃R₁₄R₁₅; where R₁₃ to R₁₅ each independently represent a substituted or unsubstituted (C1-C30)alkyl, or a substituted or unsubstituted (C6-C30)aryl.

According to another embodiment of the present invention in formula (1) above, L₁ and L₂ each independently represent a single bond; a 5- to 15-membered heteroarylene unsubstituted or substituted with a (C1-C6)alkyl or a (C6-C15)aryl; or a (C6-C20)arylene unsubstituted or substituted with a (C1-C6)alkyl or a (C6-C15)aryl; X₁ and X₂ each independently represent CR₇ or N; where R₇ represents hydrogen; a 5- to 15-membered heteroaryl unsubstituted or substituted with a (C1-C6)alkyl or a (C6-C15)aryl; or a (C6-C20)aryl unsubstituted or substituted with a (C1-C6)alkyl or a (C6-C15)aryl; R₁ to R₄ each independently represent hydrogen; a halogen; a (C6-C20)aryl unsubstituted or substituted with a (C1-C6)alkyl; a 5- to 15-membered heteroaryl unsubstituted or substituted with a (C1-C6)alkyl; —NR₁₁R₁₂; or —SiR₁₃R₁₄R₁₅, or are linked to an adjacent substituent(s) to form a mono- or polycyclic, 3- to 15-membered aromatic ring; where R₁₁ and R₁₂ each independently represent an unsubstituted (C6-C20)aryl, or an unsubstituted 5- to 15-membered heteroaryl, and R₁₃ to R₁₅ each independently represent an unsubstituted (C1-C10)alkyl, or an unsubstituted (C6-C15)aryl; R₅ and R₆ each independently represent hydrogen; a (C6-C20)aryl unsubstituted or substituted with deuterium, a halogen, a (C1-C6)alkyl, a (C6-C20)aryl, a tri(C6-C15)arylsilyl, a di(C6-C15)arylamino, a (C1-C6)alkyl(C6-C20)aryl, a (C1-C6)alkyldi(C6-C15)arylsilyl or a 5- to 15-membered heteroaryl; a 5- to 15-membered heteroaryl unsubstituted or substituted with a (C1-C6)alkyl or a (C6-C12)aryl; or —SiR₁₃R₁₄R₁₅, where R₁₃ to R₁₅ each independently represent an unsubstituted (C1-C10)alkyl, or an unsubstituted (C6-C15)aryl.

The representative organic electroluminescent compounds of the present invention include the following compounds, but are not limited thereto:

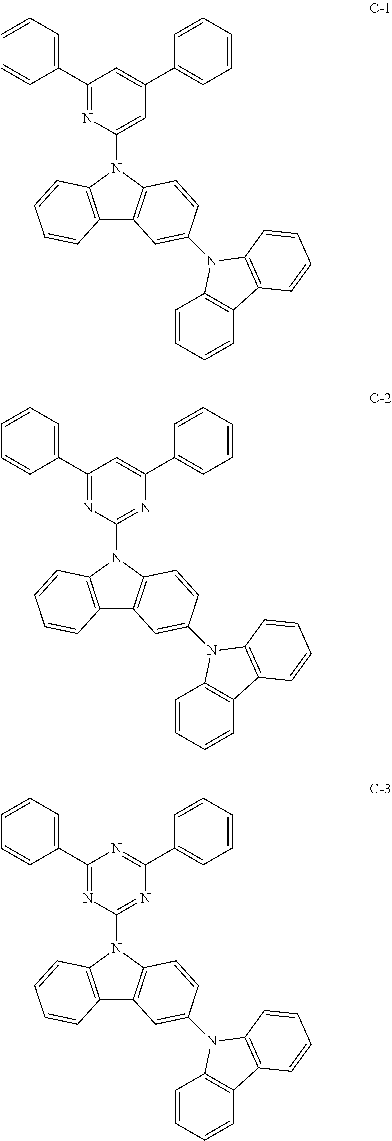 [SMILES: C=C/C=C(\C=C)c1cc(-c2ccccc2)cc(-n2c3ccccc3c3cc(-n4c5ccccc5c5ccccc54)ccc32)n1 ; c1ccc(-c2cc(-c3ccccc3)nc(-n3c4ccccc4c4cc(-n5c6ccccc6c6ccccc65)ccc43)n2)cc1 ; c1ccc(-c2nc(-c3ccccc3)nc(-n3c4ccccc4c4cc(-n5c6ccccc6c6ccccc65)ccc43)n2)cc1]

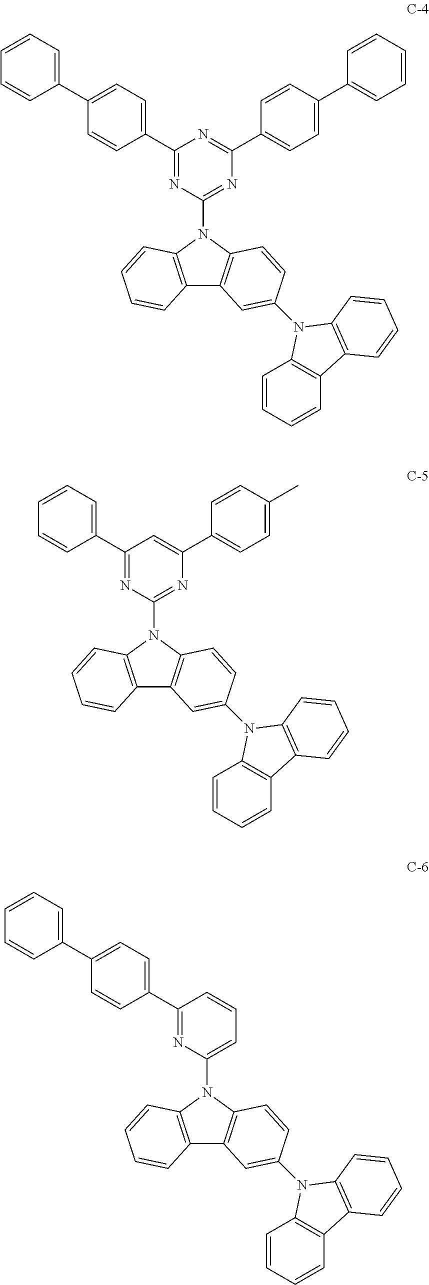 [SMILES: c1ccc(-c2ccc(-c3nc(-c4ccc(-c5ccccc5)cc4)nc(-n4c5ccccc5c5cc(-n6c7ccccc7c7ccccc76)ccc54)n3)cc2)cc1 ; Cc1ccc(-c2cc(-c3ccccc3)nc(-n3c4ccccc4c4cc(-n5c6ccccc6c6ccccc65)ccc43)n2)cc1 ; c1ccc(-c2ccc(-c3cccc(-n4c5ccccc5c5cc(-n6c7ccccc7c7ccccc76)ccc54)n3)cc2)cc1]

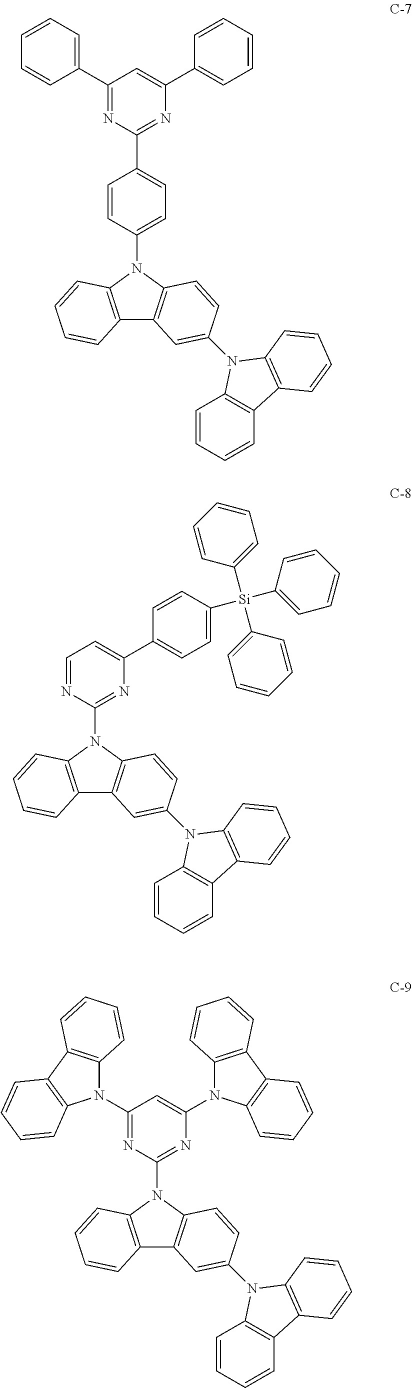

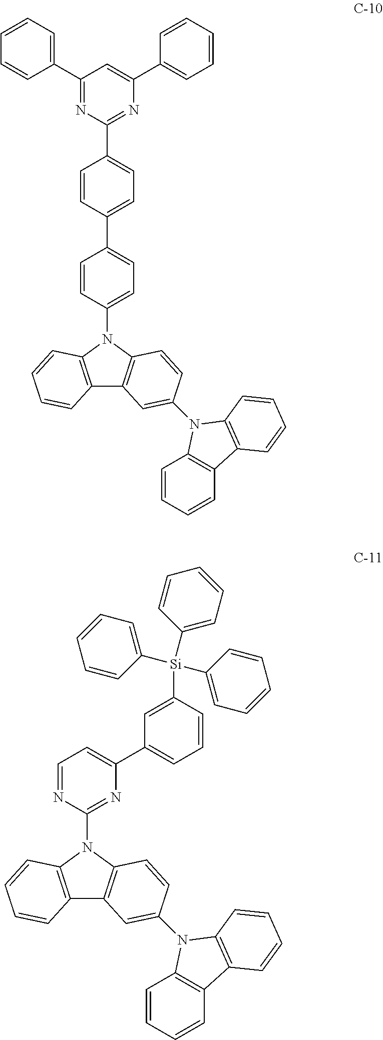 [SMILES: c1ccc(-c2cc(-c3ccccc3)nc(-c3ccc(-c4ccc(-n5c6ccccc6c6cc(-n7c8ccccc8c8ccccc87)ccc65)cc4)cc3)n2)cc1 ; c1ccc([Si](c2ccccc2)(c2ccccc2)c2cccc(-c3ccnc(-n4c5ccccc5c5cc(-n6c7ccccc7c7ccccc76)ccc54)n3)c2)cc1]

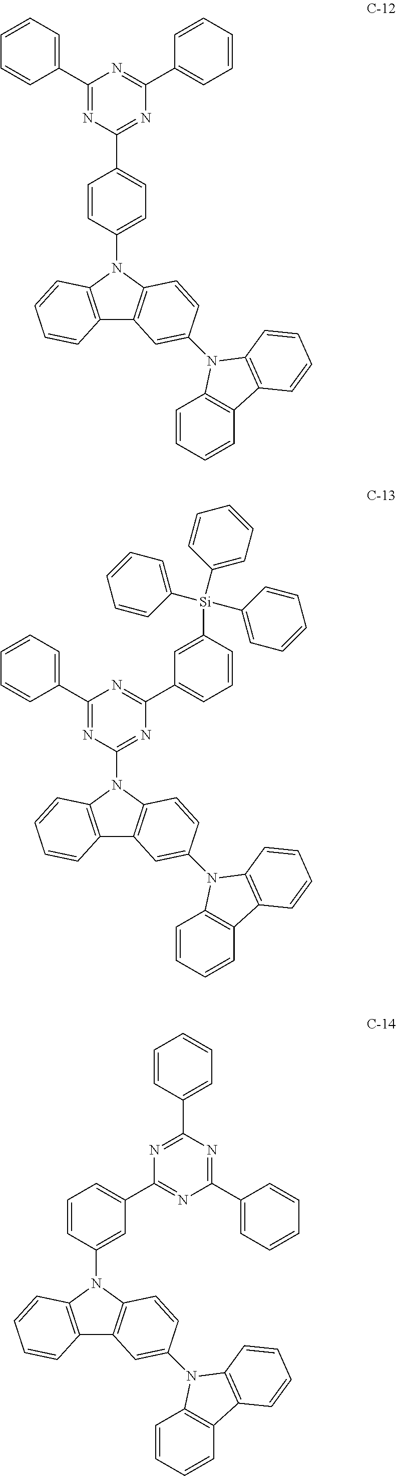 [SMILES: c1ccc(-c2nc(-c3ccccc3)nc(-c3ccc(-n4c5ccccc5c5cc(-n6c7ccccc7c7ccccc76)ccc54)cc3)n2)cc1 ; c1ccc(-c2nc(-c3cccc([Si](c4ccccc4)(c4ccccc4)c4ccccc4)c3)nc(-n3c4ccccc4c4cc(-n5c6ccccc6c6ccccc65)ccc43)n2)cc1 ; c1ccc(-c2nc(-c3ccccc3)nc(-c3cccc(-n4c5ccccc5c5cc(-n6c7ccccc7c7ccccc76)ccc54)c3)n2)cc1]

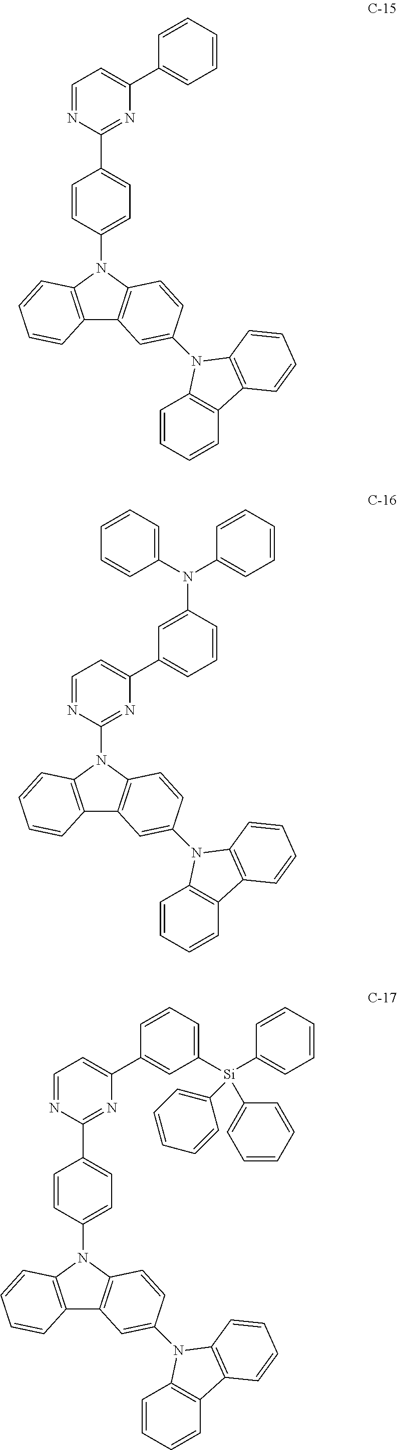 [SMILES: c1ccc(-c2ccnc(-c3ccc(-n4c5ccccc5c5cc(-n6c7ccccc7c7ccccc76)ccc54)cc3)n2)cc1 ; c1ccc(N(c2ccccc2)c2cccc(-c3ccnc(-n4c5ccccc5c5cc(-n6c7ccccc7c7ccccc76)ccc54)n3)c2)cc1 ; c1ccc([Si](c2ccccc2)(c2ccccc2)c2cccc(-c3ccnc(-c4ccc(-n5c6ccccc6c6cc(-n7c8ccccc8c8ccccc87)ccc65)cc4)n3)c2)cc1]

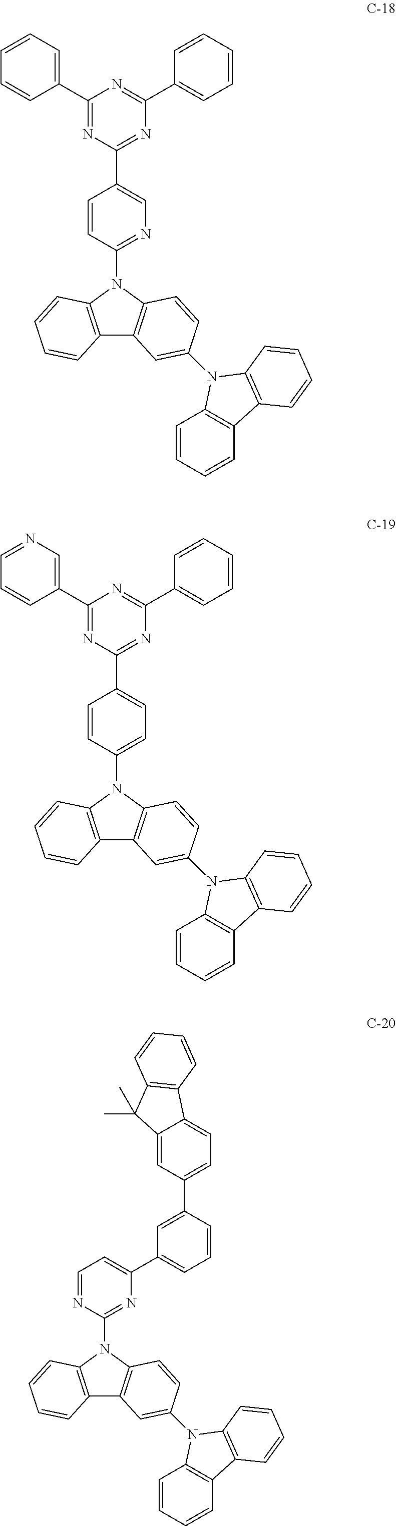 [SMILES: c1ccc(-c2nc(-c3ccccc3)nc(-c3ccc(-n4c5ccccc5c5cc(-n6c7ccccc7c7ccccc76)ccc54)nc3)n2)cc1 ; c1ccc(-c2nc(-c3ccc(-n4c5ccccc5c5cc(-n6c7ccccc7c7ccccc76)ccc54)cc3)nc(-c3cccnc3)n2)cc1 ; CC1(C)c2ccccc2-c2ccc(-c3cccc(-c4ccnc(-n5c6ccccc6c6cc(-n7c8ccccc8c8ccccc87)ccc65)n4)c3)cc21]

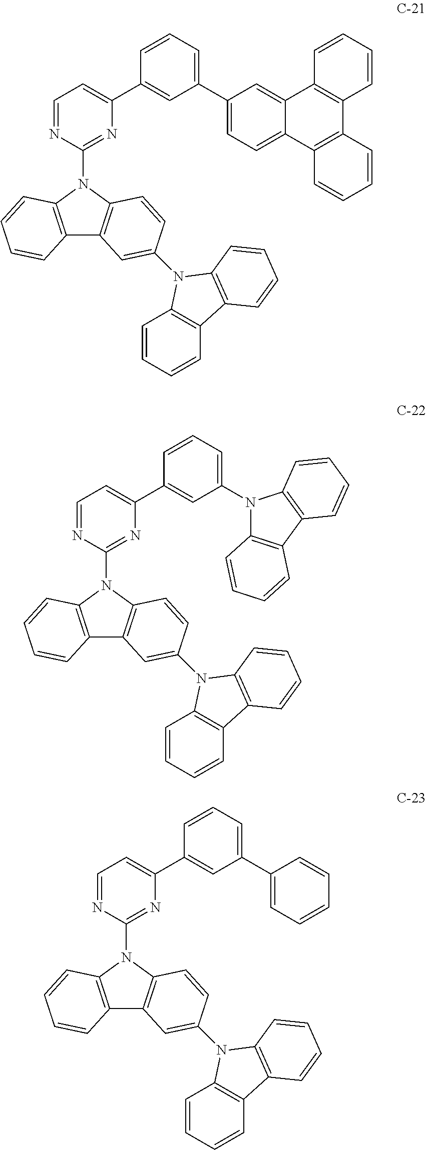

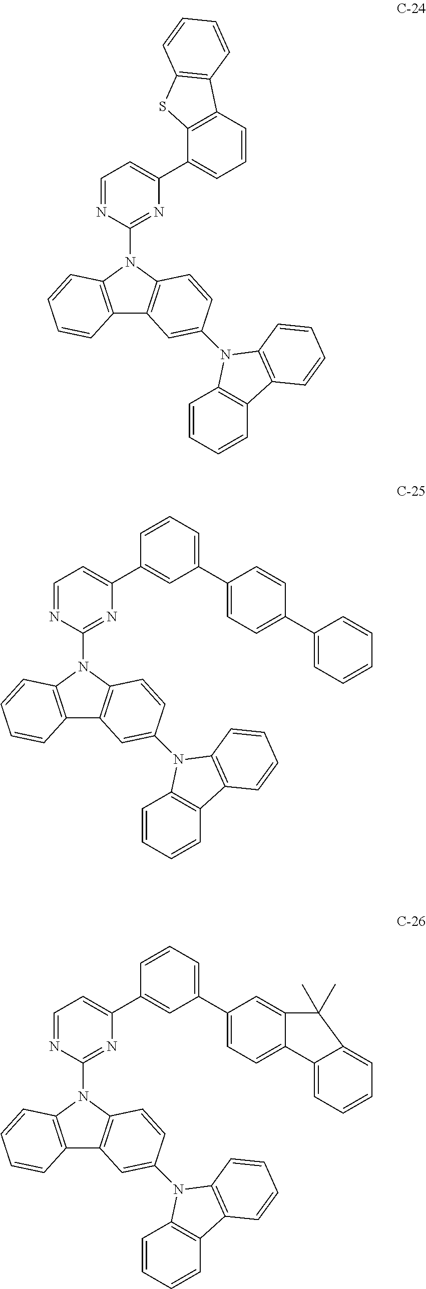 [SMILES: c1ccc2c(c1)sc1c(-c3ccnc(-n4c5ccccc5c5cc(-n6c7ccccc7c7ccccc76)ccc54)n3)cccc12 ; c1ccc(-c2ccc(-c3cccc(-c4ccnc(-n5c6ccccc6c6cc(-n7c8ccccc8c8ccccc87)ccc65)n4)c3)cc2)cc1 ; CC1(C)c2ccccc2-c2ccc(-c3cccc(-c4ccnc(-n5c6ccccc6c6cc(-n7c8ccccc8c8ccccc87)ccc65)n4)c3)cc21]

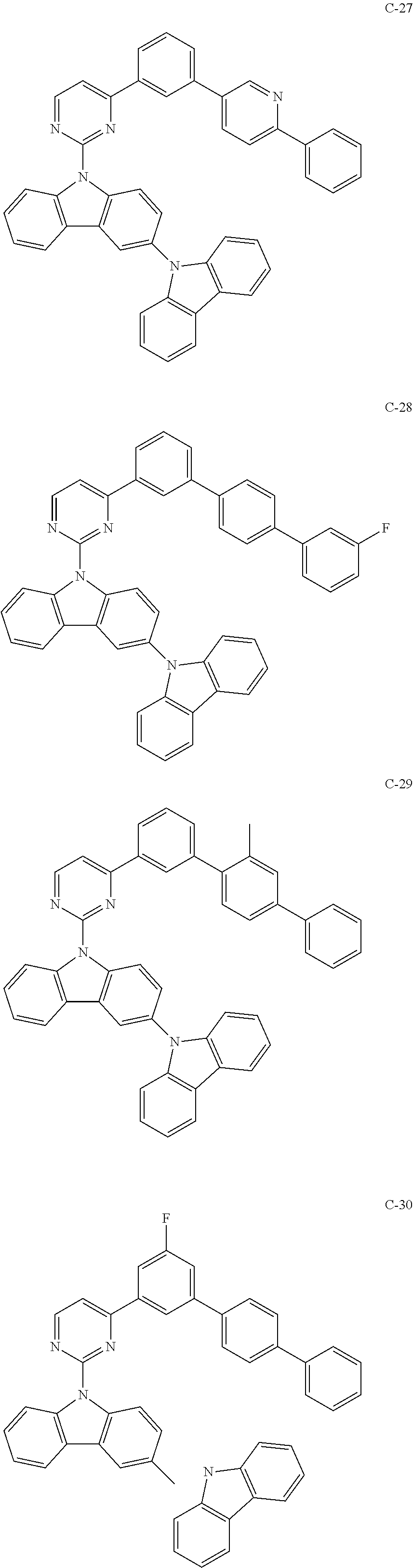

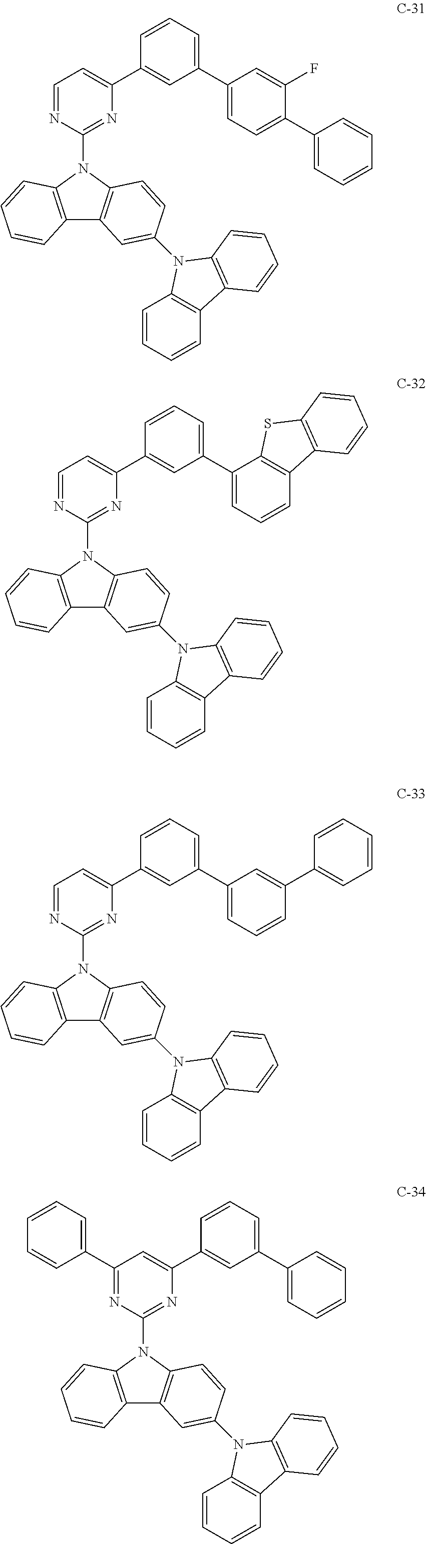

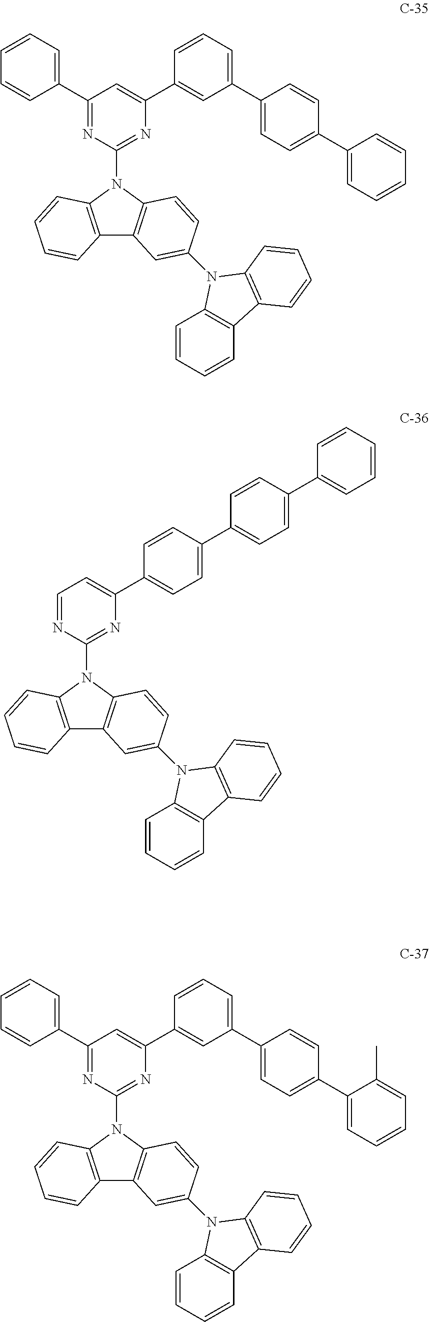 [SMILES: c1ccc(-c2ccc(-c3cccc(-c4cc(-c5ccccc5)nc(-n5c6ccccc6c6cc(-n7c8ccccc8c8ccccc87)ccc65)n4)c3)cc2)cc1 ; c1ccc(-c2ccc(-c3ccc(-c4ccnc(-n5c6ccccc6c6cc(-n7c8ccccc8c8ccccc87)ccc65)n4)cc3)cc2)cc1 ; Cc1ccccc1-c1ccc(-c2cccc(-c3cc(-c4ccccc4)nc(-n4c5ccccc5c5cc(-n6c7ccccc7c7ccccc76)ccc54)n3)c2)cc1]

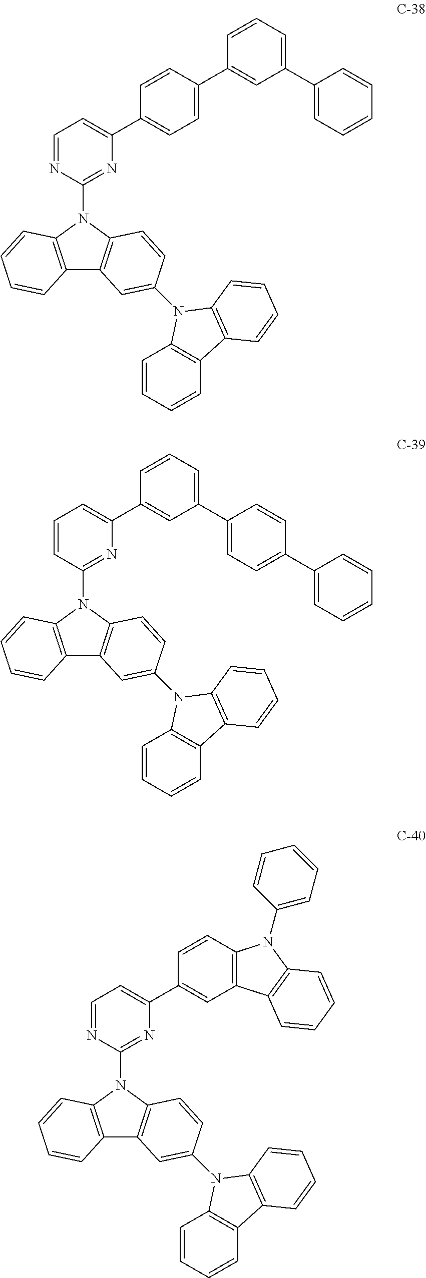

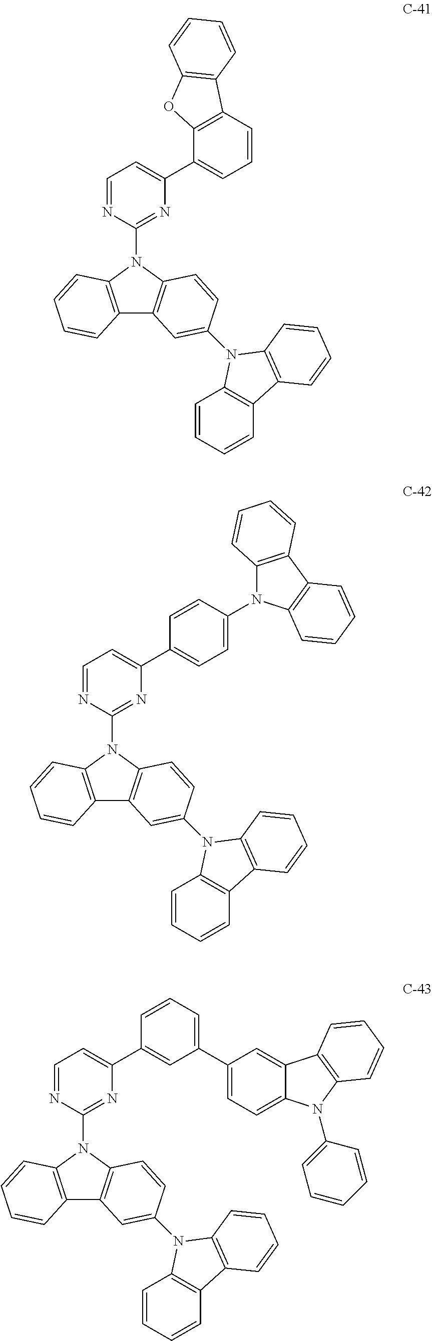 [SMILES: c1ccc2c(c1)oc1c(-c3ccnc(-n4c5ccccc5c5cc(-n6c7ccccc7c7ccccc76)ccc54)n3)cccc12 ; c1ccc2c(c1)c1ccccc1n2-c1ccc(-c2ccnc(-n3c4ccccc4c4cc(-n5c6ccccc6c6ccccc65)ccc43)n2)cc1 ; c1ccc(-n2c3ccccc3c3cc(-c4cccc(-c5ccnc(-n6c7ccccc7c7cc(-n8c9ccccc9c9ccccc98)ccc76)n5)c4)ccc32)cc1]

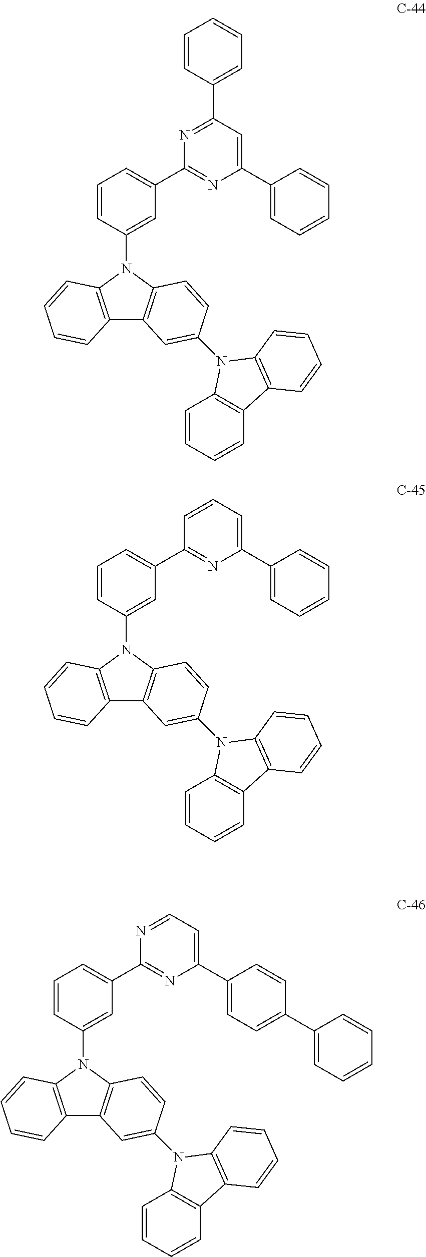

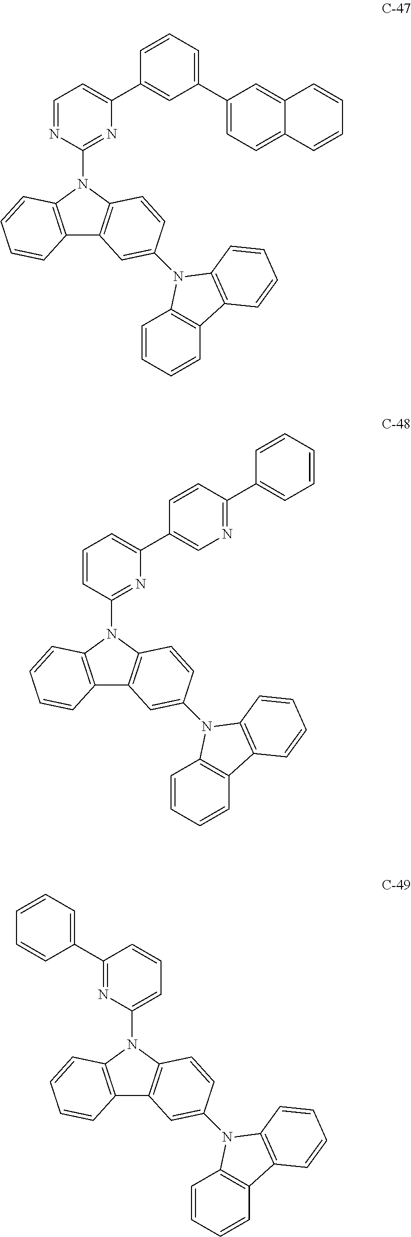 [SMILES: c1cc(-c2ccc3ccccc3c2)cc(-c2ccnc(-n3c4ccccc4c4cc(-n5c6ccccc6c6ccccc65)ccc43)n2)c1 ; c1ccc(-c2ccc(-c3cccc(-n4c5ccccc5c5cc(-n6c7ccccc7c7ccccc76)ccc54)n3)cn2)cc1 ; c1ccc(-c2cccc(-n3c4ccccc4c4cc(-n5c6ccccc6c6ccccc65)ccc43)n2)cc1]

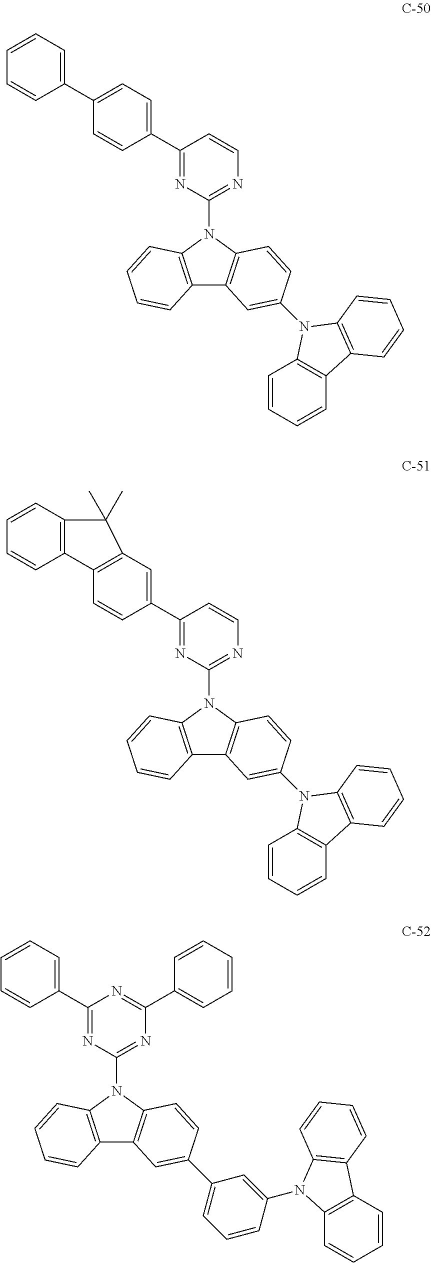 [SMILES: c1ccc(-c2ccc(-c3ccnc(-n4c5ccccc5c5cc(-n6c7ccccc7c7ccccc76)ccc54)n3)cc2)cc1 ; CC1(C)c2ccccc2-c2ccc(-c3ccnc(-n4c5ccccc5c5cc(-n6c7ccccc7c7ccccc76)ccc54)n3)cc21 ; c1ccc(-c2nc(-c3ccccc3)nc(-n3c4ccccc4c4cc(-c5cccc(-n6c7ccccc7c7ccccc76)c5)ccc43)n2)cc1]

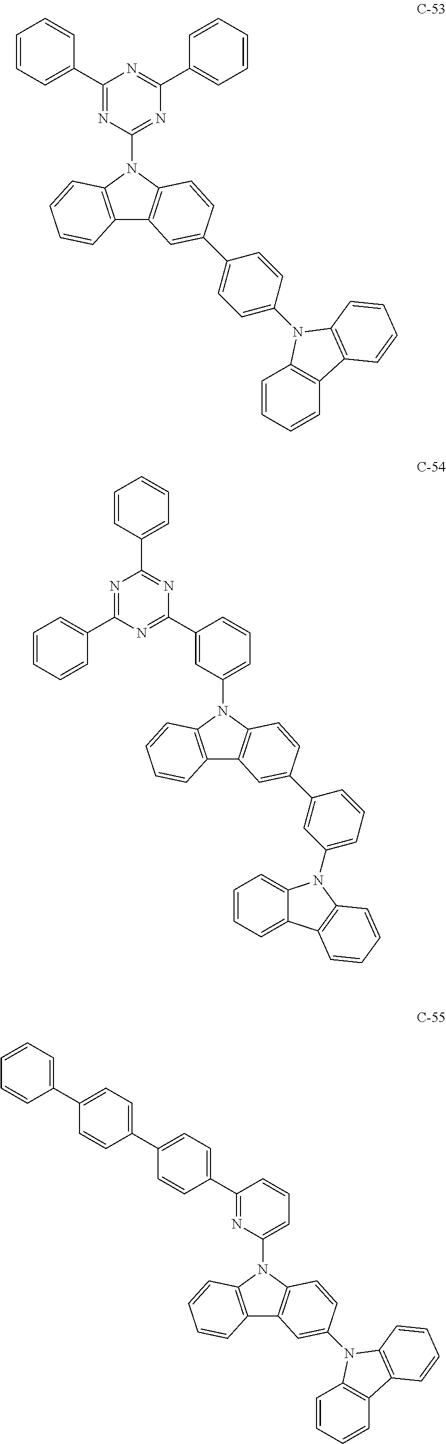 [SMILES: c1ccc(-c2nc(-c3ccccc3)nc(-n3c4ccccc4c4cc(-c5ccc(-n6c7ccccc7c7ccccc76)cc5)ccc43)n2)cc1 ; c1ccc(-c2nc(-c3ccccc3)nc(-c3cccc(-n4c5ccccc5c5cc(-c6cccc(-n7c8ccccc8c8ccccc87)c6)ccc54)c3)n2)cc1 ; c1ccc(-c2ccc(-c3ccc(-c4cccc(-n5c6ccccc6c6cc(-n7c8ccccc8c8ccccc87)ccc65)n4)cc3)cc2)cc1]

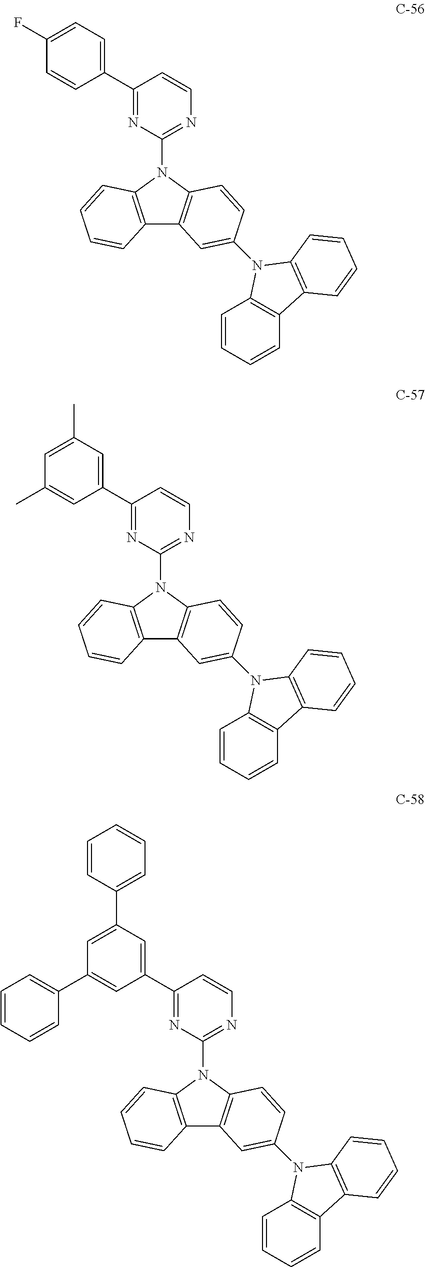 [SMILES: Fc1ccc(-c2ccnc(-n3c4ccccc4c4cc(-n5c6ccccc6c6ccccc65)ccc43)n2)cc1 ; Cc1cc(C)cc(-c2ccnc(-n3c4ccccc4c4cc(-n5c6ccccc6c6ccccc65)ccc43)n2)c1 ; c1ccc(-c2cc(-c3ccccc3)cc(-c3ccnc(-n4c5ccccc5c5cc(-n6c7ccccc7c7ccccc76)ccc54)n3)c2)cc1]

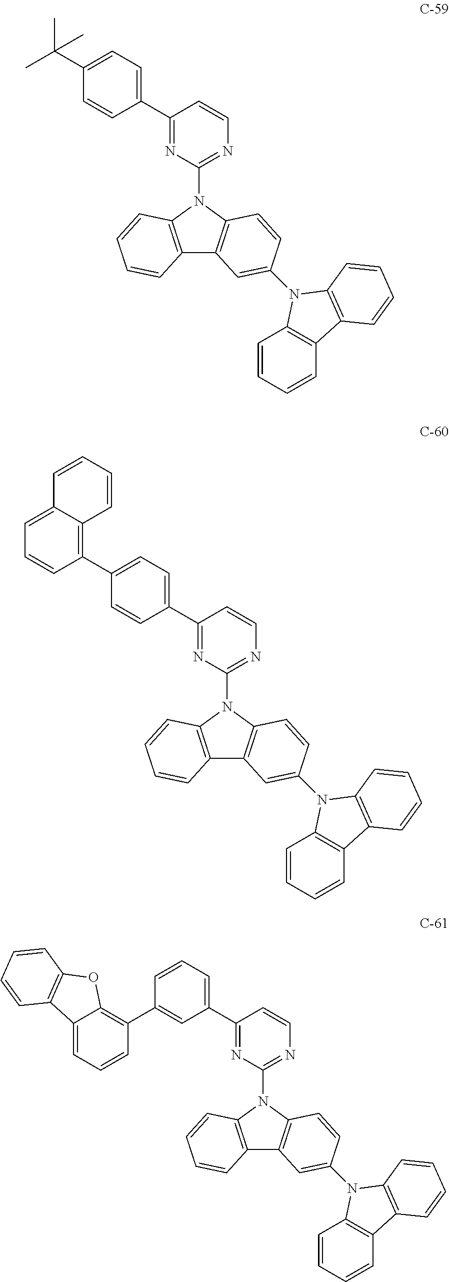 [SMILES: CC(C)(C)c1ccc(-c2ccnc(-n3c4ccccc4c4cc(-n5c6ccccc6c6ccccc65)ccc43)n2)cc1 ; c1ccc2c(-c3ccc(-c4ccnc(-n5c6ccccc6c6cc(-n7c8ccccc8c8ccccc87)ccc65)n4)cc3)cccc2c1 ; c1cc(-c2ccnc(-n3c4ccccc4c4cc(-n5c6ccccc6c6ccccc65)ccc43)n2)cc(-c2cccc3c2oc2ccccc23)c1]

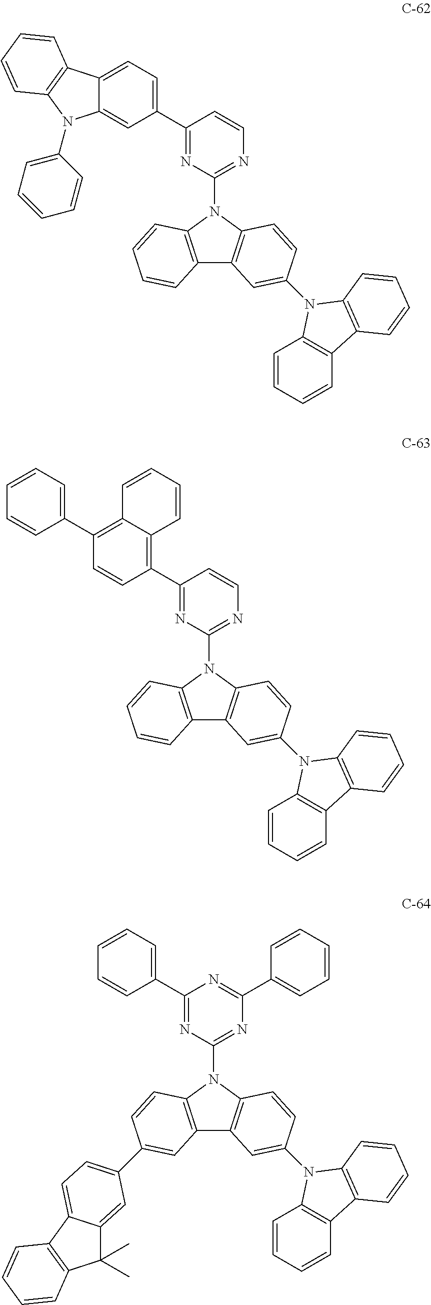 [SMILES: c1ccc(-n2c3ccccc3c3ccc(-c4ccnc(-n5c6ccccc6c6cc(-n7c8ccccc8c8ccccc87)ccc65)n4)cc32)cc1 ; c1ccc(-c2ccc(-c3ccnc(-n4c5ccccc5c5cc(-n6c7ccccc7c7ccccc76)ccc54)n3)c3ccccc23)cc1 ; CC1(C)c2ccccc2-c2ccc(-c3ccc4c(c3)c3cc(-n5c6ccccc6c6ccccc65)ccc3n4-c3nc(-c4ccccc4)nc(-c4ccccc4)n3)cc21]

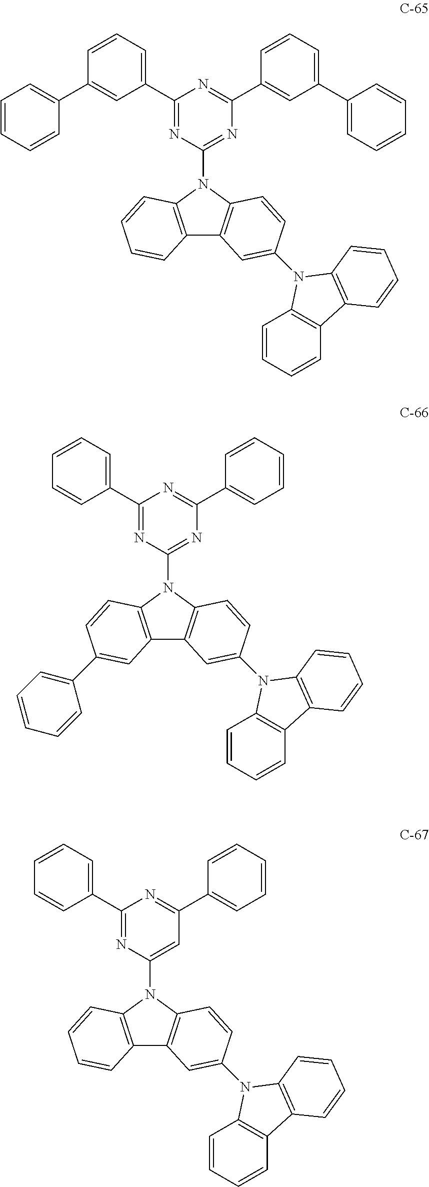 [SMILES: c1ccc(-c2cccc(-c3nc(-c4cccc(-c5ccccc5)c4)nc(-n4c5ccccc5c5cc(-n6c7ccccc7c7ccccc76)ccc54)n3)c2)cc1 ; c1ccc(-c2ccc3c(c2)c2cc(-n4c5ccccc5c5ccccc54)ccc2n3-c2nc(-c3ccccc3)nc(-c3ccccc3)n2)cc1 ; c1ccc(-c2cc(-n3c4ccccc4c4cc(-n5c6ccccc6c6ccccc65)ccc43)nc(-c3ccccc3)n2)cc1]

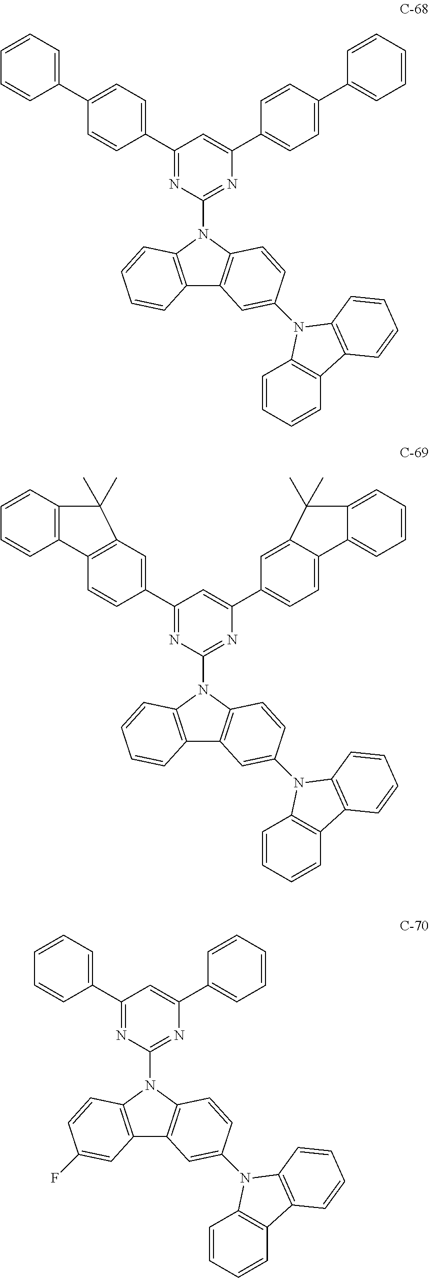 [SMILES: c1ccc(-c2ccc(-c3cc(-c4ccc(-c5ccccc5)cc4)nc(-n4c5ccccc5c5cc(-n6c7ccccc7c7ccccc76)ccc54)n3)cc2)cc1 ; CC1(C)c2ccccc2-c2ccc(-c3cc(-c4ccc5c(c4)C(C)(C)c4ccccc4-5)nc(-n4c5ccccc5c5cc(-n6c7ccccc7c7ccccc76)ccc54)n3)cc21 ; Fc1ccc2c(c1)c1cc(-n3c4ccccc4c4ccccc43)ccc1n2-c1nc(-c2ccccc2)cc(-c2ccccc2)n1]

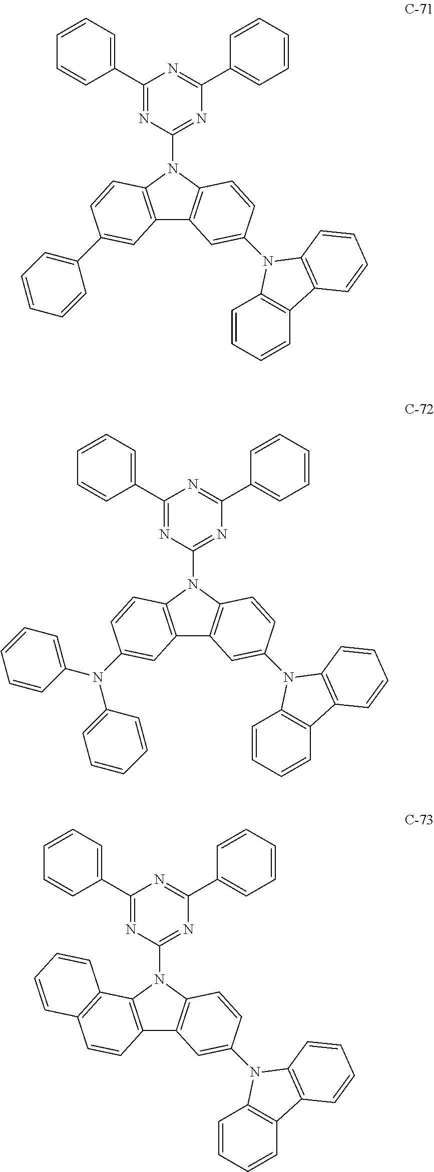 [SMILES: c1ccc(-c2ccc3c(c2)c2cc(-n4c5ccccc5c5ccccc54)ccc2n3-c2nc(-c3ccccc3)nc(-c3ccccc3)n2)cc1 ; c1ccc(-c2nc(-c3ccccc3)nc(-n3c4ccc(N(c5ccccc5)c5ccccc5)cc4c4cc(-n5c6ccccc6c6ccccc65)ccc43)n2)cc1 ; c1ccc(-c2nc(-c3ccccc3)nc(-n3c4ccc(-n5c6ccccc6c6ccccc65)cc4c4ccc5ccccc5c43)n2)cc1]

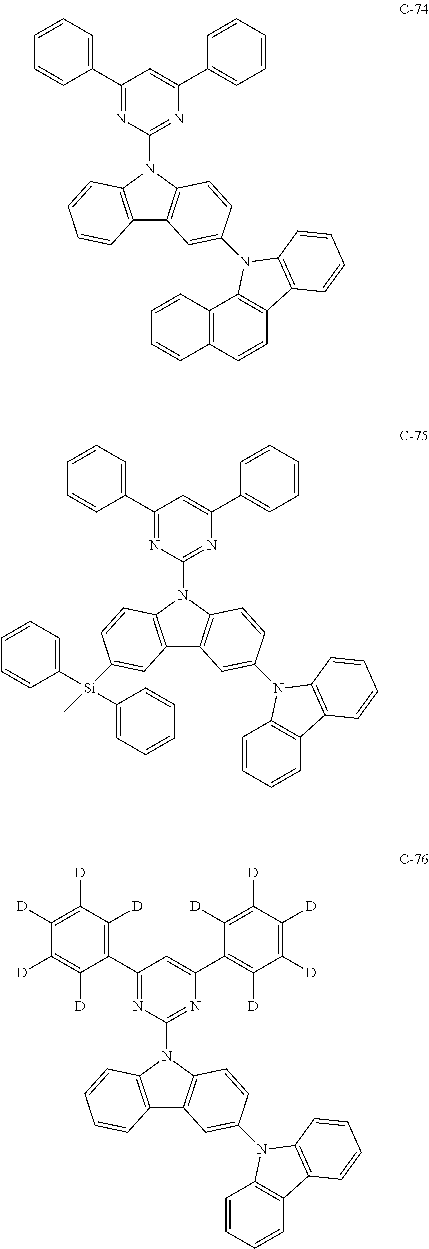 [SMILES: c1ccc(-c2cc(-c3ccccc3)nc(-n3c4ccccc4c4cc(-n5c6ccccc6c6ccc7ccccc7c65)ccc43)n2)cc1 ; C[Si](c1ccccc1)(c1ccccc1)c1ccc2c(c1)c1cc(-n3c4ccccc4c4ccccc43)ccc1n2-c1nc(-c2ccccc2)cc(-c2ccccc2)n1 ; [2H]c1c([2H])c([2H])c(-c2cc(-c3c([2H])c([2H])c([2H])c([2H])c3[2H])nc(-n3c4ccccc4c4cc(-n5c6ccccc6c6ccccc65)ccc43)n2)c([2H])c1[2H]]

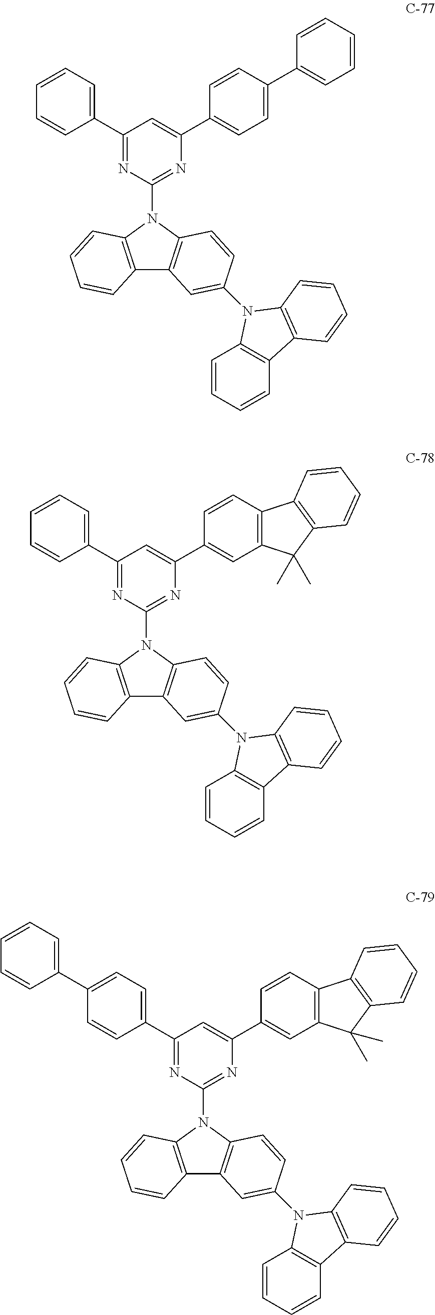 [SMILES: c1ccc(-c2ccc(-c3cc(-c4ccccc4)nc(-n4c5ccccc5c5cc(-n6c7ccccc7c7ccccc76)ccc54)n3)cc2)cc1 ; CC1(C)c2ccccc2-c2ccc(-c3cc(-c4ccccc4)nc(-n4c5ccccc5c5cc(-n6c7ccccc7c7ccccc76)ccc54)n3)cc21 ; CC1(C)c2ccccc2-c2ccc(-c3cc(-c4ccc(-c5ccccc5)cc4)nc(-n4c5ccccc5c5cc(-n6c7ccccc7c7ccccc76)ccc54)n3)cc21]

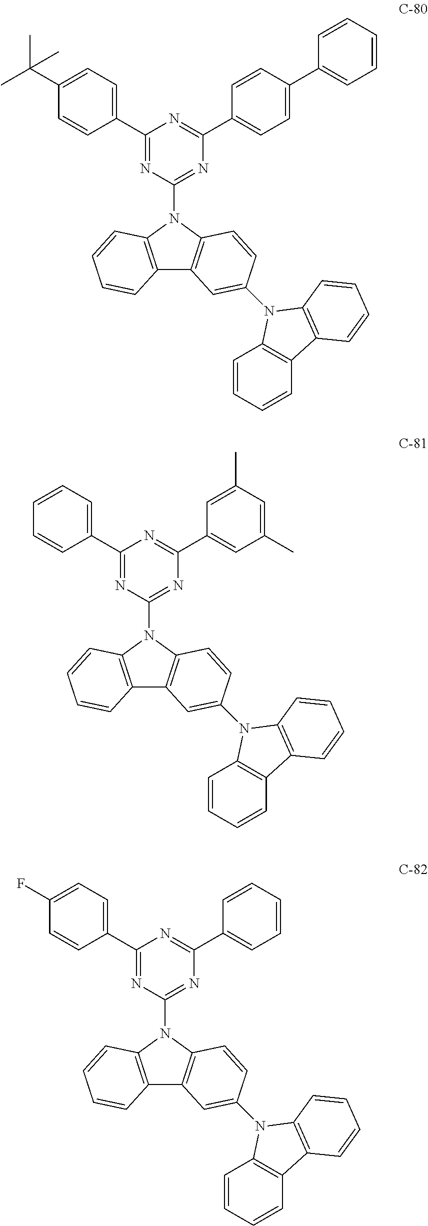 [SMILES: CC(C)(C)c1ccc(-c2nc(-c3ccc(-c4ccccc4)cc3)nc(-n3c4ccccc4c4cc(-n5c6ccccc6c6ccccc65)ccc43)n2)cc1 ; Cc1cc(C)cc(-c2nc(-c3ccccc3)nc(-n3c4ccccc4c4cc(-n5c6ccccc6c6ccccc65)ccc43)n2)c1 ; Fc1ccc(-c2nc(-c3ccccc3)nc(-n3c4ccccc4c4cc(-n5c6ccccc6c6ccccc65)ccc43)n2)cc1]

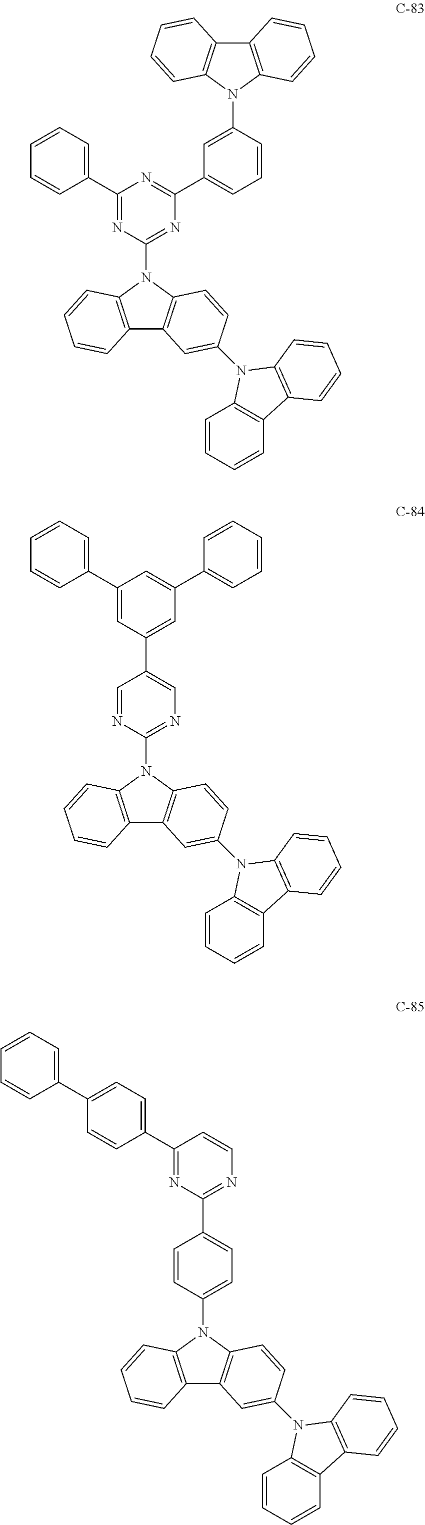 [SMILES: c1ccc(-c2nc(-c3cccc(-n4c5ccccc5c5ccccc54)c3)nc(-n3c4ccccc4c4cc(-n5c6ccccc6c6ccccc65)ccc43)n2)cc1 ; c1ccc(-c2cc(-c3ccccc3)cc(-c3cnc(-n4c5ccccc5c5cc(-n6c7ccccc7c7ccccc76)ccc54)nc3)c2)cc1 ; c1ccc(-c2ccc(-c3ccnc(-c4ccc(-n5c6ccccc6c6cc(-n7c8ccccc8c8ccccc87)ccc65)cc4)n3)cc2)cc1]

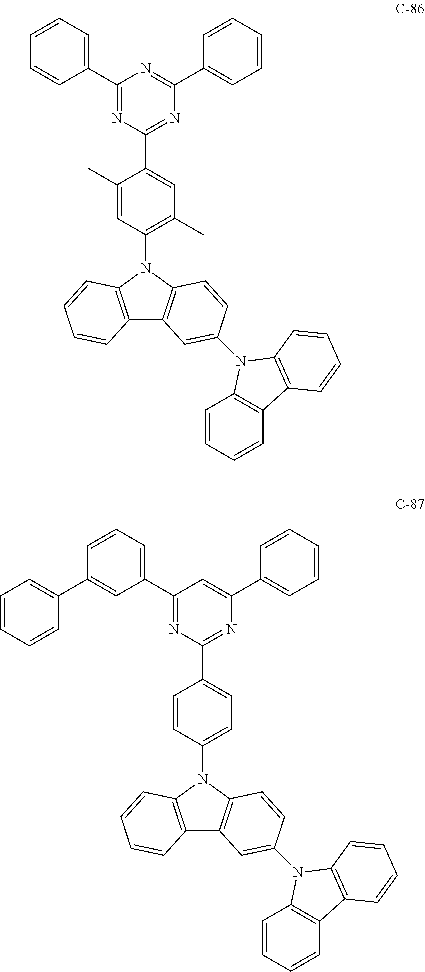 [SMILES: Cc1cc(-n2c3ccccc3c3cc(-n4c5ccccc5c5ccccc54)ccc32)c(C)cc1-c1nc(-c2ccccc2)nc(-c2ccccc2)n1 ; c1ccc(-c2cccc(-c3cc(-c4ccccc4)nc(-c4ccc(-n5c6ccccc6c6cc(-n7c8ccccc8c8ccccc87)ccc65)cc4)n3)c2)cc1]

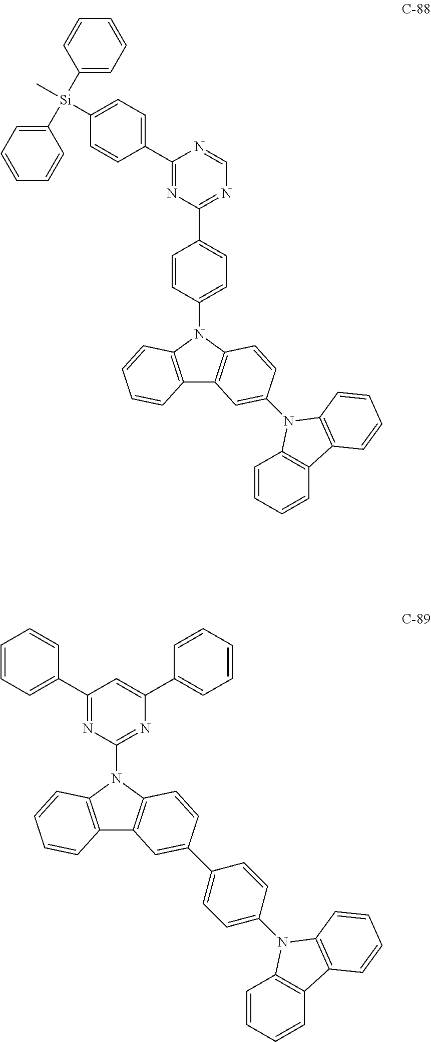 [SMILES: C[Si](c1ccccc1)(c1ccccc1)c1ccc(-c2ncnc(-c3ccc(-n4c5ccccc5c5cc(-n6c7ccccc7c7ccccc76)ccc54)cc3)n2)cc1 ; c1ccc(-c2cc(-c3ccccc3)nc(-n3c4ccccc4c4cc(-c5ccc(-n6c7ccccc7c7ccccc76)cc5)ccc43)n2)cc1]

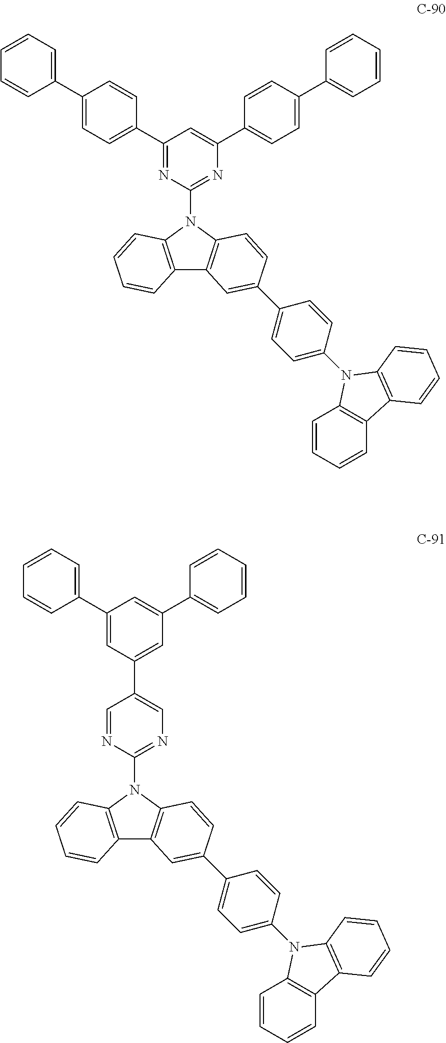 [SMILES: c1ccc(-c2ccc(-c3cc(-c4ccc(-c5ccccc5)cc4)nc(-n4c5ccccc5c5cc(-c6ccc(-n7c8ccccc8c8ccccc87)cc6)ccc54)n3)cc2)cc1 ; c1ccc(-c2cc(-c3ccccc3)cc(-c3cnc(-n4c5ccccc5c5cc(-c6ccc(-n7c8ccccc8c8ccccc87)cc6)ccc54)nc3)c2)cc1]

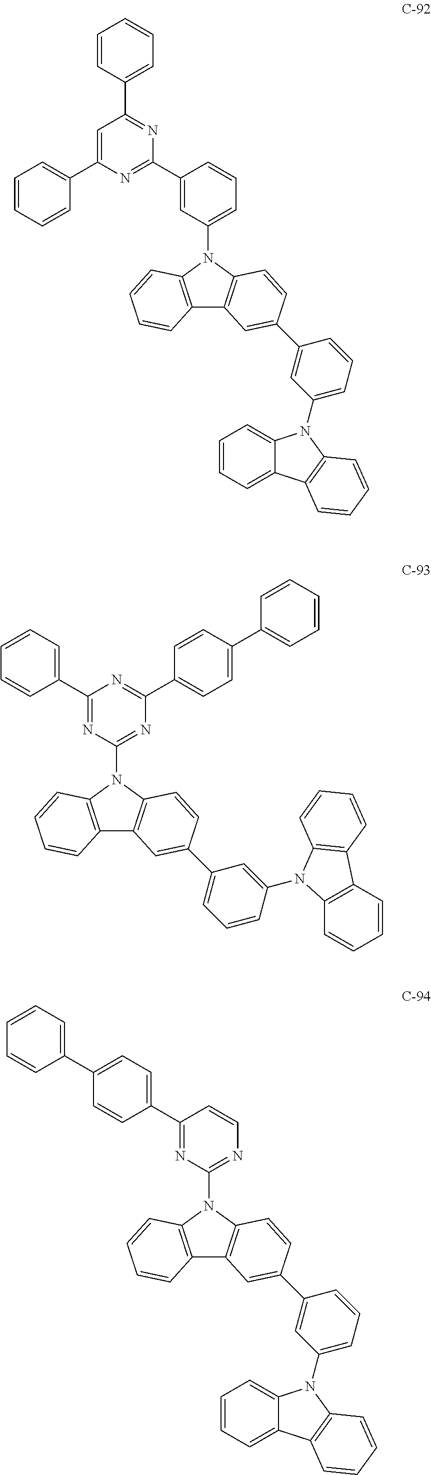 [SMILES: c1ccc(-c2cc(-c3ccccc3)nc(-c3cccc(-n4c5ccccc5c5cc(-c6cccc(-n7c8ccccc8c8ccccc87)c6)ccc54)c3)n2)cc1 ; c1ccc(-c2ccc(-c3nc(-c4ccccc4)nc(-n4c5ccccc5c5cc(-c6cccc(-n7c8ccccc8c8ccccc87)c6)ccc54)n3)cc2)cc1 ; c1ccc(-c2ccc(-c3ccnc(-n4c5ccccc5c5cc(-c6cccc(-n7c8ccccc8c8ccccc87)c6)ccc54)n3)cc2)cc1]

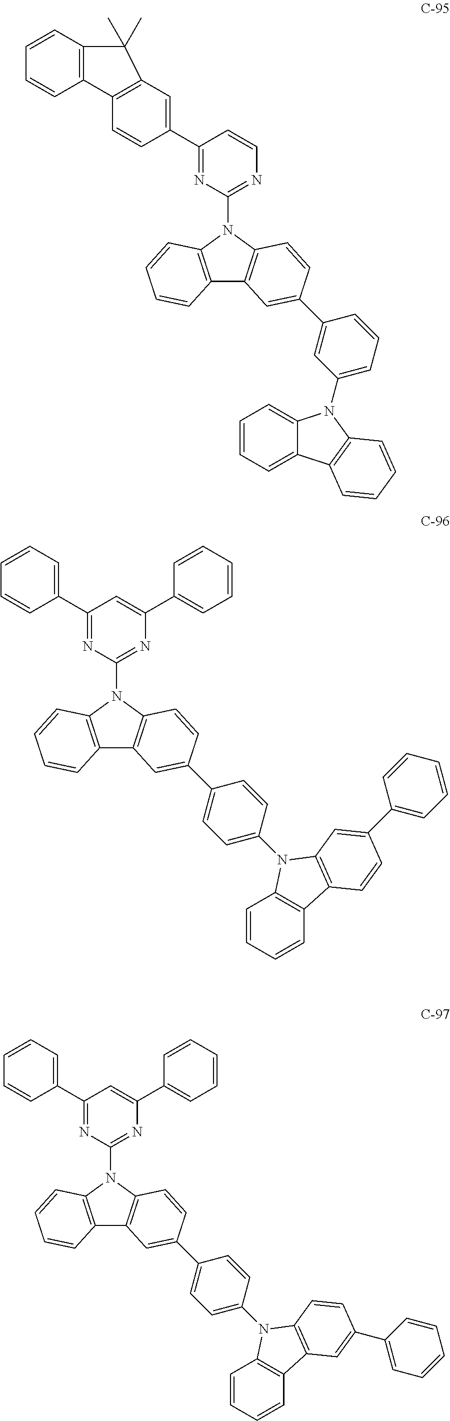 [SMILES: CC1(C)c2ccccc2-c2ccc(-c3ccnc(-n4c5ccccc5c5cc(-c6cccc(-n7c8ccccc8c8ccccc87)c6)ccc54)n3)cc21 ; c1ccc(-c2ccc3c4ccccc4n(-c4ccc(-c5ccc6c(c5)c5ccccc5n6-c5nc(-c6ccccc6)cc(-c6ccccc6)n5)cc4)c3c2)cc1 ; c1ccc(-c2ccc3c(c2)c2ccccc2n3-c2ccc(-c3ccc4c(c3)c3ccccc3n4-c3nc(-c4ccccc4)cc(-c4ccccc4)n3)cc2)cc1]

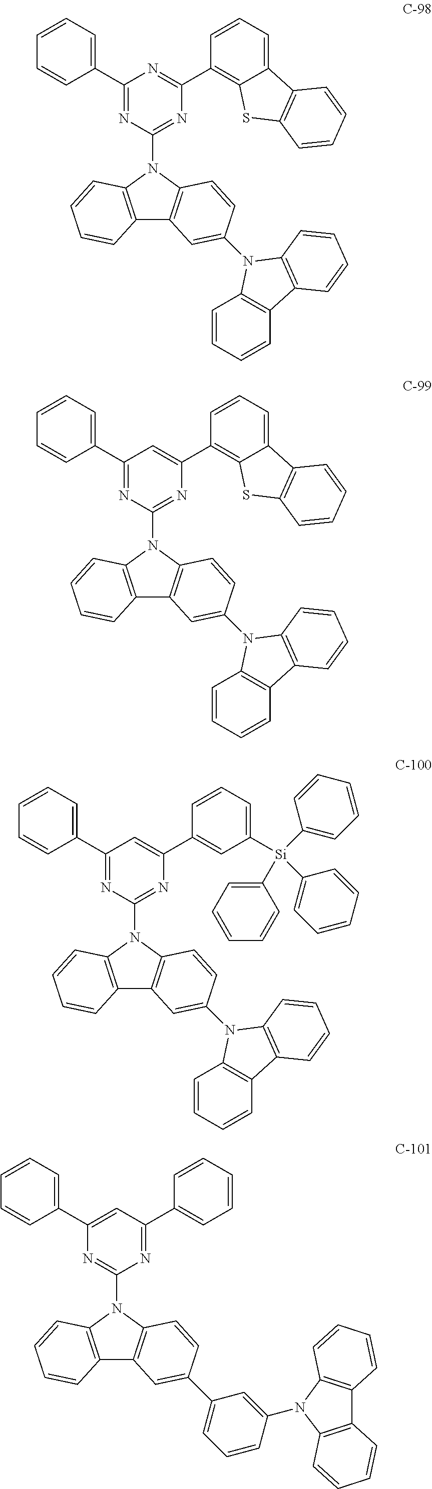

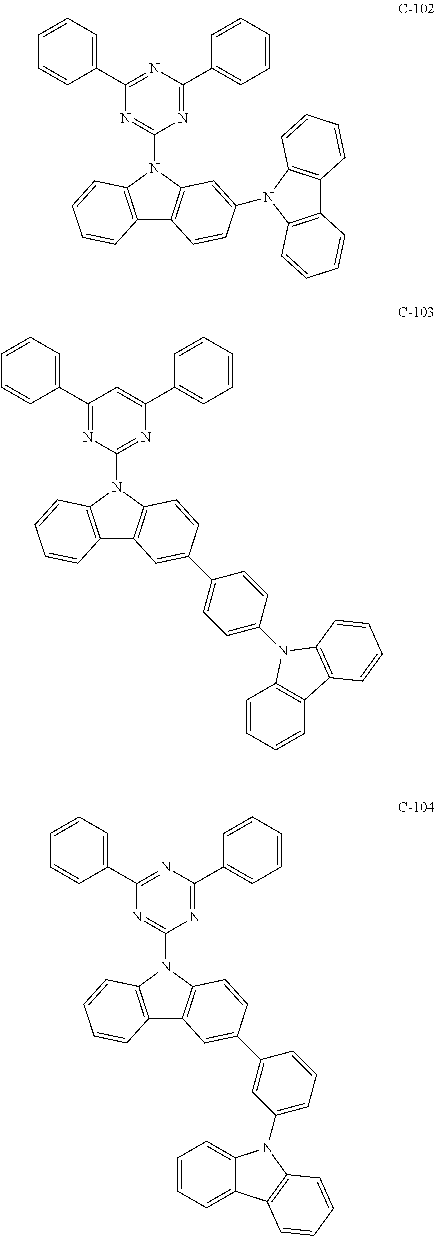 [SMILES: c1ccc(-c2nc(-c3ccccc3)nc(-n3c4ccccc4c4ccc(-n5c6ccccc6c6ccccc65)cc43)n2)cc1 ; c1ccc(-c2cc(-c3ccccc3)nc(-n3c4ccccc4c4cc(-c5ccc(-n6c7ccccc7c7ccccc76)cc5)ccc43)n2)cc1 ; c1ccc(-c2nc(-c3ccccc3)nc(-n3c4ccccc4c4cc(-c5cccc(-n6c7ccccc7c7ccccc76)c5)ccc43)n2)cc1]

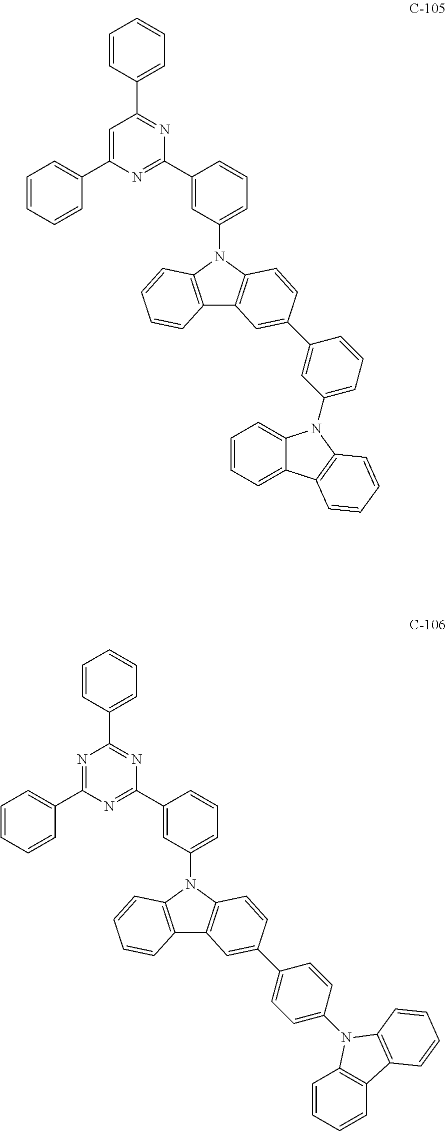 [SMILES: c1ccc(-c2cc(-c3ccccc3)nc(-c3cccc(-n4c5ccccc5c5cc(-c6cccc(-n7c8ccccc8c8ccccc87)c6)ccc54)c3)n2)cc1 ; c1ccc(-c2nc(-c3ccccc3)nc(-c3cccc(-n4c5ccccc5c5cc(-c6ccc(-n7c8ccccc8c8ccccc87)cc6)ccc54)c3)n2)cc1]

The organic electroluminescent compounds of the present invention can be prepared by a synthetic method known to a person skilled in the art such as a Suzuki reaction or an Ulman reaction. For example, they can be prepared according to the following reaction scheme 1.

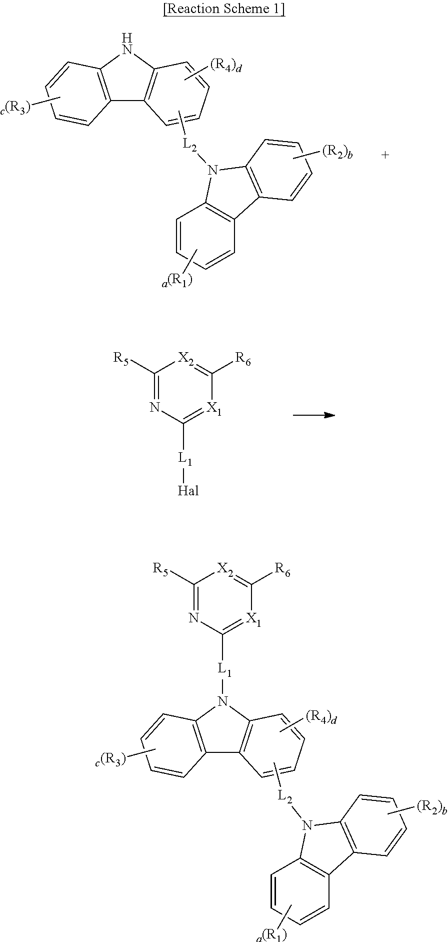

wherein L₁, L₂, R₁ to R₆, X₁, X₂, a, b, c and d are as defined in formula (1) above, and Hal represents a halogen.

In another embodiment of the present invention provides an organic electroluminescent material comprising the organic electroluminescent compound of formula (1), and an organic electroluminescent device comprising the material.

The above material can be comprised of the organic electroluminescent compound according to the present invention alone, or can further include conventional materials generally used in organic electroluminescent materials.

Said organic electroluminescent device comprises a first electrode, a second electrode, and at least one organic layer between said first and second electrodes. Said organic layer may comprise at least one organic electroluminescent compound of formula 1 according to the present invention.

One of the first and second electrodes is an anode, and the other is a cathode. The organic layer comprises a light-emitting layer, and at least one layer selected from the group consisting of a hole injection layer, a hole transport layer, an electron transport layer, an electron injection layer, an interlayer, and a hole blocking layer.

The organic electroluminescent compound according to the present invention can be comprised in the light-emitting layer. Where used in the light-emitting layer, the compound can be comprised as a host material. Preferably, the light-emitting layer can further comprise at least one dopant.

If needed, a compound other than the organic electroluminescent compound according to the present invention can be comprised additionally as a second host material.

The second host material can be from any of the known phosphorescent hosts. Specifically, the phosphorescent host selected from the group consisting of the compounds of formulas 5 to 9 below is preferable in view of luminous efficiency.

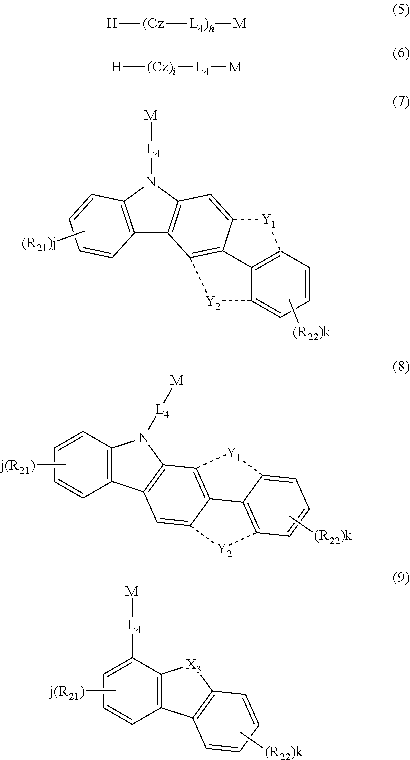

wherein Cz represents the following structure;

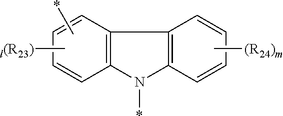

X₃ represents —O— or —S—;

R₂₁ to R₂₄ each independently represent hydrogen, deuterium, a halogen, a substituted or unsubstituted (C1-C30)alkyl group, a substituted of unsubstituted (C6-C30)aryl group, a substituted or unsubstituted 5- to 30-membered heteroaryl group, or R₂₅R₂₆R₂₇Si—;

R₂₅ to R₂₇ each independently represent a substituted or unsubstituted (C1-C30)alkyl group, or a substituted or unsubstituted (C6-C30)aryl group;

L₄ represents a single bond, a substituted or unsubstituted (C6-C30)arylene group, or a substituted or unsubstituted 5- to 30-membered heteroarylene group;

M represents a substituted or unsubstituted (C6-C30)aryl group, or a substituted or unsubstituted 5- to 30-membered heteroaryl group;

Y₁ and Y₂ each independently represent —O—, —S—, —N(R₃₁)— or —C(R₃₂)(R₃₃)—, provided that Y₁ and Y₂ do not simultaneously exist;

R₃₁ to R₃₃ each independently represent a substituted or unsubstituted (C1-C30)alkyl group, a substituted or unsubstituted (C6-C30)aryl group, or a substituted or unsubstituted 5- to 30-membered heteroaryl group, and R₃₂ and R₃₃ may be the same or different;

h and i each independently represent an integer of 1 to 3;

j, k, l and m each independently represent an integer of 0 to 4; and

where h, i, j, k, l or m is an integer of 2 or more, each of (Cz-L₄), each of (Cz), each of R₂₁, each of R₂₂, each of R₂₃ or each of R₂₄ may be the same or different.

Specifically, preferable examples of the second host material are as follows:

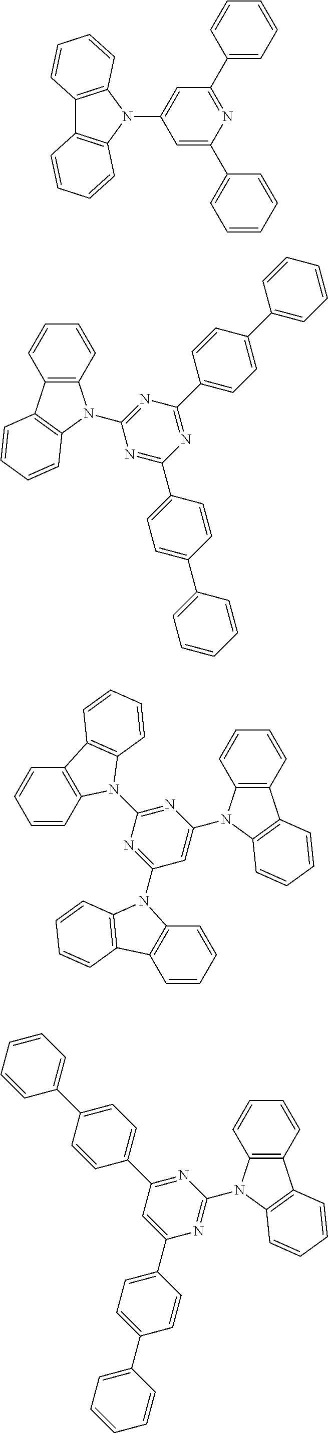

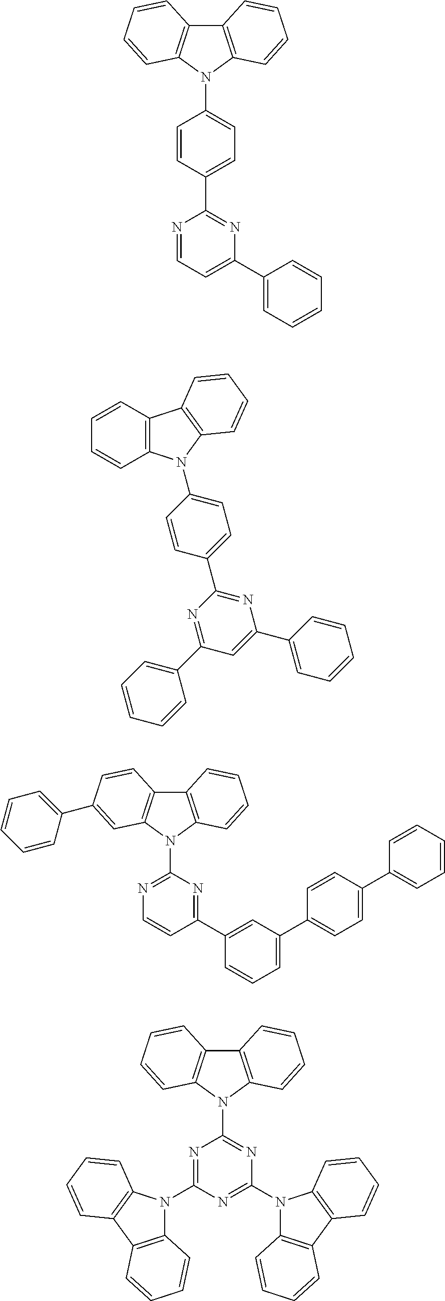

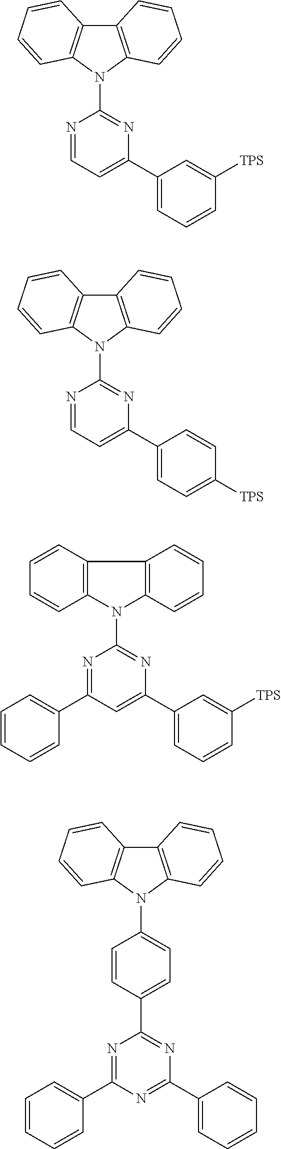

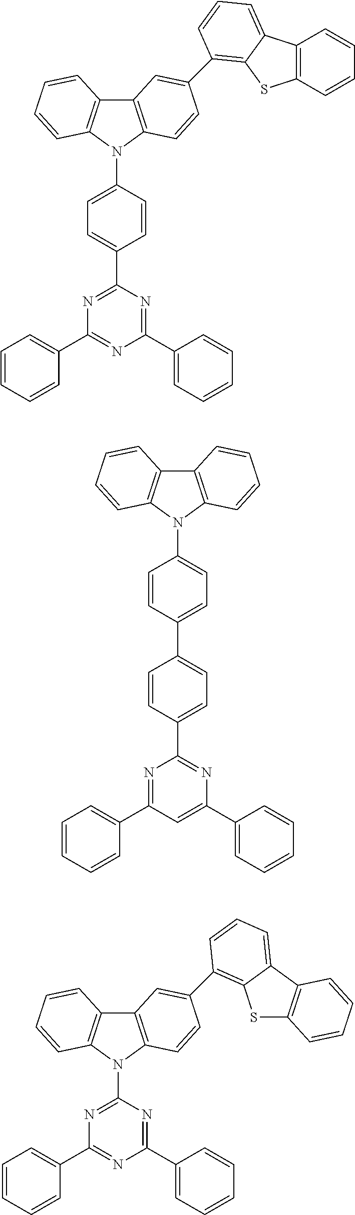 [SMILES: c1ccc(-c2nc(-c3ccccc3)nc(-c3ccc(-n4c5ccccc5c5cc(-c6cccc7c6sc6ccccc67)ccc54)cc3)n2)cc1 ; c1ccc(-c2cc(-c3ccccc3)nc(-c3ccc(-c4ccc(-n5c6ccccc6c6ccccc65)cc4)cc3)n2)cc1 ; c1ccc(-c2nc(-c3ccccc3)nc(-n3c4ccccc4c4cc(-c5cccc6c5sc5ccccc56)ccc43)n2)cc1]

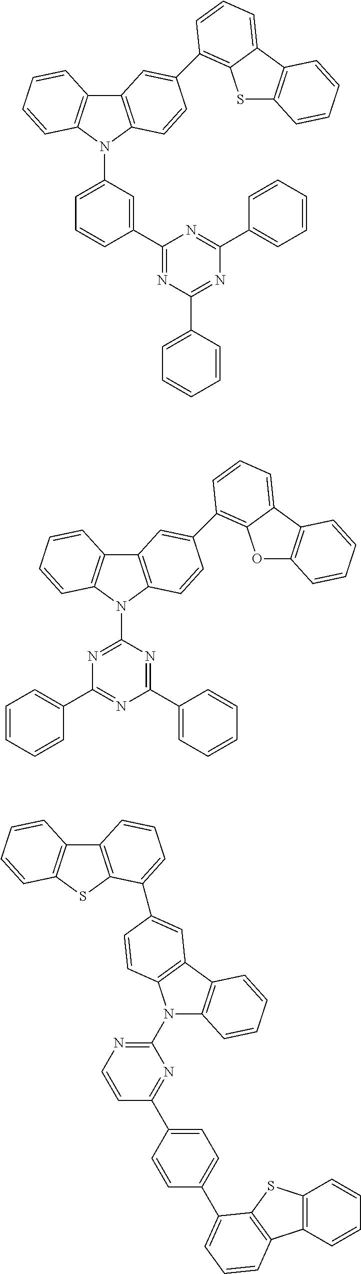 [SMILES: c1ccc(-c2nc(-c3ccccc3)nc(-c3cccc(-n4c5ccccc5c5cc(-c6cccc7c6sc6ccccc67)ccc54)c3)n2)cc1 ; c1ccc(-c2nc(-c3ccccc3)nc(-n3c4ccccc4c4cc(-c5cccc6c5oc5ccccc56)ccc43)n2)cc1 ; c1ccc2c(c1)sc1c(-c3ccc(-c4ccnc(-n5c6ccccc6c6cc(-c7cccc8c7sc7ccccc78)ccc65)n4)cc3)cccc12]

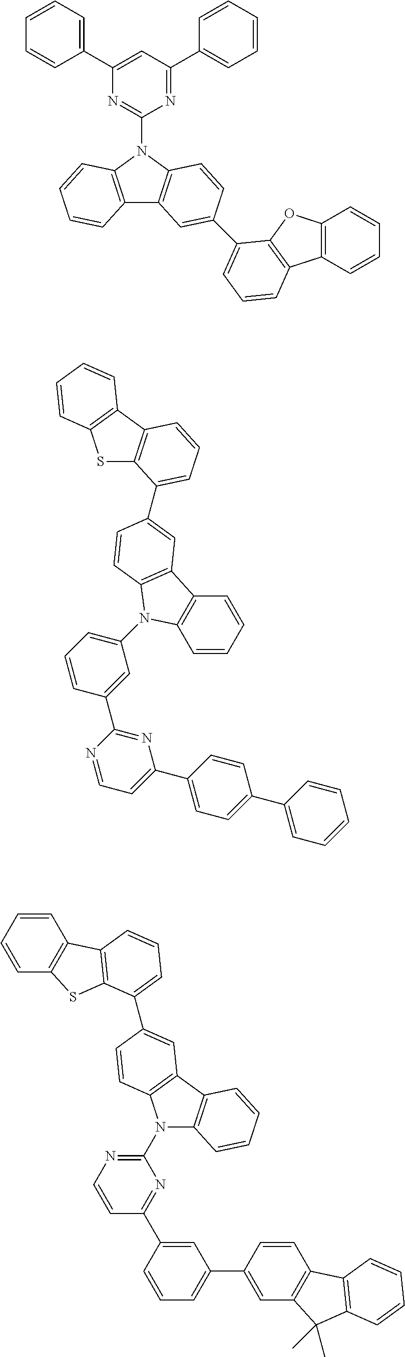 [SMILES: c1ccc(-c2cc(-c3ccccc3)nc(-n3c4ccccc4c4cc(-c5cccc6c5oc5ccccc56)ccc43)n2)cc1 ; c1ccc(-c2ccc(-c3ccnc(-c4cccc(-n5c6ccccc6c6cc(-c7cccc8c7sc7ccccc78)ccc65)c4)n3)cc2)cc1 ; CC1(C)c2ccccc2-c2ccc(-c3cccc(-c4ccnc(-n5c6ccccc6c6cc(-c7cccc8c7sc7ccccc78)ccc65)n4)c3)cc21]

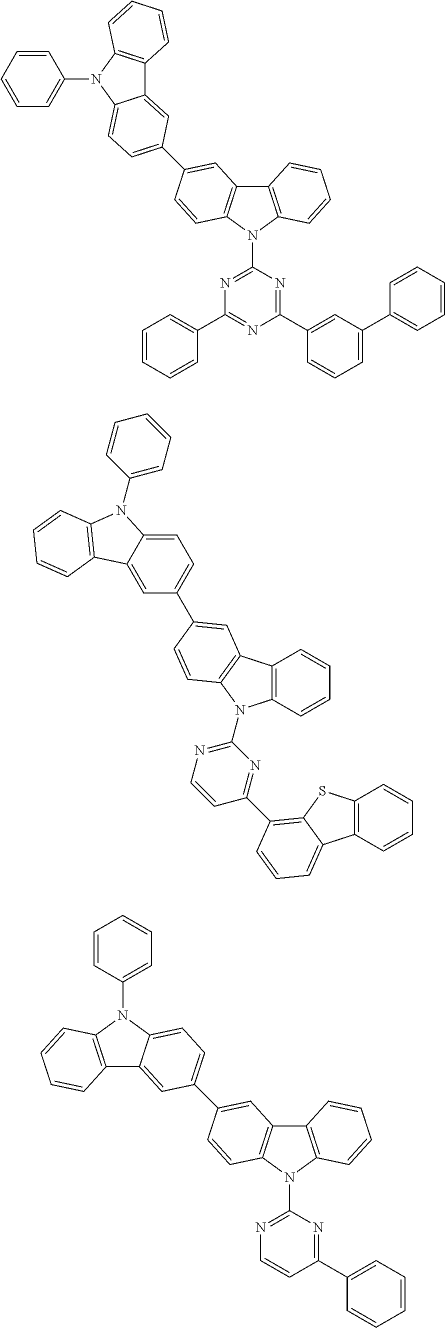

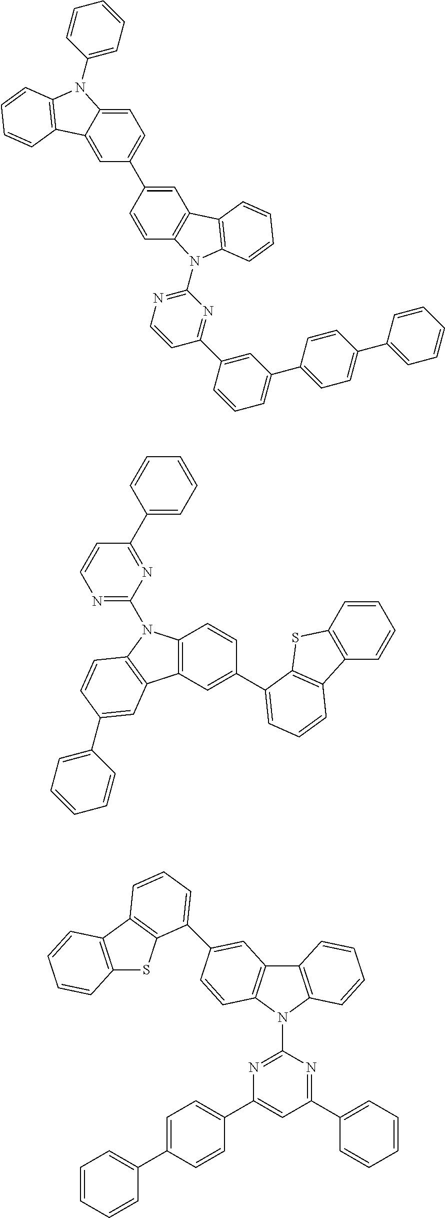 [SMILES: c1ccc(-c2ccc(-c3cccc(-c4ccnc(-n5c6ccccc6c6cc(-c7ccc8c(c7)c7ccccc7n8-c7ccccc7)ccc65)n4)c3)cc2)cc1 ; c1ccc(-c2ccc3c(c2)c2cc(-c4cccc5c4sc4ccccc45)ccc2n3-c2nccc(-c3ccccc3)n2)cc1 ; c1ccc(-c2ccc(-c3cc(-c4ccccc4)nc(-n4c5ccccc5c5cc(-c6cccc7c6sc6ccccc67)ccc54)n3)cc2)cc1]

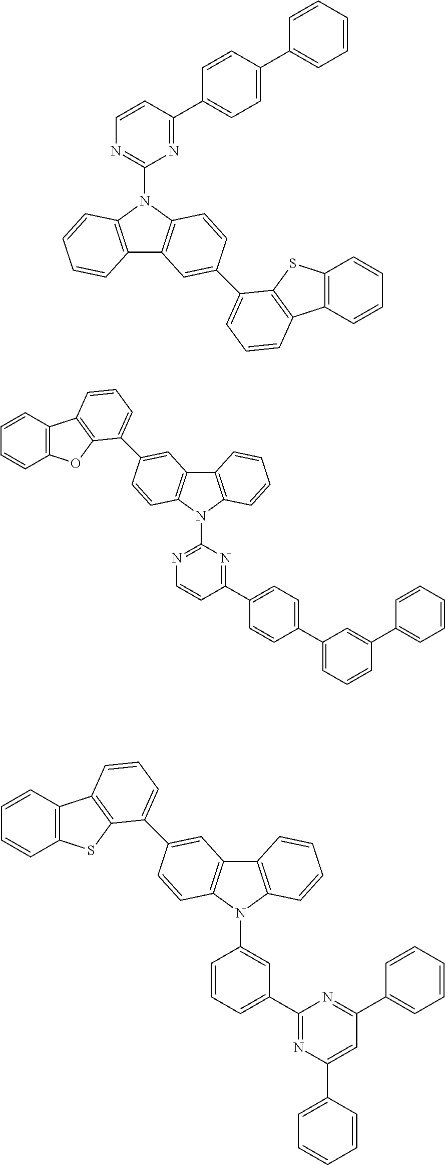 [SMILES: c1ccc(-c2ccc(-c3ccnc(-n4c5ccccc5c5cc(-c6cccc7c6sc6ccccc67)ccc54)n3)cc2)cc1 ; c1ccc(-c2cccc(-c3ccc(-c4ccnc(-n5c6ccccc6c6cc(-c7cccc8c7oc7ccccc78)ccc65)n4)cc3)c2)cc1 ; c1ccc(-c2cc(-c3ccccc3)nc(-c3cccc(-n4c5ccccc5c5cc(-c6cccc7c6sc6ccccc67)ccc54)c3)n2)cc1]

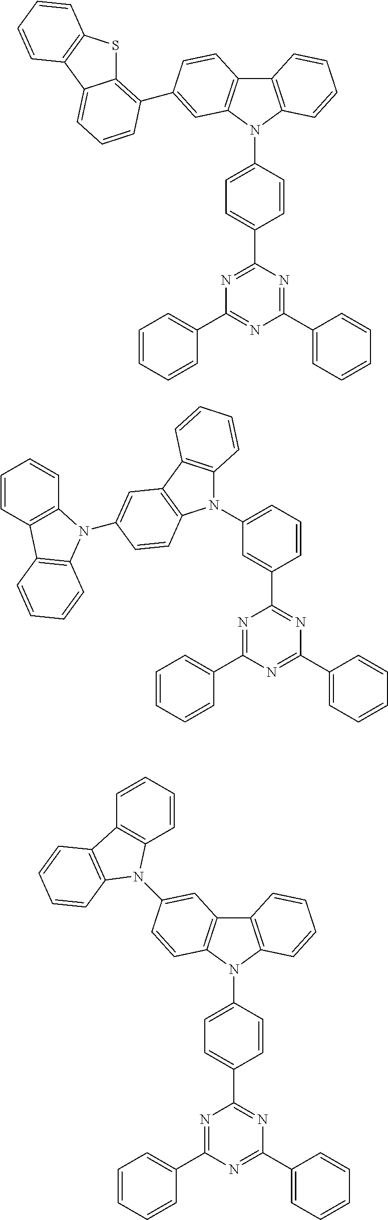

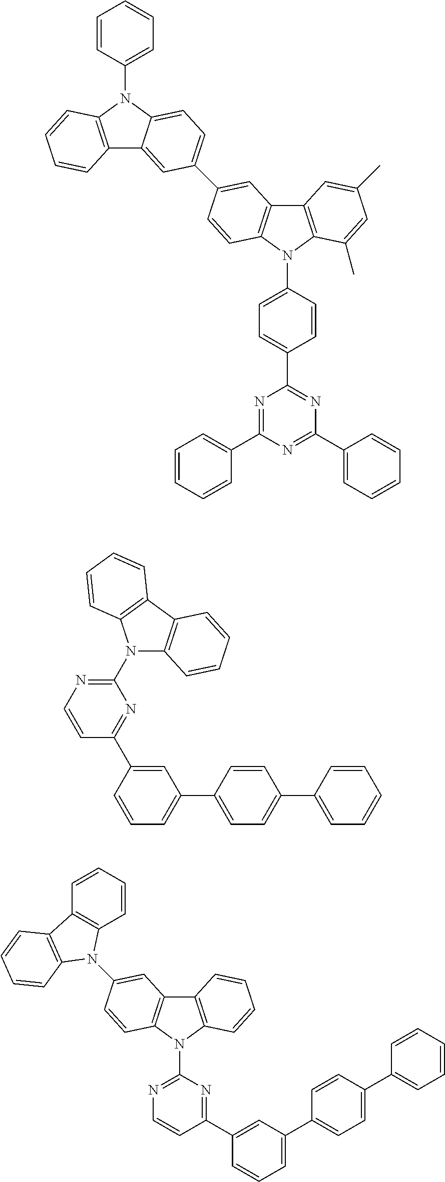 [SMILES: Cc1cc(C)c2c(c1)c1cc(-c3ccc4c(c3)c3ccccc3n4-c3ccccc3)ccc1n2-c1ccc(-c2nc(-c3ccccc3)nc(-c3ccccc3)n2)cc1 ; c1ccc(-c2ccc(-c3cccc(-c4ccnc(-n5c6ccccc6c6ccccc65)n4)c3)cc2)cc1 ; c1ccc(-c2ccc(-c3cccc(-c4ccnc(-n5c6ccccc6c6cc(-n7c8ccccc8c8ccccc87)ccc65)n4)c3)cc2)cc1]

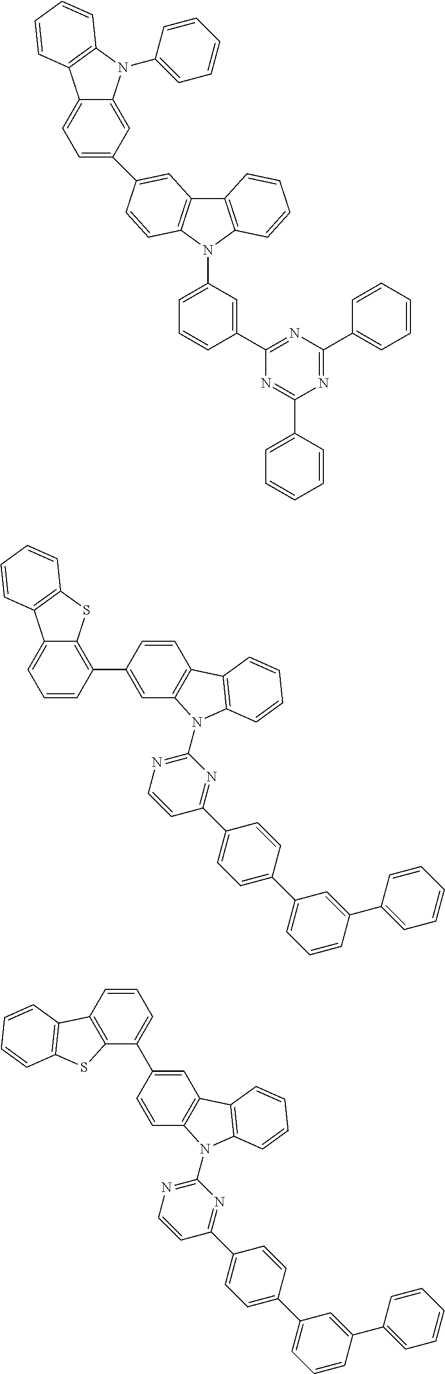 [SMILES: c1ccc(-c2nc(-c3ccccc3)nc(-c3cccc(-n4c5ccccc5c5cc(-c6ccc7c8ccccc8n(-c8ccccc8)c7c6)ccc54)c3)n2)cc1 ; c1ccc(-c2cccc(-c3ccc(-c4ccnc(-n5c6ccccc6c6ccc(-c7cccc8c7sc7ccccc78)cc65)n4)cc3)c2)cc1 ; c1ccc(-c2cccc(-c3ccc(-c4ccnc(-n5c6ccccc6c6cc(-c7cccc8c7sc7ccccc78)ccc65)n4)cc3)c2)cc1]

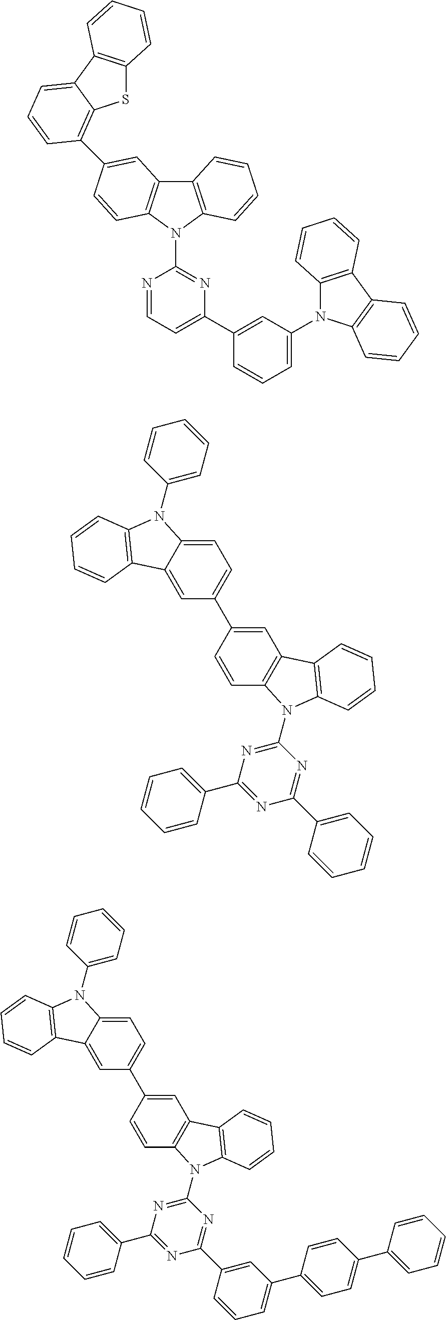 [SMILES: c1cc(-c2ccnc(-n3c4ccccc4c4cc(-c5cccc6c5sc5ccccc56)ccc43)n2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2ccc(-c3cccc(-c4nc(-c5ccccc5)nc(-n5c6ccccc6c6cc(-c7ccc8c(c7)c7ccccc7n8-c7ccccc7)ccc65)n4)c3)cc2)cc1]

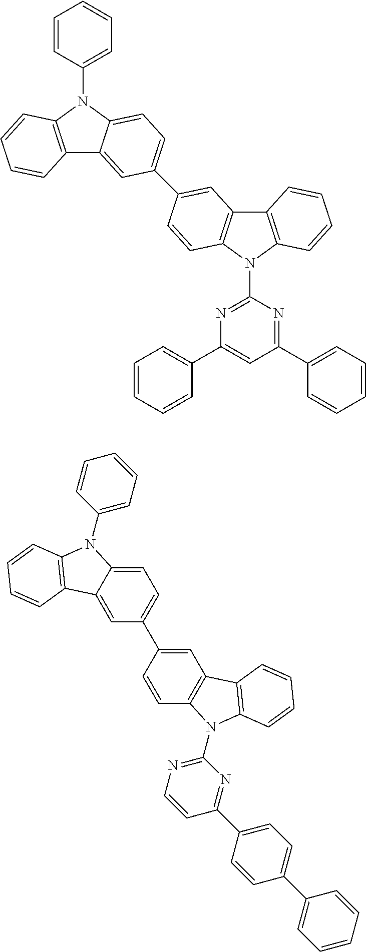 [SMILES: c1ccc(-c2cc(-c3ccccc3)nc(-n3c4ccccc4c4cc(-c5ccc6c(c5)c5ccccc5n6-c5ccccc5)ccc43)n2)cc1 ; c1ccc(-c2ccc(-c3ccnc(-n4c5ccccc5c5cc(-c6ccc7c(c6)c6ccccc6n7-c6ccccc6)ccc54)n3)cc2)cc1]

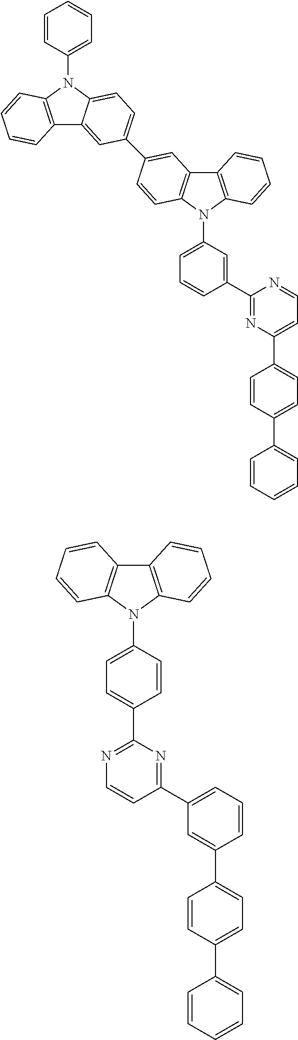 [SMILES: c1ccc(-c2ccc(-c3ccnc(-c4cccc(-n5c6ccccc6c6cc(-c7ccc8c(c7)c7ccccc7n8-c7ccccc7)ccc65)c4)n3)cc2)cc1 ; c1ccc(-c2ccc(-c3cccc(-c4ccnc(-c5ccc(-n6c7ccccc7c7ccccc76)cc5)n4)c3)cc2)cc1]

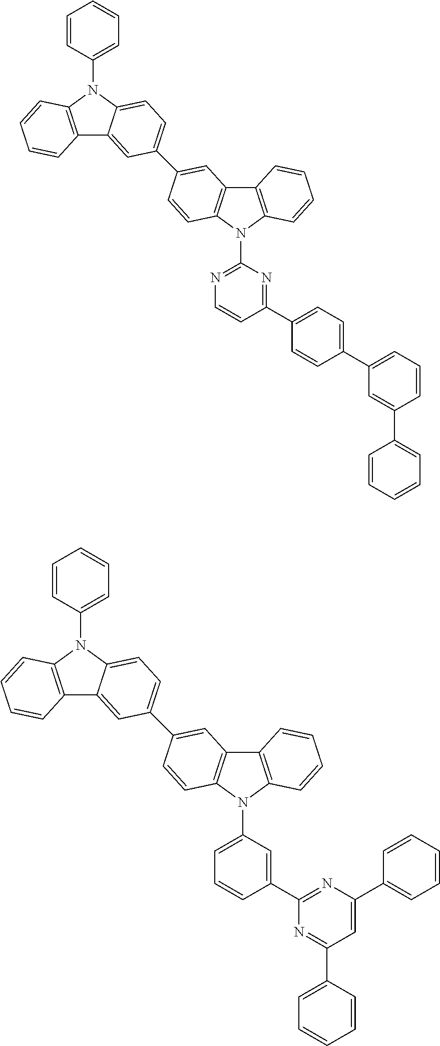 [SMILES: c1ccc(-c2cccc(-c3ccc(-c4ccnc(-n5c6ccccc6c6cc(-c7ccc8c(c7)c7ccccc7n8-c7ccccc7)ccc65)n4)cc3)c2)cc1 ; c1ccc(-c2cc(-c3ccccc3)nc(-c3cccc(-n4c5ccccc5c5cc(-c6ccc7c(c6)c6ccccc6n7-c6ccccc6)ccc54)c3)n2)cc1]

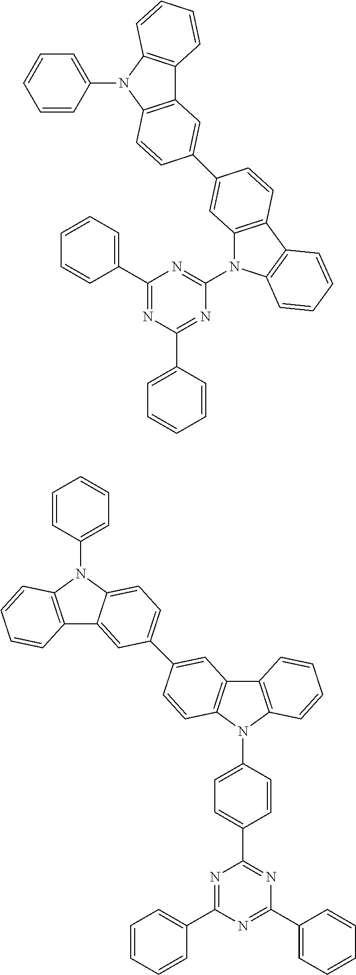 [SMILES: c1ccc(-c2nc(-c3ccccc3)nc(-n3c4ccccc4c4ccc(-c5ccc6c(c5)c5ccccc5n6-c5ccccc5)cc43)n2)cc1 ; c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1]

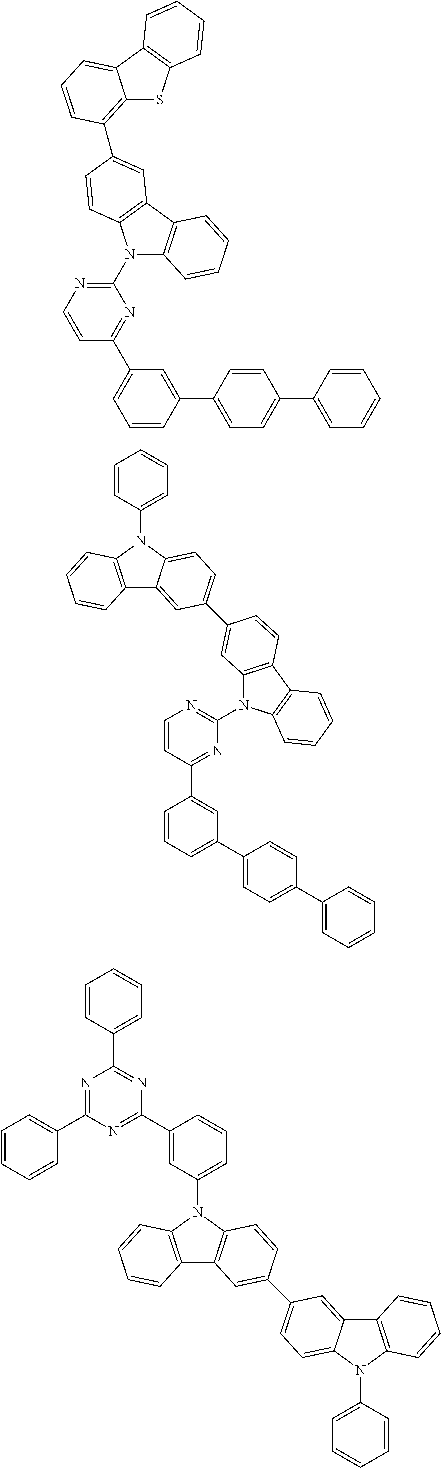

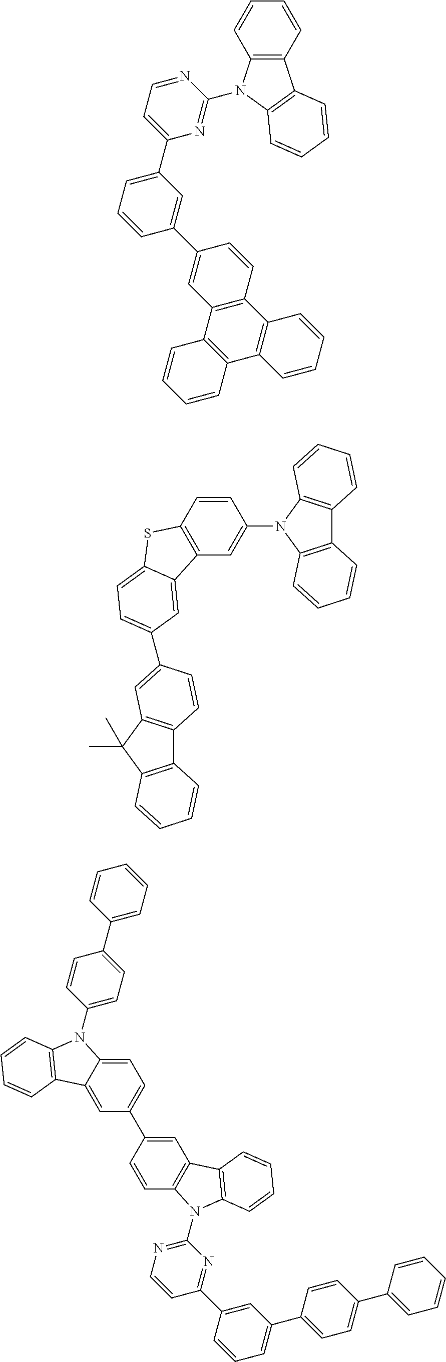 [SMILES: c1cc(-c2ccc3c4ccccc4c4ccccc4c3c2)cc(-c2ccnc(-n3c4ccccc4c4ccccc43)n2)c1 ; CC1(C)c2ccccc2-c2ccc(-c3ccc4sc5ccc(-n6c7ccccc7c7ccccc76)cc5c4c3)cc21 ; c1ccc(-c2ccc(-c3cccc(-c4ccnc(-n5c6ccccc6c6cc(-c7ccc8c(c7)c7ccccc7n8-c7ccc(-c8ccccc8)cc7)ccc65)n4)c3)cc2)cc1]

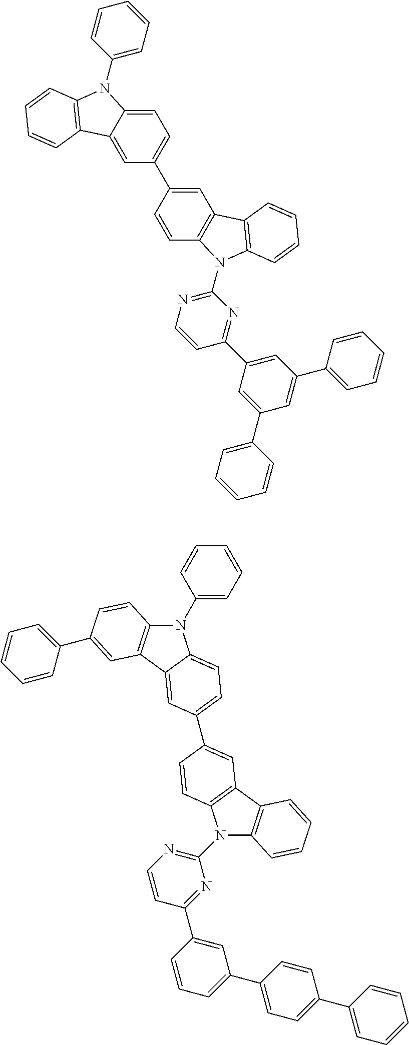 [SMILES: c1ccc(-c2cc(-c3ccccc3)cc(-c3ccnc(-n4c5ccccc5c5cc(-c6ccc7c(c6)c6ccccc6n7-c6ccccc6)ccc54)n3)c2)cc1 ; c1ccc(-c2ccc(-c3cccc(-c4ccnc(-n5c6ccccc6c6cc(-c7ccc8c(c7)c7cc(-c9ccccc9)ccc7n8-c7ccccc7)ccc65)n4)c3)cc2)cc1]

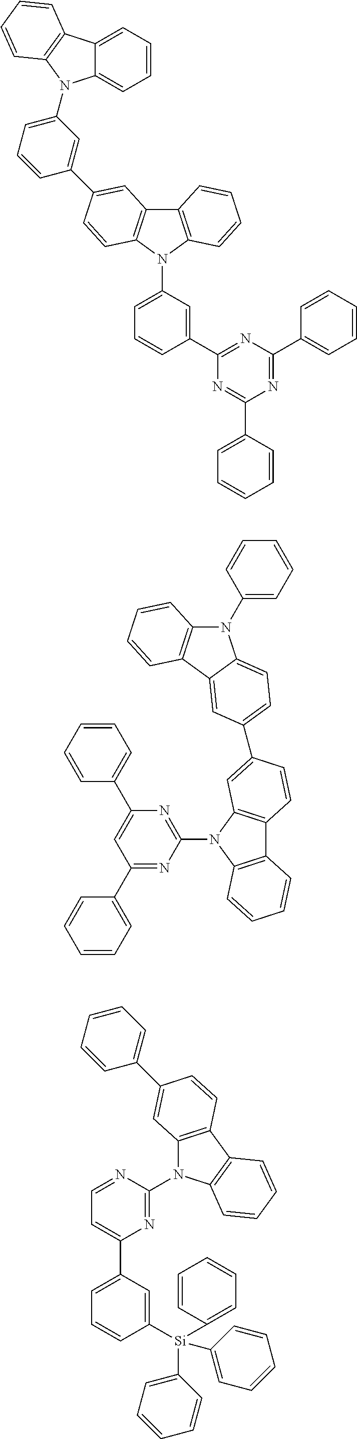

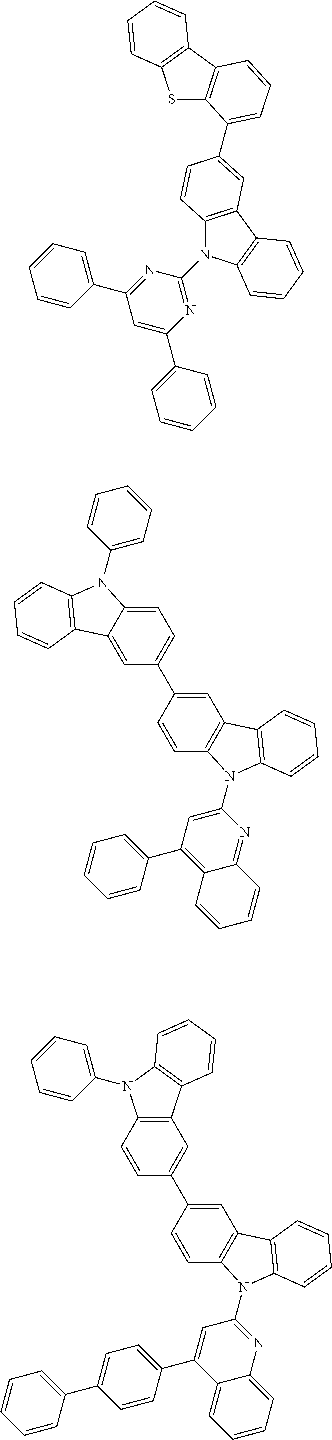 [SMILES: c1ccc(-c2cc(-c3ccccc3)nc(-n3c4ccccc4c4cc(-c5cccc6c5sc5ccccc56)ccc43)n2)cc1 ; c1ccc(-c2cc(-n3c4ccccc4c4cc(-c5ccc6c(c5)c5ccccc5n6-c5ccccc5)ccc43)nc3ccccc23)cc1 ; c1ccc(-c2ccc(-c3cc(-n4c5ccccc5c5cc(-c6ccc7c(c6)c6ccccc6n7-c6ccccc6)ccc54)nc4ccccc34)cc2)cc1]

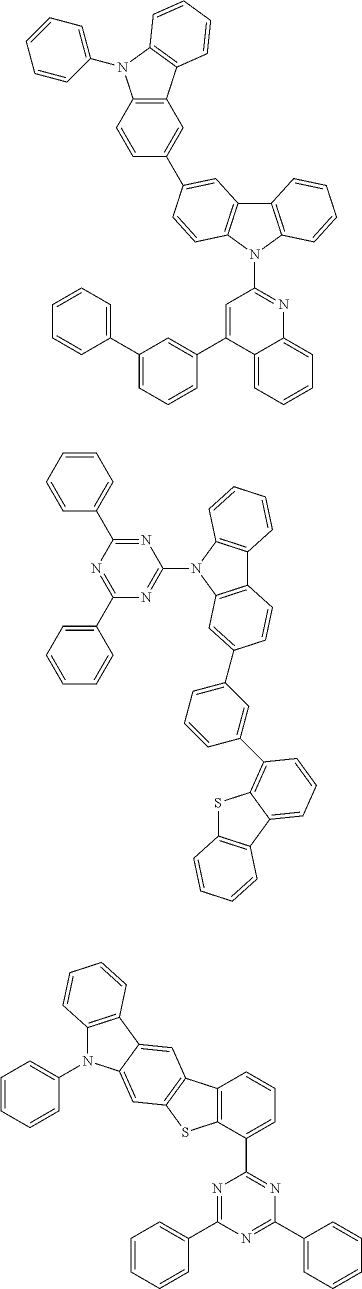 [SMILES: c1ccc(-c2cccc(-c3cc(-n4c5ccccc5c5cc(-c6ccc7c(c6)c6ccccc6n7-c6ccccc6)ccc54)nc4ccccc34)c2)cc1 ; c1ccc(-c2nc(-c3ccccc3)nc(-n3c4ccccc4c4ccc(-c5cccc(-c6cccc7c6sc6ccccc67)c5)cc43)n2)cc1 ; c1ccc(-c2nc(-c3ccccc3)nc(-c3cccc4c3sc3cc5c(cc34)c3ccccc3n5-c3ccccc3)n2)cc1]

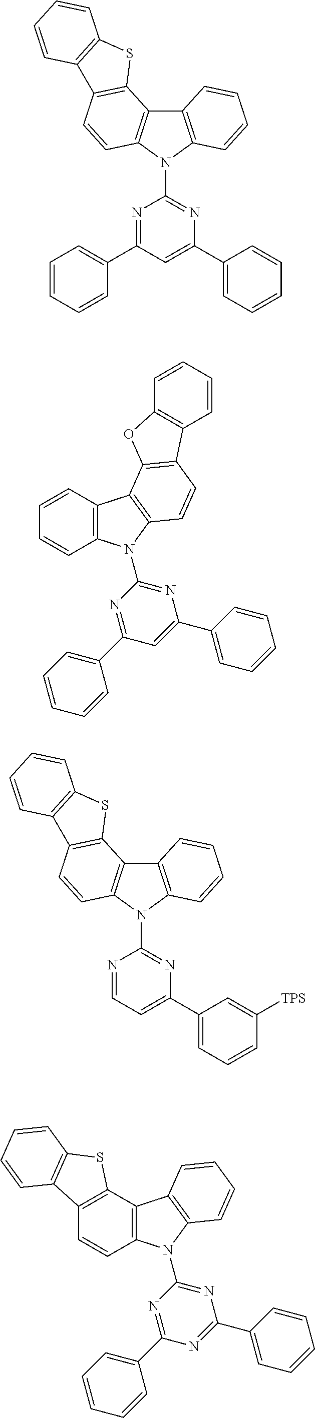

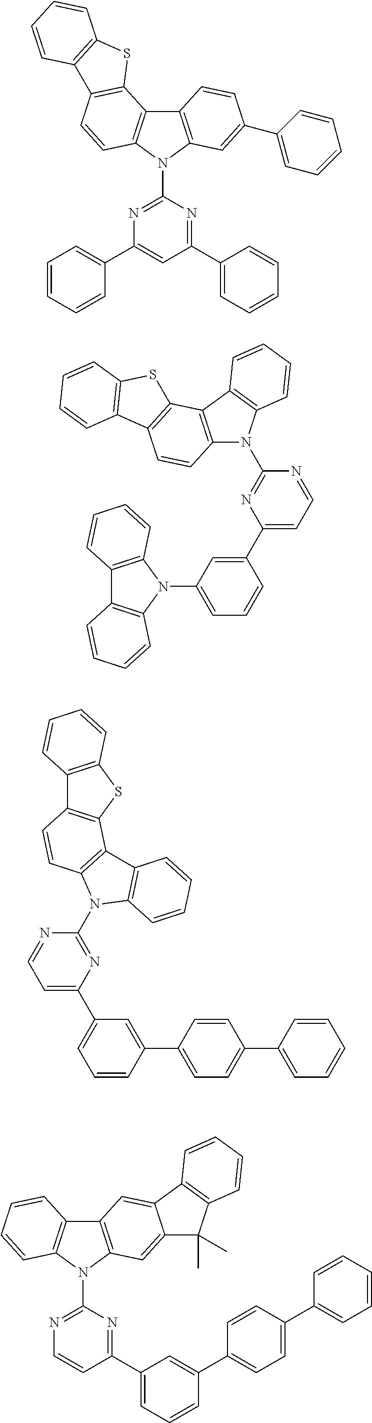

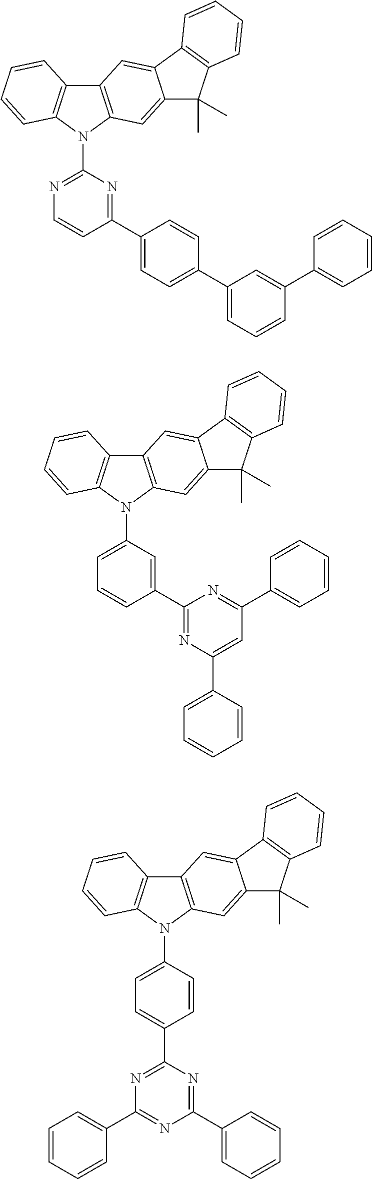

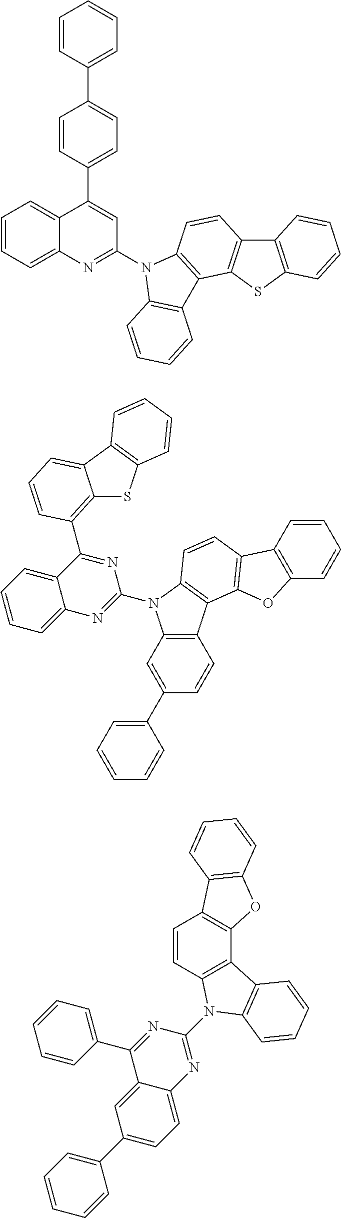

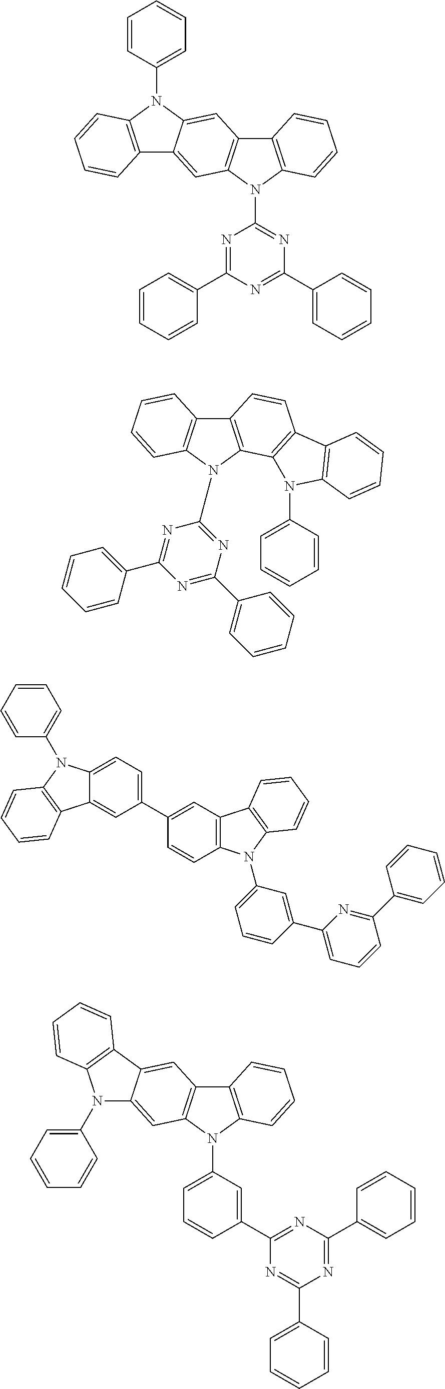 [SMILES: c1ccc(-c2nc(-c3ccccc3)nc(-n3c4ccccc4c4cc5c(cc43)c3ccccc3n5-c3ccccc3)n2)cc1 ; c1ccc(-c2nc(-c3ccccc3)nc(-n3c4ccccc4c4ccc5c6ccccc6n(-c6ccccc6)c5c43)n2)cc1 ; c1ccc(-c2cccc(-c3cccc(-n4c5ccccc5c5cc(-c6ccc7c(c6)c6ccccc6n7-c6ccccc6)ccc54)c3)n2)cc1 ; c1ccc(-c2nc(-c3ccccc3)nc(-c3cccc(-n4c5ccccc5c5cc6c7ccccc7n(-c7ccccc7)c6cc54)c3)n2)cc1]

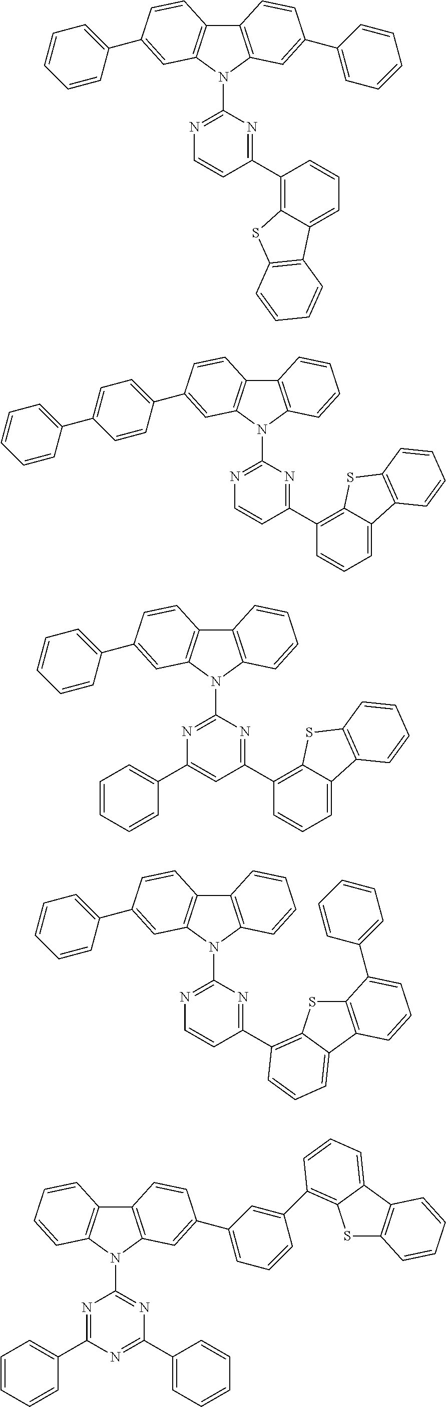

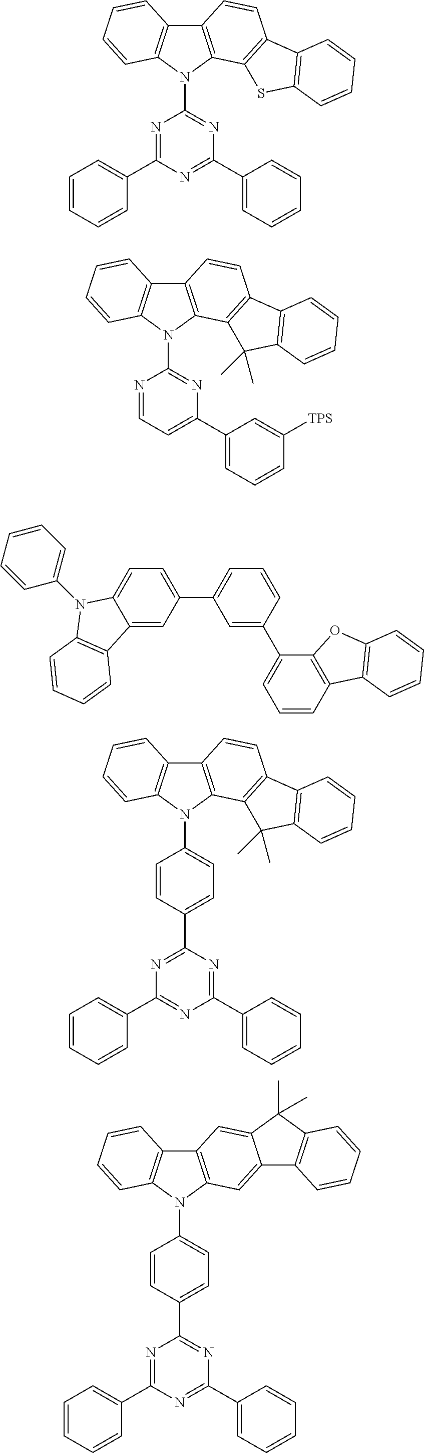

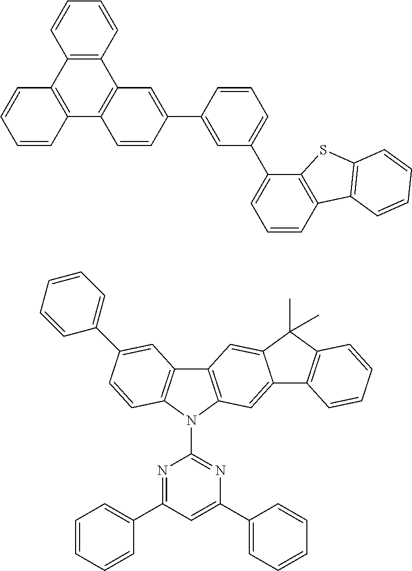 [SMILES: c1cc(-c2ccc3c4ccccc4c4ccccc4c3c2)cc(-c2cccc3c2sc2ccccc23)c1 ; CC1(C)c2ccccc2-c2cc3c(cc21)c1cc(-c2ccccc2)ccc1n3-c1nc(-c2ccccc2)cc(-c2ccccc2)n1]

The dopant comprised in the organic electroluminescent device according to the present invention may be preferably selected from metallated complex compounds of iridium, copper and platinum; more preferably selected from ortho-metallated complex compounds of iridium, copper and platinum; and even more preferably ortho-metallated iridium complex compounds.

The dopants may be selected from compounds represented by the following formulas 10 to 12.

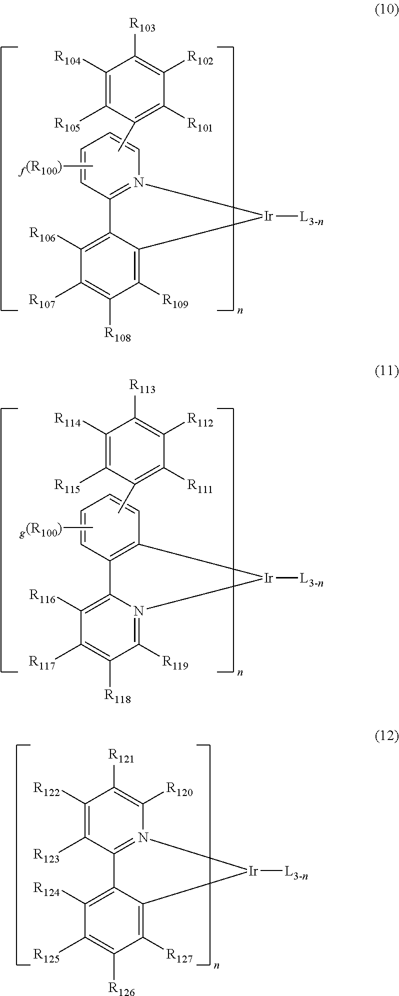

wherein L is selected from the following structures:

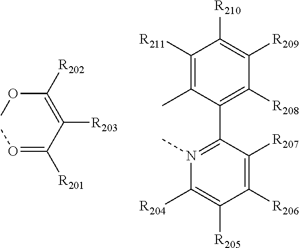

R₁₀₀ represents hydrogen, a substituted or unsubstituted (C1-C30)alkyl group, or a substituted or unsubstituted (C3-C30)cycloalkyl group;

R₁₀₁ to R₁₀₉, and R₁₁₁ to R₁₂₃ each independently represent hydrogen, deuterium, a halogen, a (C1-C30)alkyl group unsubstituted or substituted with halogen(s), a substituted or unsubstituted (C3-C30)cycloalkyl group, a cyano group, or a substituted or unsubstituted (C1-C30)alkoxy group; adjacent substituents of R₁₂₀ to R₁₂₃ may be linked to each other to form a fused ring, e.g. substituted or unsubstituted quinoline;

R₁₂₄ to R₁₂₇ each independently represent hydrogen, deuterium, a halogen, a substituted or unsubstituted (C1-C30)alkyl group, or a substituted or unsubstituted (C6-C30)aryl group; where R₁₂₄ to R₁₂₇ are aryl groups, adjacent substituents may be linked to each other to form a fused ring, e.g. substituted or unsubstituted fluorene;

R₂₀₁ to R₂₁₁ each independently represent hydrogen, deuterium, a halogen, a (C1-C30)alkyl group unsubstituted or substituted with halogen(s), or a substituted or unsubstituted (C3-C30)cycloalkyl group;

f and g each independently represent an integer of 1 to 3; where f or g is an integer of 2 or more, each of R₁₀₀ may be the same or different; and

n is an integer of 1 to 3.

Specifically, the dopant materials include the following:

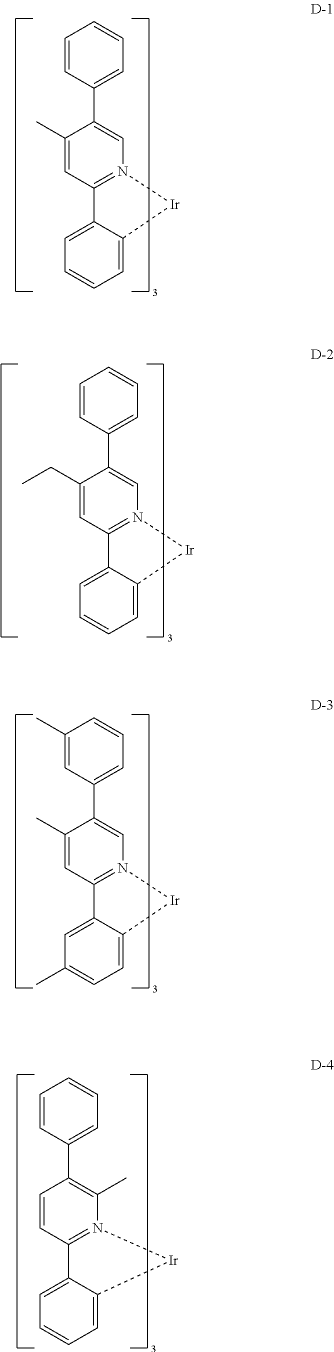

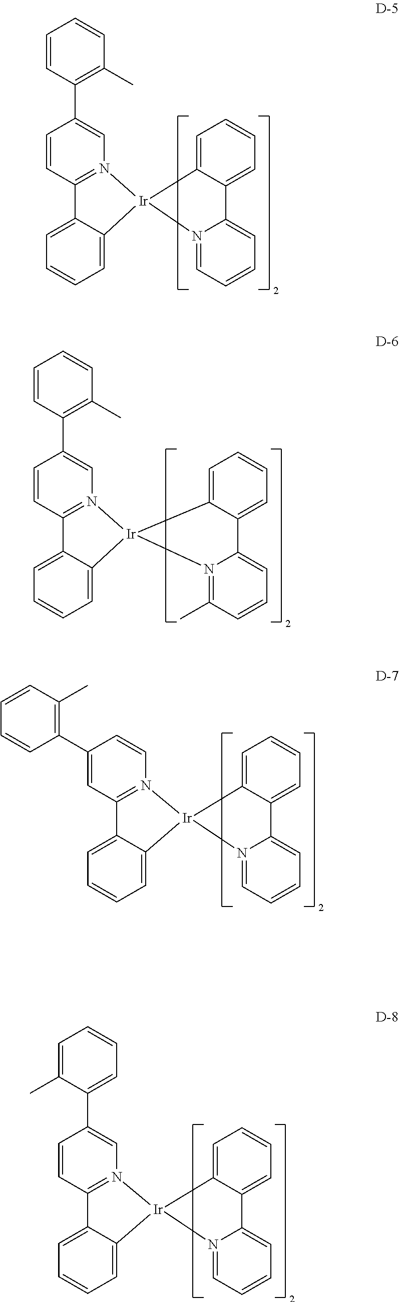

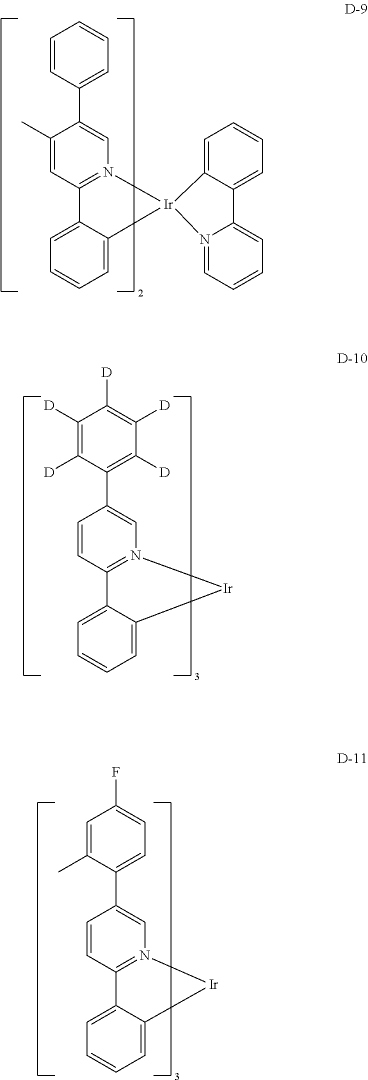

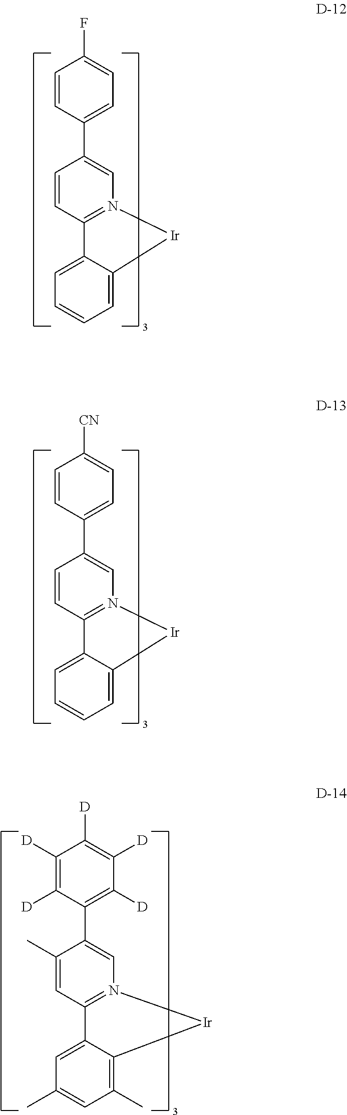

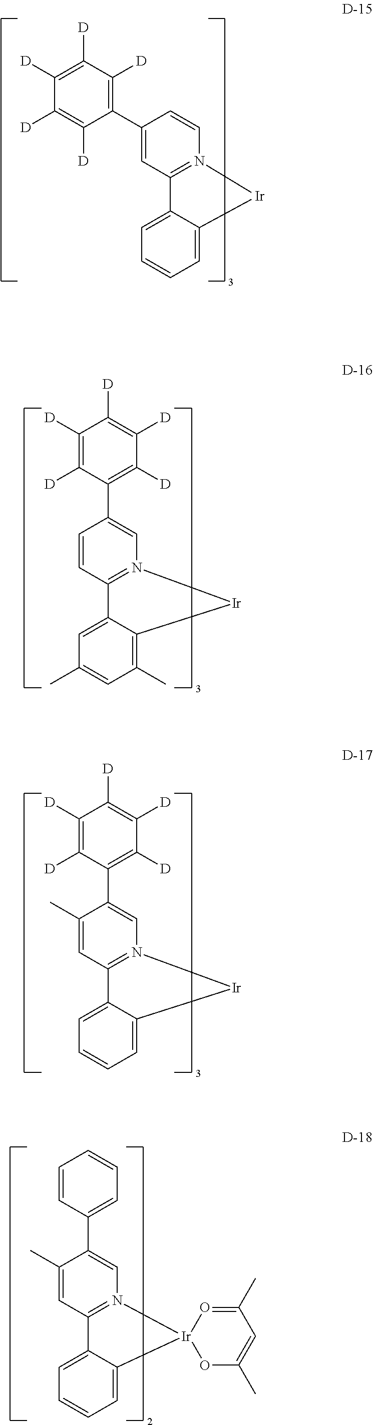

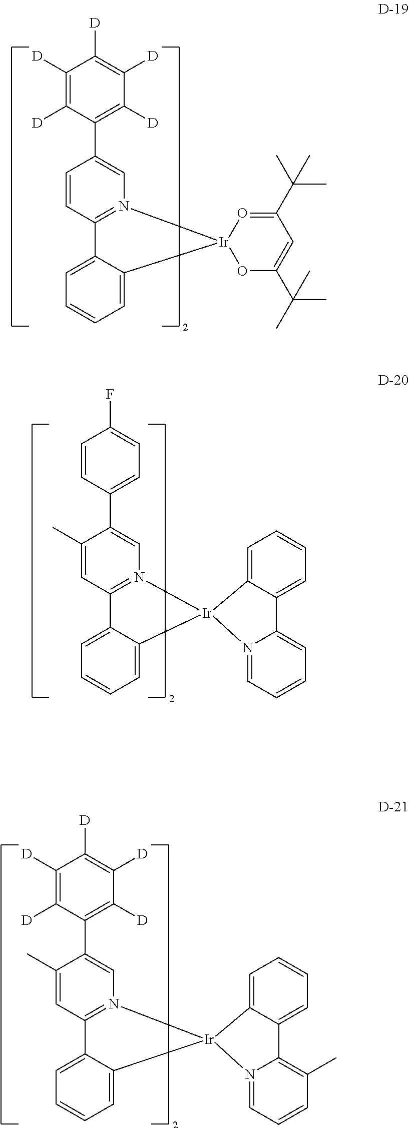

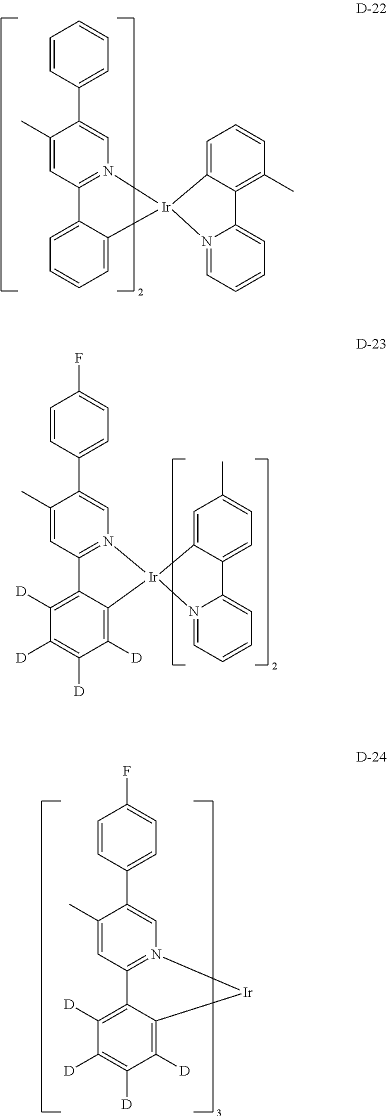

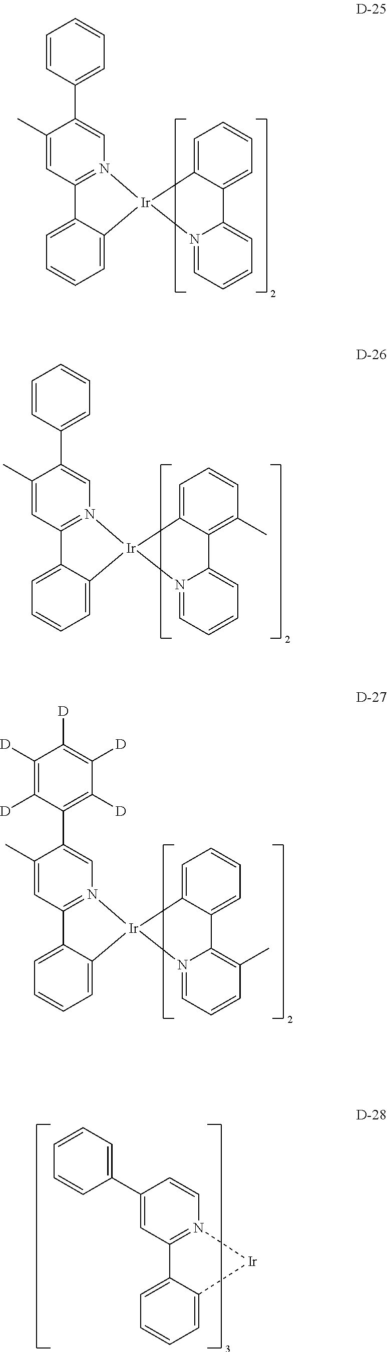

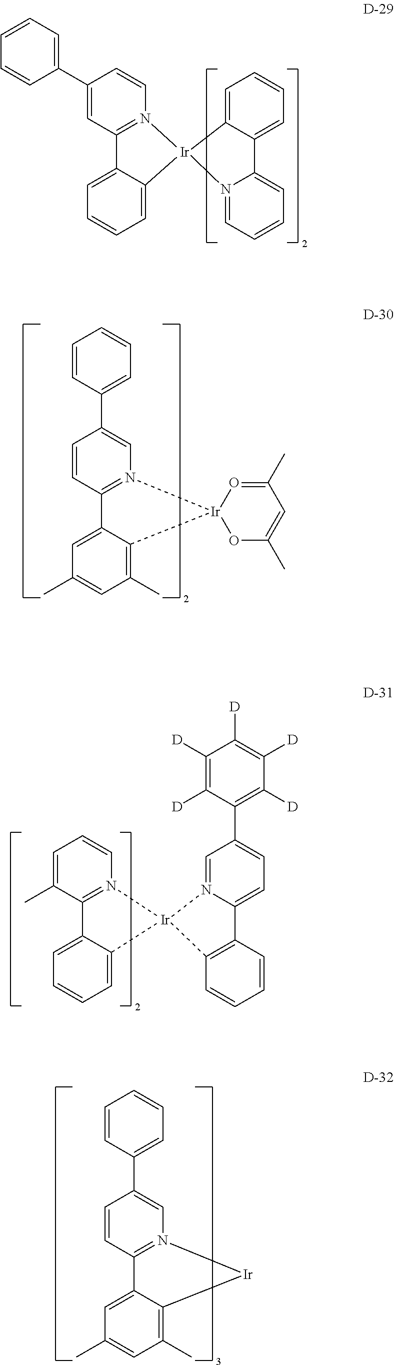

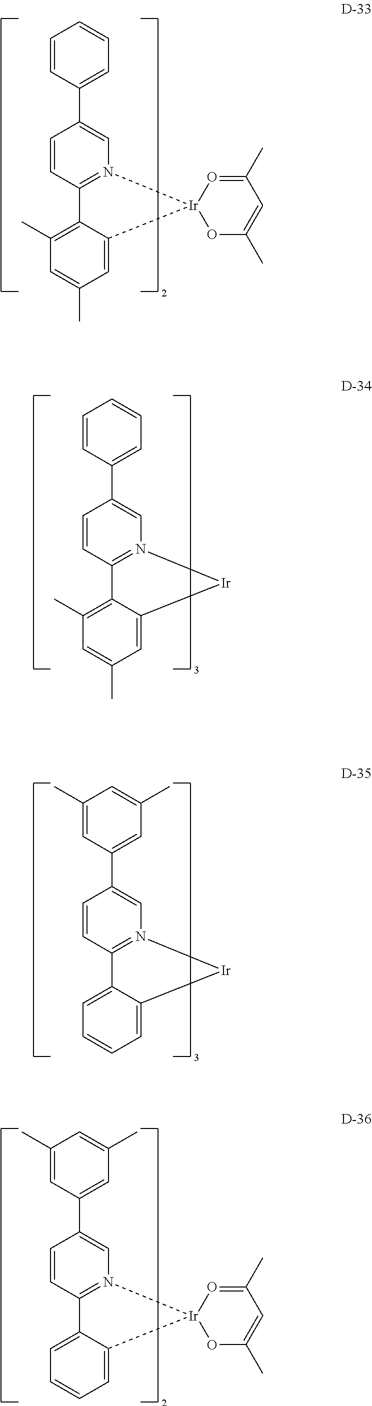

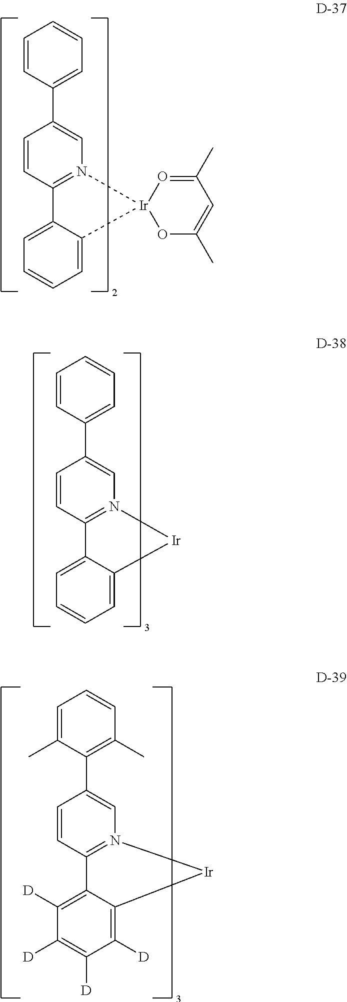

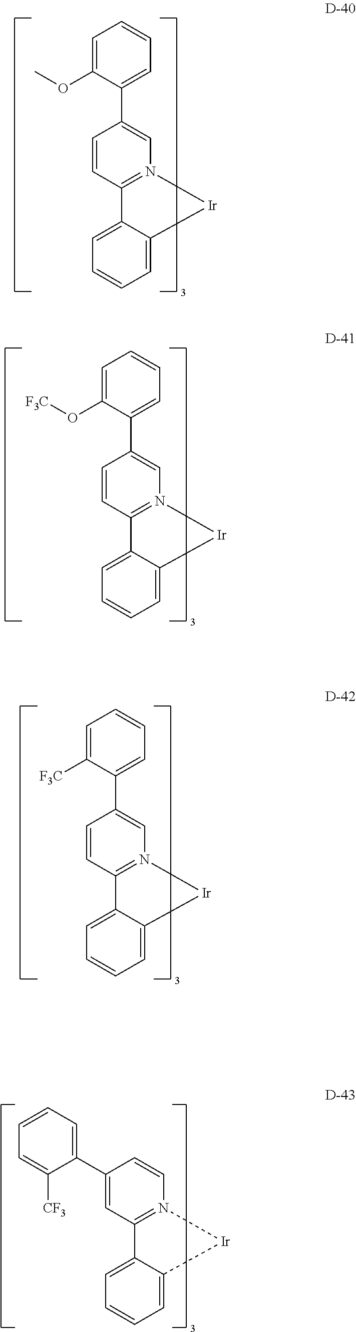

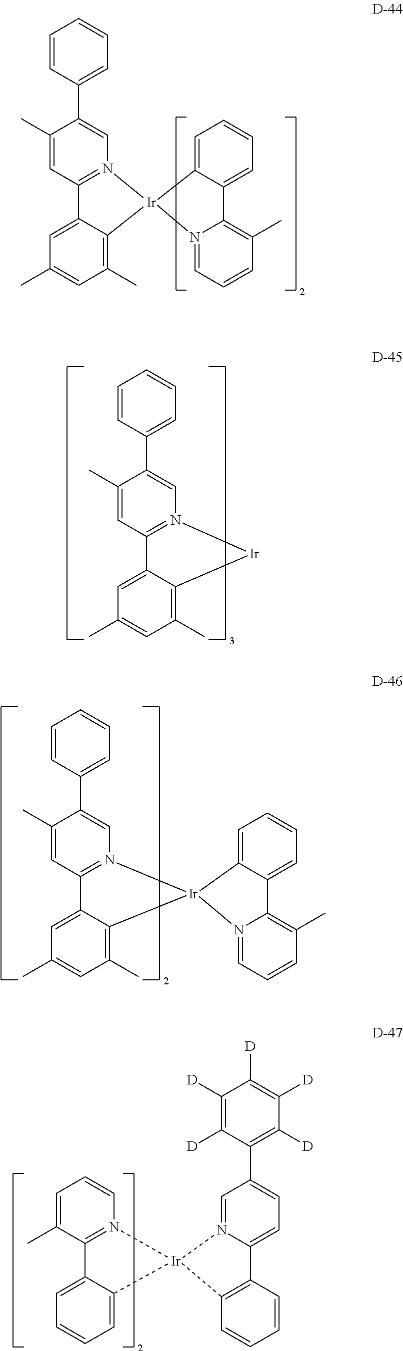

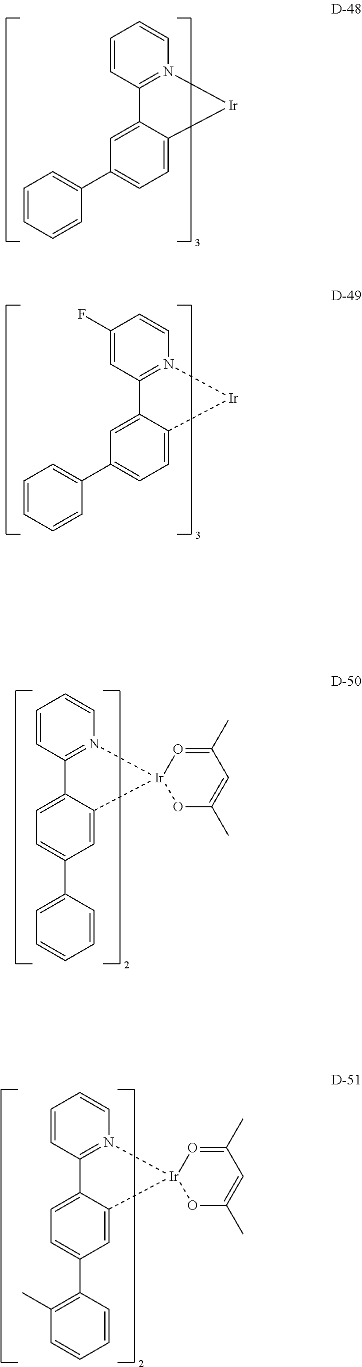

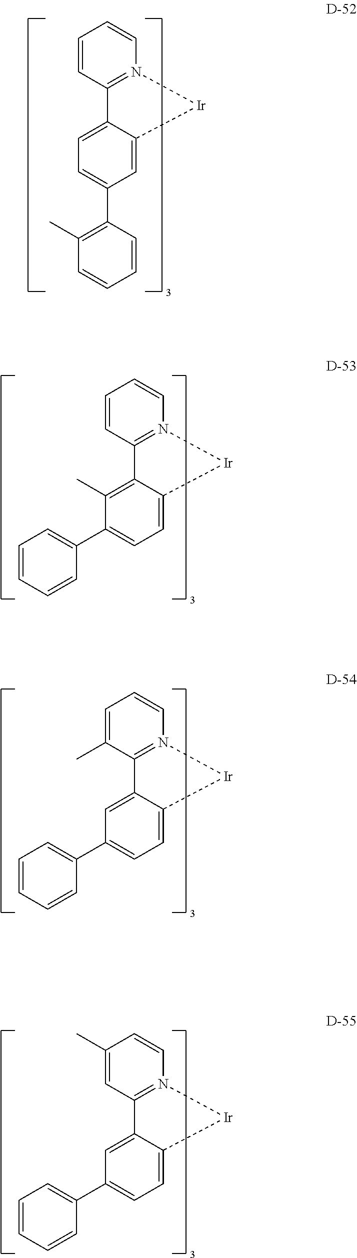

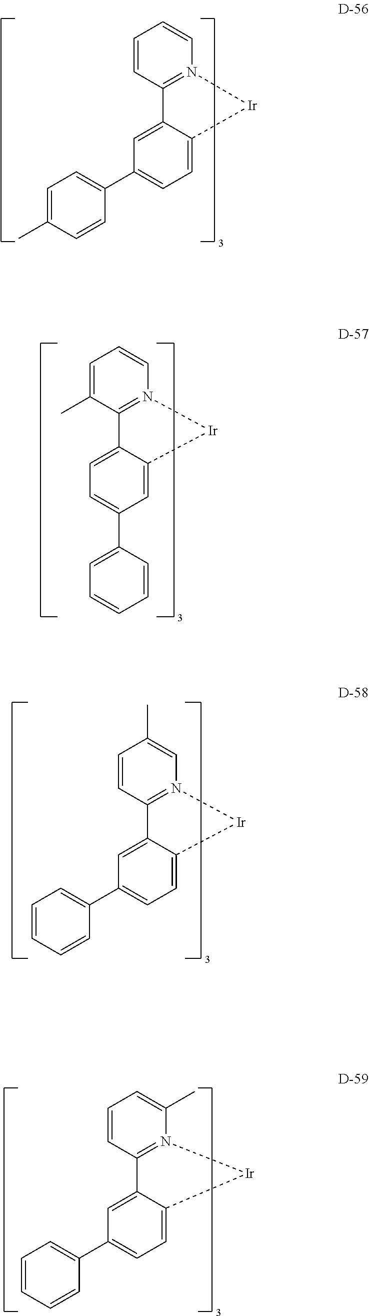

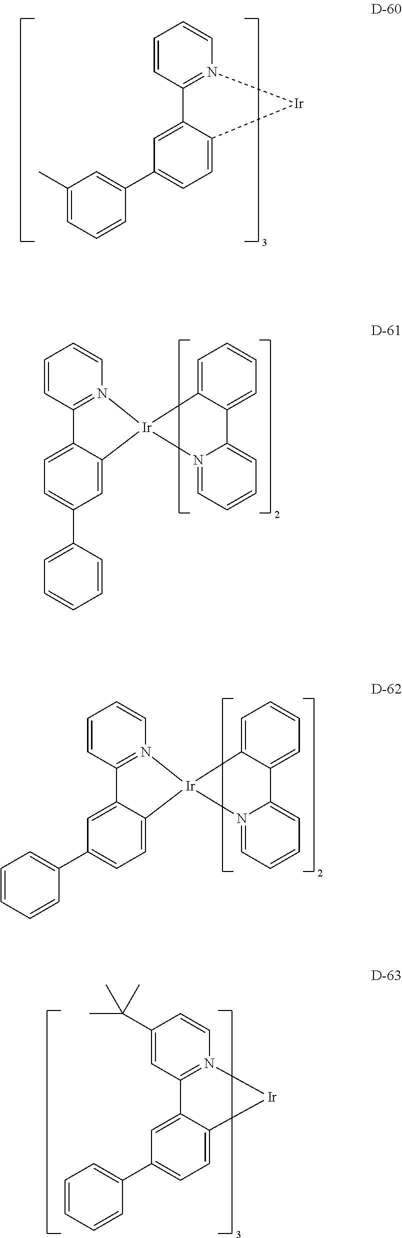

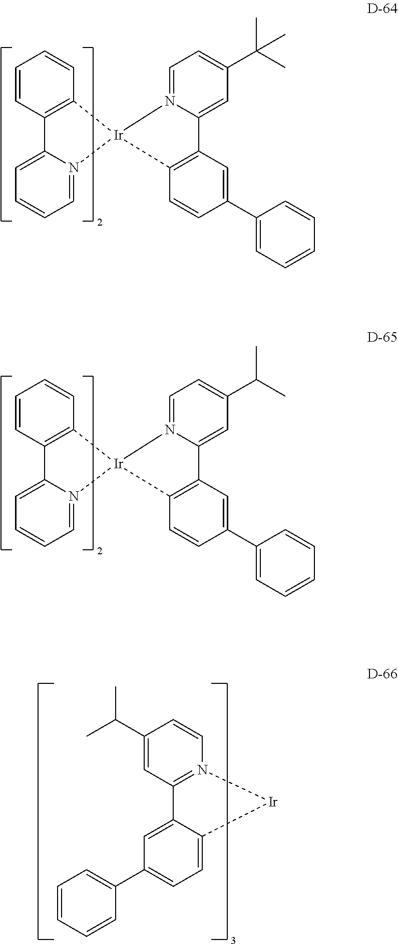

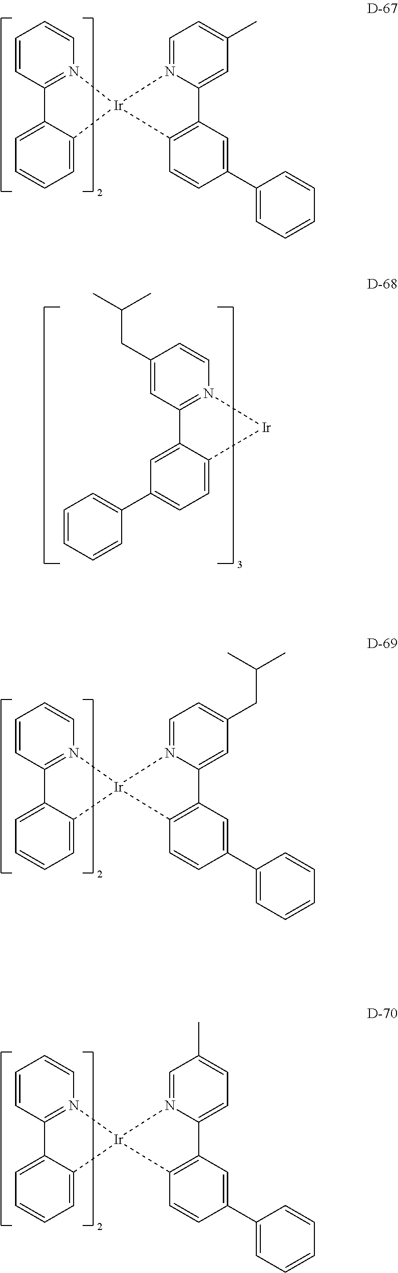

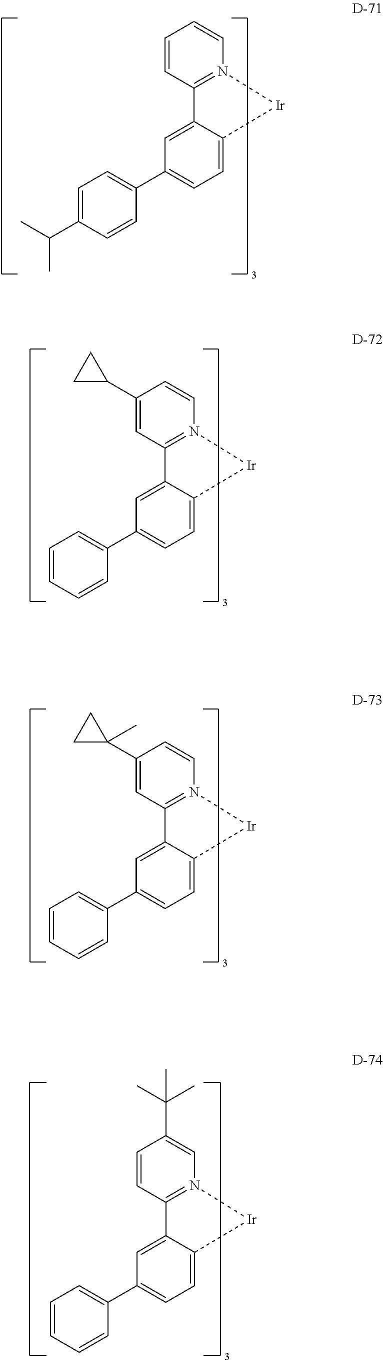

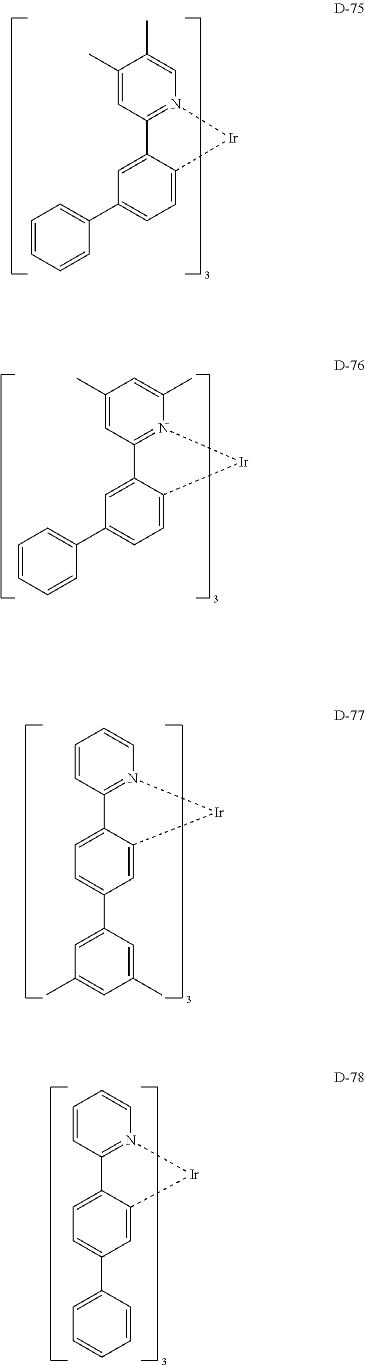

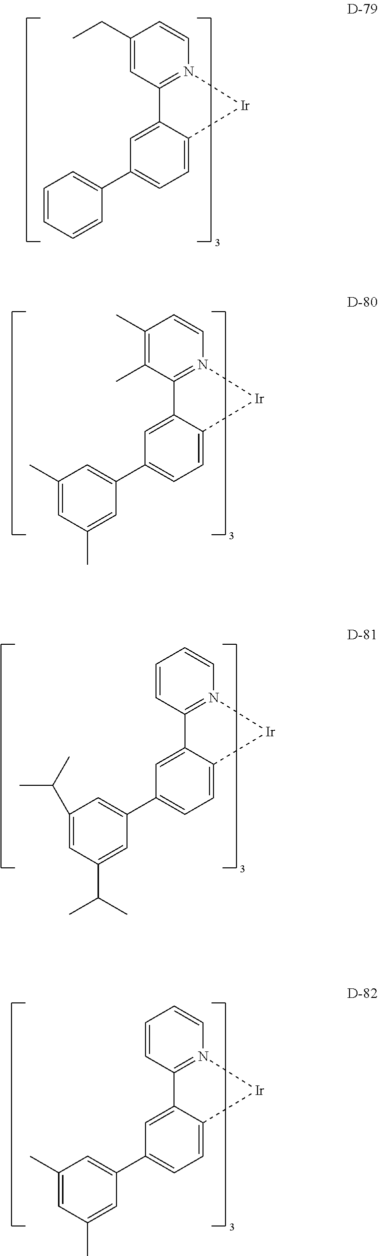

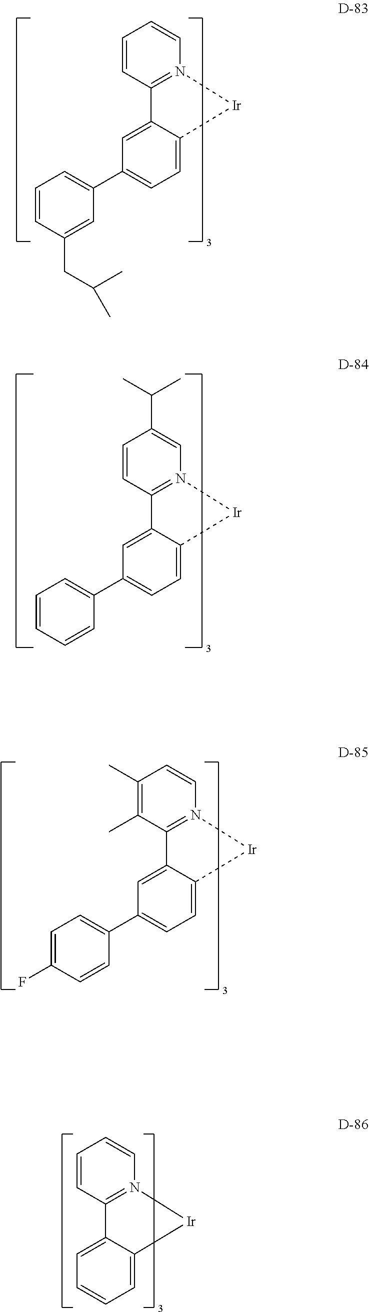

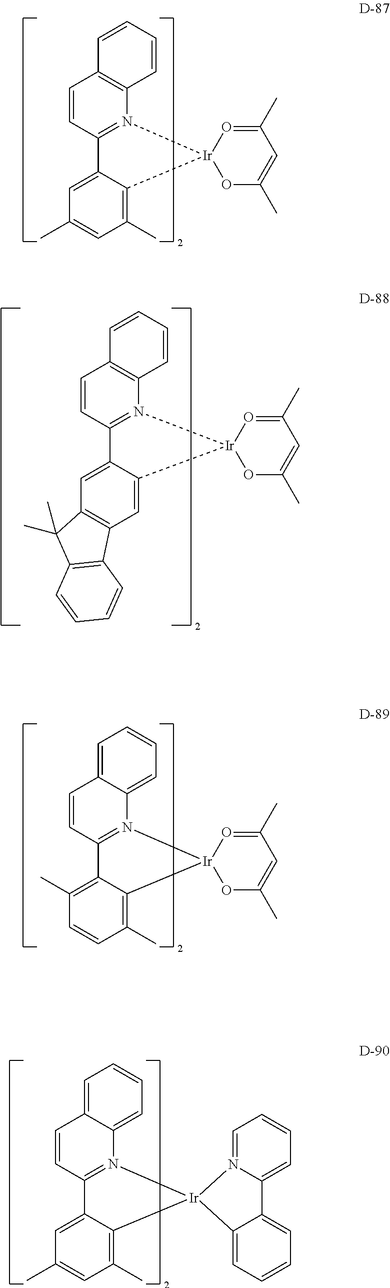

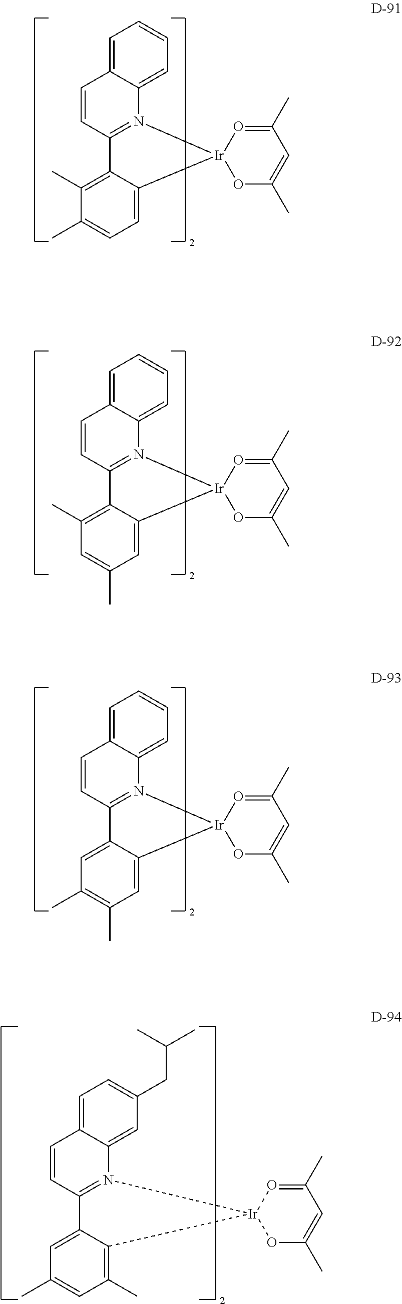

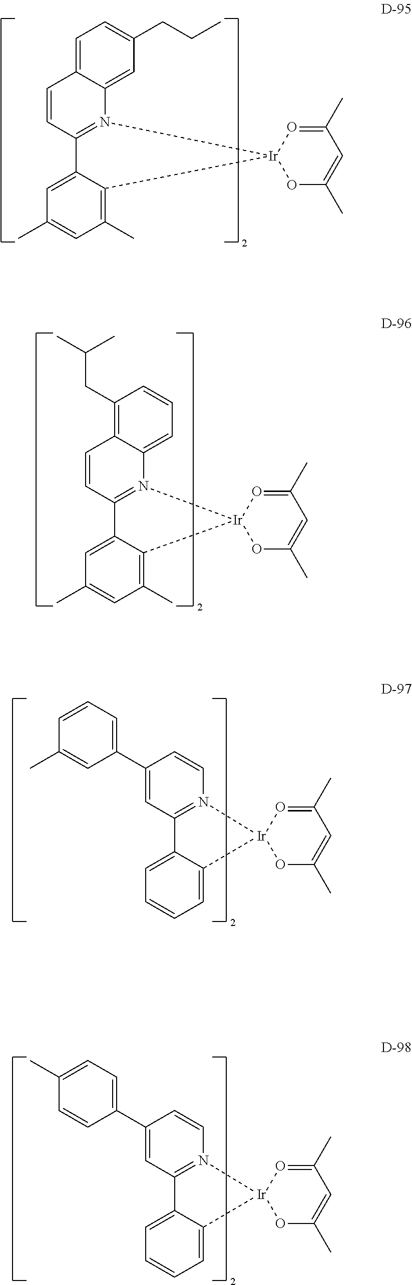

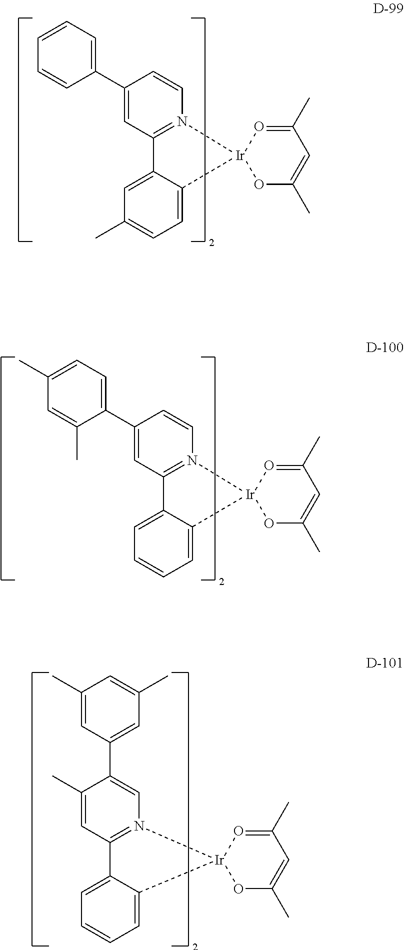

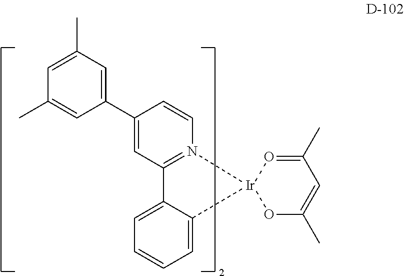

In another embodiment of the present invention, a material used for an organic electroluminescent device is provided. The material comprises the compound according to the present invention as a host material. When the compound according to the present invention is comprised as a host material (first host material), another compound can be comprised in the material used for an organic electroluminescent device, as a second host material, wherein the ratio of the first host material to the second host material can be in the range of 1:99 to 99:1.

In addition, the organic electroluminescent device according to the present invention comprises a first electrode, a second electrode, and at least one organic layer between said first and second electrodes. Said organic layer may comprise a material used for an organic electroluminescent device according to the present invention.

The organic electroluminescent device according to the present invention may further comprise, in addition to the organic electroluminescent compounds represented by formula 1, at least one compound selected from the group consisting of arylamine-based compounds and styrylarylamine-based compounds.

In the organic electroluminescent device according to the present invention, the organic layer may further comprise at least one metal selected from the group consisting of metals of Group 1, metals of Group 2, transition metals of the 4^{th} period, transition metals of the 5^{th} period, lanthanides and organic metals of d-transition elements of the Periodic Table, or at least one complex compound comprising said metal. The organic layer may further comprise a charge generating layer.

In addition, the organic electroluminescent device according to the present invention may emit white light on the whole by further comprising at least one light-emitting layer which comprises a blue electroluminescent compound, a red electroluminescent compound or a green electroluminescent compound known in the field, besides the organic electroluminescent compound according to the present invention. Also, if needed, a yellow or orange light-emitting layer can be comprised in the device.

According to the present invention, at least one layer (hereinafter, “a surface layer”) may be preferably placed on an inner surface(s) of one or both electrode(s); selected from a chalcogenide layer, a metal halide layer and a metal oxide layer. Specifically, a chalcogenide(includes oxides) layer of silicon or aluminum is preferably placed on an anode surface of an electroluminescent medium layer, and a metal halide layer or a metal oxide layer is preferably placed on a cathode surface of an electroluminescent medium layer. Such a surface layer provides operation stability for the organic electroluminescent device. Preferably, said chalcogenide includes SiOₓ(1≦X≦2), AlOₓ(1≦X≦1.5), SiON, SiAlON, etc.; said metal halide includes LiF, MgF₂, CaF₂, a rare earth metal fluoride, etc.; and said metal oxide includes Cs₂O, Li₂O, MgO, SrO, BaO, CaO, etc.

Preferably, in the organic electroluminescent device according to the present invention, a mixed region of an electron transport compound and an reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to an electroluminescent medium. Further, the hole transport compound is oxidized to a cation, and thus it becomes easier to inject and transport holes from the mixed region to the electroluminescent medium. Preferably, the oxidative dopant includes various Lewis acids and acceptor compounds; and the reductive dopant includes alkali metals, alkali metal compounds, alkaline earth metals, rare-earth metals, and mixtures thereof. A reductive dopant layer may be employed as a charge generating layer to prepare an electroluminescent device having two or more electroluminescent layers and emitting white light.

In order to form each layer of the organic electroluminescent device according to the present invention, dry film-forming methods such as vacuum evaporation, sputtering, plasma and ion plating methods, or wet film-forming methods such as spin coating, dip coating, flow coating methods can be used.

When using a wet film-forming method, a thin film can be formed by dissolving or diffusing materials forming each layer into any suitable solvent such as ethanol, chloroform, tetrahydrofuran, dioxane, etc. The solvent can be any solvent where the materials forming each layer can be dissolved or diffused, and where there are no problems in film-formation capability.

Hereinafter, the organic electroluminescent compound, the preparation method of the compound, and the luminescent properties of the device comprising the compound of the present invention will be explained in detail with reference to the following examples.

### EXAMPLE 1

### Preparation of Compound C-25

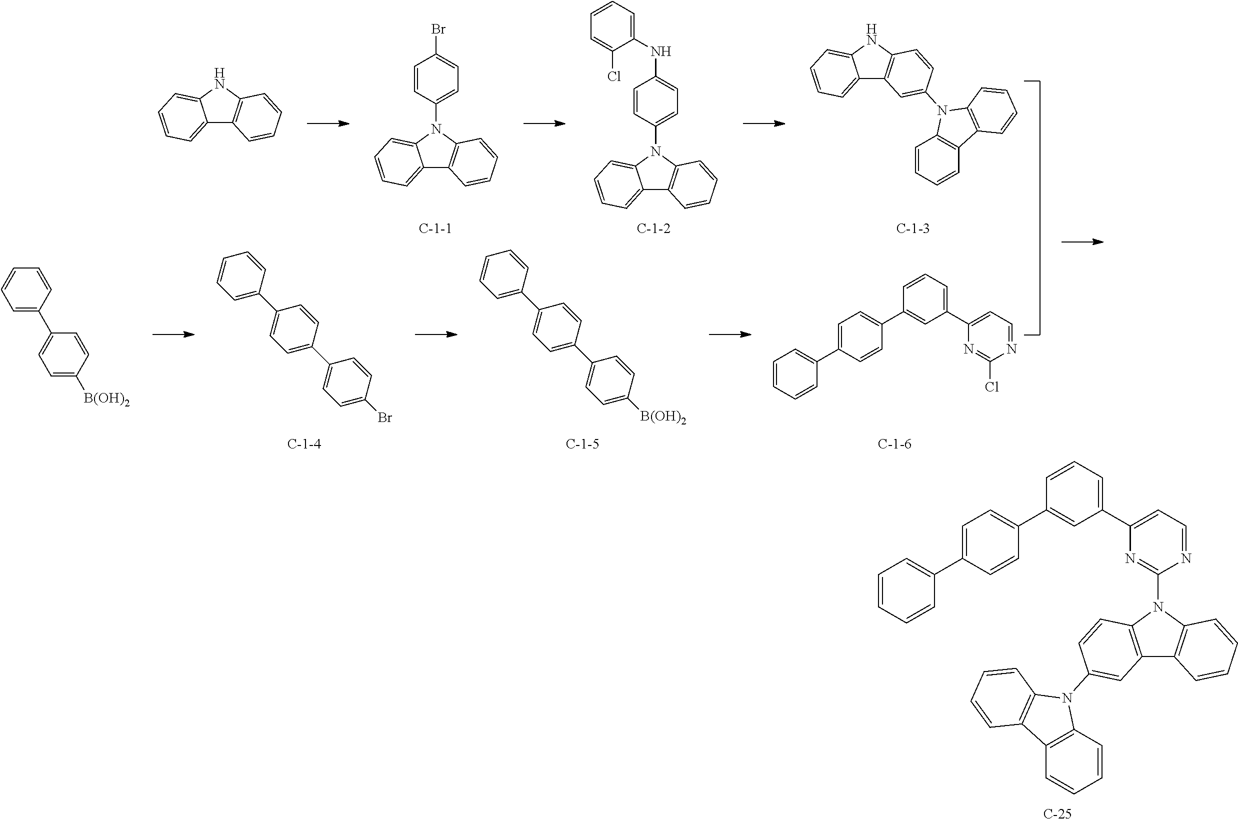

### Preparation of Compound C-1-1

After dissolving carbazole (25 g, 149.5 mmol), 1-iodo-4-bromobenzene (126 g, 448.5 mmol), CuI (14.2 g, 74.7 mmol), ethylenediamine (5 mL, 74.7 mmol), and K₃PO₄ (95 g, 448.5 mmol) in toluene 450 mL, the mixture was stirred under reflux for 24 hours at 120° C. After the reaction was completed, an organic layer was extracted with ethylacetate (EA) from the mixture; the remaining moisture was removed using magnesium sulfate; dried; and then the remaining product was separated with a column to obtain compound C-1-1 (42 g, 85%).

### Preparation of Compound C-1-2

After dissolving compound C-1-1 (25 g, 77.6 mmol), 2-chloroaniline (24.4 mL, 232.7 mmol), palladium acetate (697 mg, 3.1 mmol), tri-t-butyl phosphine (1.53 mL, 6.2 mmol), and potassium t-butoxide (21.7 g, 193.9 mmol) in toluene 215 mL; the mixture was stirred under reflux for 24 hours at 120° C. After the reaction was completed, an organic layer was extracted with ethylacetate (EA) from the mixture; the remaining moisture was removed using magnesium sulfate; dried; and then the remaining product was separated with a column to obtain compound C-1-2 (19 g, 73%).

### Preparation of Compound C-1-3

After dissolving compound C-1-2 (19 g, 56.8 mmol), palladium acetate (638 mg, 2.84 mmol), tri-t-butyl phosphonium tetrafluoroborate (1.64 g, 5.68 mmol), and K₂CO₃ (23.5 g, 170.4 mmol) in dimethylacetamide (DMA); the mixture was stirred under reflux for 24 hours at 180° C. After the reaction was completed, an organic layer was extracted with ethylacetate (EA) from the mixture; the remaining moisture was removed using magnesium sulfate; dried; and then the remaining product was separated with a column to obtain compound C-1-3 (15 g, 80%).

### Preparation of Compound C-1-4

After dissolving 4-biphenyl boronic acid (14.3 g, 72 mmol), 1-iodo-3-bromobenzene (30.6 g, 108.3 mmol), Pd(PPh₃)₄ (3.3 g, 2.9 mmol), and Na₂CO₃ (22.9 g, 216 mmol) in a mixture solvent of toluene 500 mL and EtOH 120 mL; the mixture was stirred for 24 hours at 120° C. After the reaction, distilled water was slowly added to complete the reaction; an organic layer was extracted with ethylacetate (EA) from the mixture; the remaining moisture was removed using magnesium sulfate; dried; and then the remaining product was separated with a column to obtain compound C-1-4 (20 g, 90%).

### Preparation of Compound C-1-5

After dissolving compound C-1-4 (25 g, 80.8 mmol) in tetrahydrofuran (THF) 610 mL, the mixture was cooled to −78° C. After 10 minutes, n-BuLi (48.5 mL, 121.2 mmol) (2.5 M in hexane) was slowly added to the mixture, and stirred for 1 hour. Then, trimethylborate (18 mL, 161.7 mmol) was slowly added to the mixture, and stirred for 24 hours. After the reaction was completed, 1 M HCl was added to the mixture; extracted with ethylacetate; the remaining moisture was removed using magnesium sulfate; dried; and then the remaining product was recrystallized with methylchloride (MC)/hexane to obtain compound C-1-5 (16 g, 73%).

### Preparation of Compound C-1-6

After dissolving compound C-1-5 (16 g, 58.3 mmol), 2,4-dichloropyrimidine (11.3 g, 75.8 mmol), Pd(PPh₃)₄ (3.4 g, 2.91 mmol), and Na₂CO₃ (15.4 g, 146 mmol) in a mixture solvent of toluene 300 mL and EtOH 70 mL; the mixture was stirred for 24 hours at 120° C. After the reaction, distilled water was slowly added to complete the reaction; an organic layer was extracted with ethylacetate (EA) from the mixture; the remaining moisture was removed using magnesium sulfate; dried; and then the remaining product was separated with a column to obtain compound C-1-6 (10 g, 50%).

### Preparation of Compound C-25

After dissolving NaH (1.3 mg, 32.5 mmol) in dimethylformamide (DMF), the mixture was stirred. After dissolving compound C-1-3 (7 g, 21 mmol) in DMF; the mixture was added to the above reactant; and stirred for 1 hour. Then, after dissolving compound C-1-6 (8.6 g, 25.2 mmol) in DMF, and stirred; the above mixture which was stirred for 1 hour was added to the mixture; and then stirred for 24 hours at room temperature. After the reaction, the obtained solid was filtered; washed with ethylacetate; purified with column chromatography to obtain compound C-25 (5 g, 38%).

MS/FAB found 639; calculated 638.76

### EXAMPLE 2

### Preparation of Compound C-12

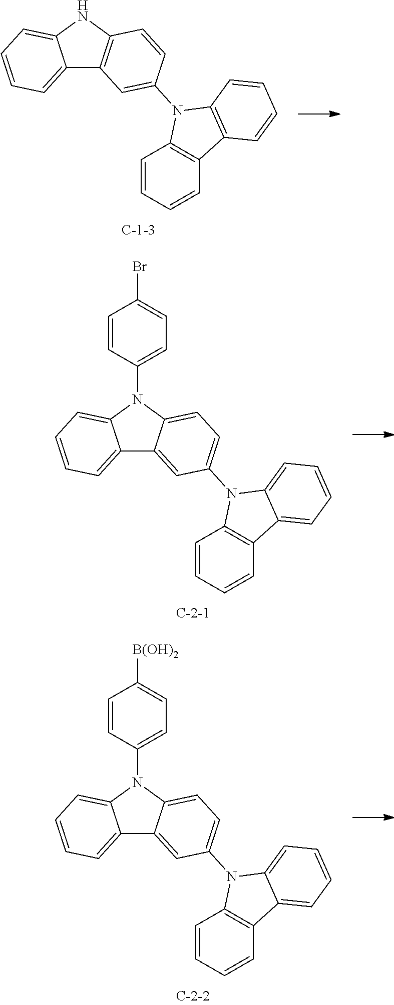 [SMILES: c1ccc2c(c1)[nH]c1ccc(-n3c4ccccc4c4ccccc43)cc12 ; Brc1ccc(-n2c3ccccc3c3cc(-n4c5ccccc5c5ccccc54)ccc32)cc1 ; OB(O)c1ccc(-n2c3ccccc3c3cc(-n4c5ccccc5c5ccccc54)ccc32)cc1]

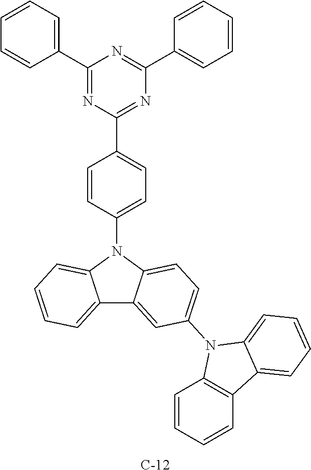

### Preparation of Compound C-2-1

After dissolving compound C-1-3 (10 g, 0.03 mol) 1-bromo-4-iodobenzene (17 g, 0.06 mol), CuI (3 g, 0.01 mol), and K₃PO₄ (16.5 g, 0.07 mmol) in toluene 160 mL, the mixture was stirred for 10 minutes at 80° C. Then, ethylenediamine (1 mL, 0.01 mol) was added to the mixture, and stirred for 12 hours at 140° C. After the reaction was completed, the mixture was extracted with ethylacetate; the organic layer was dried with MgSO₄; filtered; the solvent was removed under reduced pressure; and then the remaining product was separated with a column to obtain compound C-2-1 (13.6 g, 85%).

### Preparation of Compound C-2-2

While stirring the mixture of dry tetrahydrofuran (THF) 200 mL and compound C-2-1 (13.6 g, 0.028 mol) under nitrogen condition; n-BuLi (17 mL, solution of 2.25 M in hexane) was slowly added to the mixture at −78° C. Then, the mixture was stirred for 1 hour at −78° C.; B(O-iPr)₃ (13 mL, 0.06 mol) was slowly added to the mixture at −78° C.; and the mixture was heated to room temperature; and reacted for 12 hours. After the reaction was completed, the mixture was extracted with ethylacetate; the organic layer was dried with MgSO₄; filtered; the solvent was removed under reduced pressure; and then the remaining product was recrystallized to obtain compound C-2-2 (10.5 g, 83%).

### Preparation of Compound C-12

After dissolving compound C-2-2 (10.5 g, 0.02 mol), 2-chloro-4,6-diphenyl-1,3,5-triazine (7.5 g, 0.03 mol), Pd(PPh₃)₄ (1.34 g, 0.001 mol), and K₂CO₃ (9.63 g, 0.07 mol) in a mixture solvent of toluene 116 mL, EtOH 30 mL, and H₂O 35 mL; the mixture was stirred for 12 hours at 60° C. After the reaction was completed, the mixture was extracted with ethylacetate; the organic layer was dried with MgSO₄; filtered; the solvent was removed under reduced pressure; and then the remaining product was recrystallized to obtain compound C-12 (6.2 g, 42%).

MS/FAB found 640; calculated 639.75

### EXAMPLE 3

### Preparation of Compound C-14

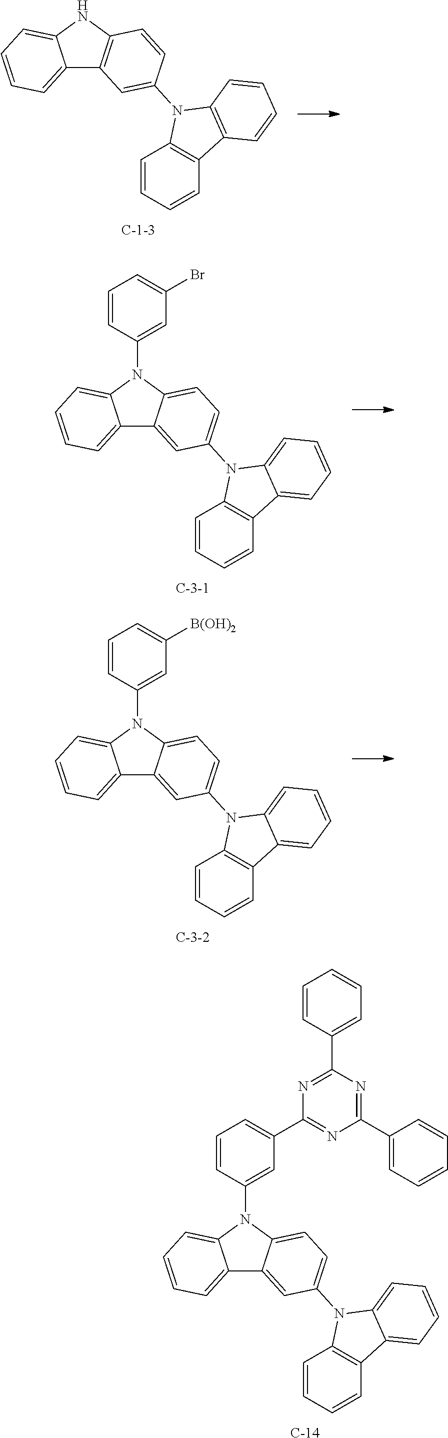

### Preparation of Compound C-3-1

After dissolving compound C-1-3 (10 g, 0.03 mol) 1,3-dibromobenzene (120 g, 0.09 mol), CuI (3 g, 0.5 mol), and K₃PO₄ (16.5 g, 0.07 mmol) in toluene 160 mL; the mixture was stirred for 10 minutes at 80° C. Then, ethylenediamine (1 mL, 0.01 mol) was added to the mixture, and stirred for 12 hours at 140° C. After the reaction was completed, the mixture was extracted with ethylacetate; the organic layer was dried with MgSO₄; filtered; the solvent was removed under reduced pressure; and then the remaining product was separated with a column to obtain compound C-3-1 (10.2 g, 68%).

### Preparation of Compound C-3-2

While stirring the mixture of THF 160 mL and compound C-3-1 (10.2 g, 0.021 mol) under nitrogen condition, n-BuLi (13 mL, solution of 2.25 M in hexane) was slowly added to the mixture at −78° C. Then, the mixture was stirred for 1 hour at −78° C.; B(O-iPr)₃ (9.6 mL, 0.04 mol) was slowly added to the mixture at −78° C.; and the mixture was heated to room temperature; and reacted for 12 hours. After the reaction was completed, the mixture was extracted with ethylacetate; the organic layer was dried with MgSO₄; filtered; the solvent was removed under reduced pressure; and then the remaining product was recrystallized to obtain compound C-3-2 (7.3 g, 77%).

### Preparation of Compound C-14

After dissolving compound C-3-2 (7.3 g, 0.02 mol), 2-chloro-4,6-diphenyl-1,3,5-triazine (5.2 g, 0.02 mol), Pd(PPh₃)₄ (0.93 g, 0.0008 mol), and K₂CO₃ (6.7 g, 0.04 mol) in a mixture solvent of toluene 80 mL, EtOH 20 mL, and distilled water 25 mL; the mixture was stirred for 12 hours at 60° C. After the reaction was completed, the mixture was extracted with ethylacetate; the organic layer was dried with MgSO₄; filtered; the solvent was removed under reduced pressure; and then the remaining product was recrystallized to obtain compound C-14 (3.9 g, 38%).

MS/FAB found 640; calculated 639.75

### EXAMPLE 4

### Preparation of Compound C-53

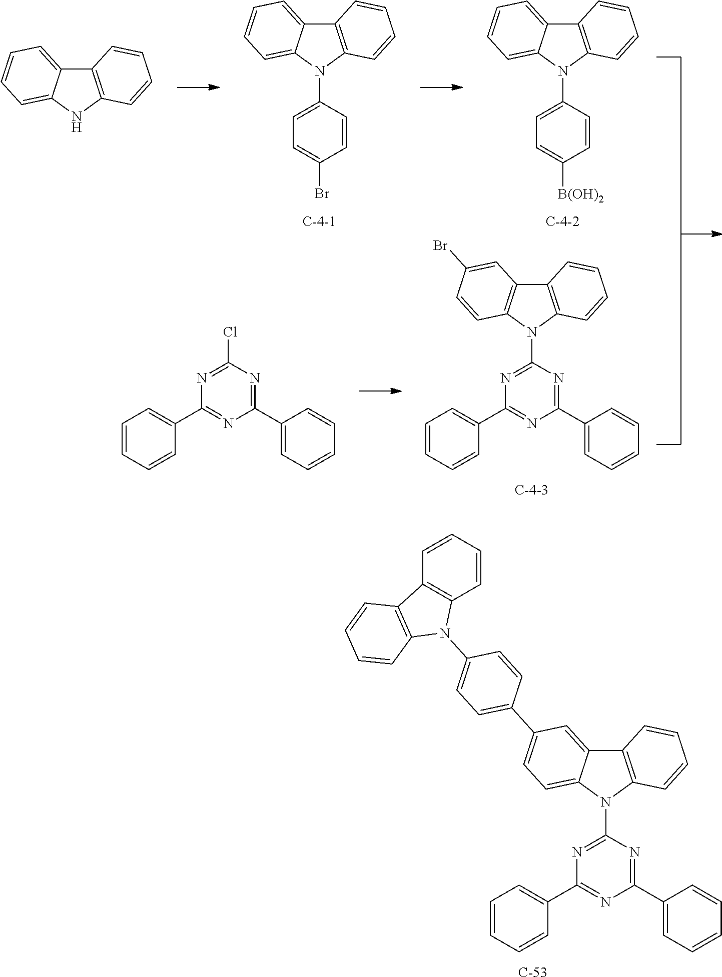

### Preparation of Compound C-4-1

After mixing 9H-carbazole (20 g, 119.6 mmol), 1-bromo-4-iodobenzene (68 g, 240.3 mmol), CuI (11.4 g, 59.8 mmol), ethylenediamine (8 mL, 119.6 mmol), K₃PO₄ (50.88 g, 240 mmol), and toluene 200 mL in a 500 mL round-bottom flask; the mixture was stirred for 5 hours under reflux. After the reaction was completed, the mixture was cooled to room temperature; extracted with dichloromethane (DCM) and H₂O; and the DCM layer was dried with MgSO₄. Then, the DCM layer was concentrated under reduced pressure, and filtered through silica gel with a column. Then, the obtained solvent was concentrated under reduced pressure to obtain compound C-4-1 (33.8 g, 85%).

### Preparation of Compound C-4-2

After mixing compound C-4-1 (10 g, 31.0 mmol), and THF 150 mL in a 500 mL round-bottom flask; the mixture was cooled to −78° C. Then, 2.5 M n-butyl lithium (14.8 mL, 37.2 mmol) was added to the mixture, and after 1 hour, isopropyl borate (10.73 mL, 46.5 mmol) was added to the mixture. After 19 hours, the mixture was extracted with EA and H₂O, and the EA layer was dried with MgSO₄. Then, the EA layer was concentrated to obtain compound C-4-2 (6.42 g, 72%).

### Preparation of Compound C-4-3

After mixing 3-bromocarbazole (10 g, 40.63 mmol), and DMF 300 mL in a 500 mL round-bottom flask; the mixture was cooled to 0° C. Then, NaH was added to the mixture, and stirred for 10 minutes. Then, 2-chloro-4,6-diphenyl-1,3,5-triazine (13.05 g, 48.76 mmol) was added and the mixture was reacted at room temperature. After 17 hours, the mixture was quenched with MeOH, and filtered to obtain compound C-4-3 (18.75 g, 96%).

### Preparation of Compound C-53

After mixing compound C-4-2 (3.5 g, 12.19 mmol), compound C-4-3 (7 g, 14.63 mmol), Pd(PPh₃)₄ (422.0 mg, 0.36 mmol), K₂CO₃ (3.36 g, 24.38 mol), toluene 80 mL, EtOH 20 mL, and H₂O 20 mL in a 500 mL round-bottom flask; the mixture was stirred under reflux. After 15 hours, an obtained white solid was filtered through silica gel with a column to obtain compound C-53 (1.5 g, 19%).

MS/FAB found 640; calculated 639.75

### EXAMPLE 5

### Preparation of Compound C-54

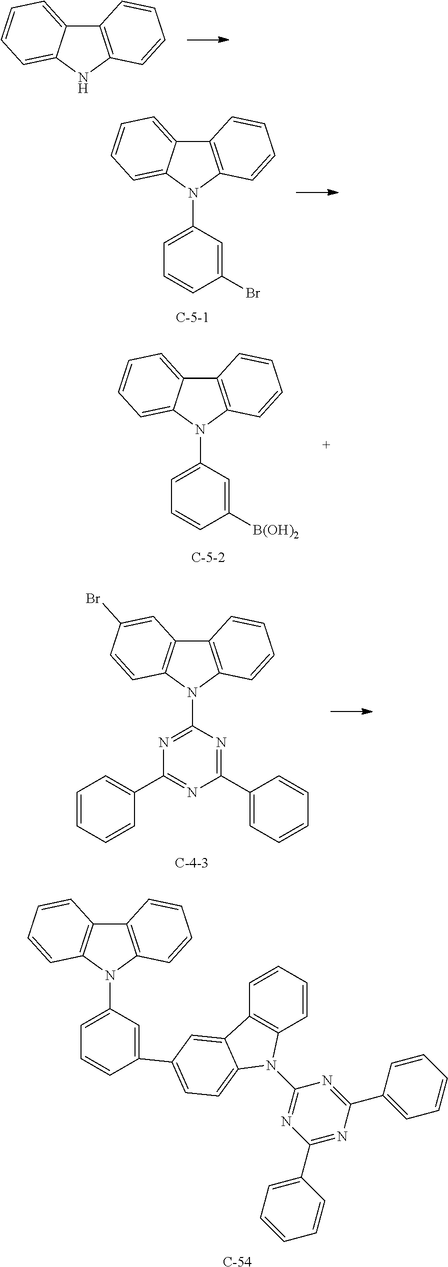

### Preparation of Compound C-5-1

After mixing 9H-carbazole (60 g, 350.8 mmol), 1-bromo-3-iodobenzene (202 g, 717.6 mmol), CuI (33.4 g, 175.4 mmol), ethylenediamine (23 mL, 350.8 mmol), K₃PO₄ (152.1 g, 717.6 mmol), and toluene 400 mL in a 1 L round-bottom flask, the mixture was stirred for 23 hours under reflux. After the reaction was completed, the mixture was cooled to room temperature; extracted with DCM and H₂O; and the DCM layer was dried with MgSO₄. Then, the DCM layer was concentrated under reduced pressure, and filtered through silica gel with a column. Then, the obtained solvent was concentrated under reduced pressure to obtain compound C-5-1 (68 g, 61%).

### Preparation of Compound C-5-2

After mixing compound C-5-1 (10 g, 31.0 mmol), and THF 150 mL in a 500 mL round-bottom flask, the mixture was cooled to −78° C. Then, 2.5 M n-butyl lithium (14.8 mL, 37.2 mmol) was added to the mixture, and after 1 hour, isopropyl borate (10.73 mL, 46.5 mmol) was added to the mixture. After 18 hours, the mixture was extracted with EA and H₂O, and the EA layer was dried with MgSO₄. Then, the EA layer was concentrated to obtain compound C-5-2 (6.42 g, 68%).

### Preparation of Compound C-54

After mixing compound C-5-2 (3.0 g, 10.4 mmol), compound C-4-3 (6 g, 12.5 mmol), Pd(PPh₃)₄ (362 mg, 0.31 mmol), K₂CO₃ (2.88 g, 20.89 mol), toluene 80 mL, EtOH 20 mL, and H₂O 20 mL in a 500 mL round-bottom flask; the mixture was stirred under reflux. After 17 hours, completing the reaction, the mixture was extracted with DCM and H₂O, and the DCM layer was concentrated under reduced pressure. Then, the concentrated DCM layer was filtered through silica gel with a column to obtain compound C-54 (1.2 g, 18%).

MS/FAB found 640; calculated 639.75

### Device Example 1

### Production of an OLED Device Using the Compound According to the Present Invention

An OLED device was produced using the compound according to the present invention. A transparent electrode indium tin oxide (ITO) thin film (15 Ω/sq) on a glass substrate for an organic light-emitting diode (OLED) device (Samsung Corning, Republic of Korea) was subjected to an ultrasonic washing with trichloroethylene, acetone, ethanol and distilled water, sequentially, and then was stored in isopropanol. Then, the ITO substrate was mounted on a substrate holder of a vacuum vapor depositing apparatus. N¹,N^{1′}-([1,1′-biphenyl]-4,4′-diyl)bis(N¹-(naphthalen-1-yl)-N⁴,N⁴-diphenylbenzen-1,4-diamine) was introduced into a cell of said vacuum vapor depositing apparatus, and then the pressure in the chamber of said apparatus was controlled to 10⁻⁶ torr. Thereafter, an electric current was applied to the cell to evaporate the above introduced material, thereby forming a hole injection layer having a thickness of 60 nm on the ITO substrate. Then, N,N′-di(4-biphenyl)-N,N′-di(4-biphenyl)-4,4′-diaminobiphenyl was introduced into another cell of said vacuum vapor depositing apparatus, and was evaporated by applying an electric current to the cell, thereby forming a hole transport layer having a thickness of 20 nm on the hole injection layer. Thereafter, compound C-25 was introduced into one cell of the vacuum vapor depositing apparatus, as a host material, and compound D-1 was introduced into another cell as a dopant. The two materials were evaporated at different rates and were deposited in a doping amount of 15 wt % based on the total amount of the host and dopant to form a light-emitting layer having a thickness of 30 nm on the hole transport layer. Then, 2-(4-(9,10-di(naphthalen-2-yl)anthracen-2-yl)phenyl)-1-phenyl-1H-benzo[d]imidazole was introduced into one cell and lithium quinolate was introduced into another cell. The two materials were evaporated at the same rate and were deposited in a doping amount of 50 wt % each to form an electron transport layer having a thickness of 30 nm on the light-emitting layer. Then, after depositing lithium quinolate as an electron injection layer having a thickness of 2 nm on the electron transport layer, an Al cathode having a thickness of 150 nm was deposited by another vacuum vapor deposition apparatus on the electron injection layer. Thus, an OLED device was produced. All the materials used for producing the OLED device were purified by vacuum sublimation at 10⁻⁶ torr prior to use.

The produced OLED device showed a green emission having a luminance of 1060 cd/m² and a current density of 2.00 mA/cm² at a driving voltage of 3.8 V.

### Device Example 2

### Production of an OLED Device Using the Compound According to the Present Invention

An OLED device was produced in the same manner as in Device Example 1, except for using compound C-12 as a host, and using compound D-9 as a dopant of the light emitting material.

The produced OLED device showed a green emission having a luminance of 1050 cd/m² and a current density of 2.88 mA/cm² at a driving voltage of 3.1 V.

### Device Example 3

### Production of an OLED Device Using the Compound According to the Present Invention

An OLED device was produced in the same manner as in Device Example 1, except for using compound C-14 as a host, and using compound D-9 as a dopant of the light emitting material.

The produced OLED device showed a green emission having a luminance of 1040 cd/m² and a current density of 2.63 mA/cm² at a driving voltage of 3.2 V.

### Comparative Example 1

### Production of an OLED Device Using Conventional Electroluminescent Compounds

An OLED device was produced in the same manner as in Device Example 1, except for depositing the light emitting layer using compound D-5 as a host of the light emitting material, and compound Ir(ppy)₃ [tris(2-phenylpyridine)iridium] as a dopant in another cell; and depositing aluminum(III)bis(2-methyl-8-quinolinato)4-phenylphenolate to form a hole blocking layer having a thickness of 10 nm on the light emitting layer.

The produced OLED device showed a green emission having a luminance of 1000 cd/m² and a current density of 2.86 mA/cm² at a driving voltage of 4.9 V.

It is verified that the organic electroluminescent compounds of the present invention have superior luminous efficiency over conventional host compounds. Notably, the devices using the compounds according to the present invention as a luminescent host material have superior luminous characteristics. In addition, the compounds can improve the power efficiency of the device by decreasing the driving voltage, to reduce overall power consumption.

1. An organic electroluminescent compound represented by the following formula 1: 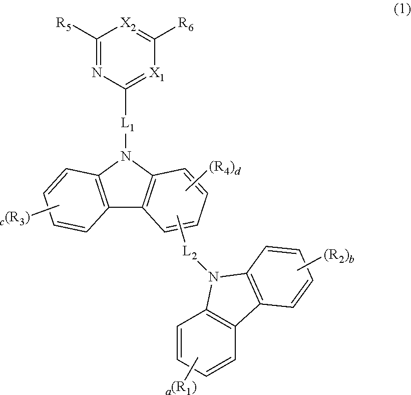
wherein
L₁ and L₂ each independently represent a single bond, a substituted or unsubstituted 5- to 30-membered heteroarylene, or a substituted or unsubstituted (C6-C30)arylene;
X₁ and X₂ each independently represent CR₇ or N;
R₁ to R₄ and R₇ each independently represent hydrogen, deuterium, a halogen, a cyano, a carboxyl, a nitro, a hydroxyl, a substituted or unsubstituted (C1-C30)alkyl, a substituted or unsubstituted (C2-C30)alkenyl, a substituted or unsubstituted (C2-C30)alkynyl, a substituted or unsubstituted (C1-C30)alkoxy, a substituted or unsubstituted (C3-C30)cycloalkyl, a substituted or unsubstituted (C3-C30)cycloalkenyl, a substituted or unsubstituted 3- to 7-membered heterocycloalkyl, a substituted or unsubstituted (C6-C30)aryl, a substituted or unsubstituted 5- to 30-membered heteroaryl, —NR₁₁R₁₂, —SiR₁₃R₁₄R₁₅, —SR₁₆, —OR₁₇, —COR₁₈ or —B(OR₁₉)(OR₂₀); or are linked to an adjacent substituent(s) to form a mono- or polycyclic, 3- to 30-membered alicyclic or aromatic ring whose carbon atom(s) may be replaced with at least one hetero atom selected from the group consisting of nitrogen, oxygen and sulfur;
R₅ and R₆ each independently represent hydrogen, deuterium, a halogen, a substituted or unsubstituted (C1-C30)alkyl, a substituted or unsubstituted (C6-C30)aryl, a substituted or unsubstituted 5- to 30-membered heteroaryl, —NR₁₁R₁₂ or —SiR₁₃R₁₄R₁₅;
R₁₁ to R₂₀ each independently represent hydrogen, deuterium, a halogen, a cyano, a carboxyl, a nitro, a hydroxyl, a substituted or unsubstituted (C1-C30)alkyl, a substituted or unsubstituted (C2-C30)alkenyl, a substituted or unsubstituted (C2-C30)alkynyl, a substituted or unsubstituted (C1-C30)alkoxy, a substituted or unsubstituted (C3-C30)cycloalkyl, a substituted or unsubstituted (C3-C30)cycloalkenyl, a substituted or unsubstituted 3- to 7-membered heterocycloalkyl, a substituted or unsubstituted (C6-C30)aryl, or a substituted or unsubstituted 5- to 30-membered heteroaryl; or are linked to an adjacent substituent(s) to form a mono- or polycyclic, 3- to 30-membered alicyclic or aromatic ring whose carbon atom(s) may be replaced with at least one hetero atom selected from the group consisting of nitrogen, oxygen and sulfur;
a, b and c each independently represent an integer of 1 to 4; where a, b or c is an integer of 2 or more, each of R₁, each of R₂, or each of R₃ may be same or different;
d represents an integer of 1 to 3; where d is an integer of 2 or more, each of R₄ may be same or different;
the heteroarylene and heteroaryl contain at least one hetero atom selected from B, N, O, S, P(═O), Si and P; and
the heterocycloalkyl contains at least one hetero atom selected from O, S and N.

2. The organic electroluminescent compound according to claim 1, wherein the compound represented by formula 1 is represented by one selected from formulae 2 to 4: 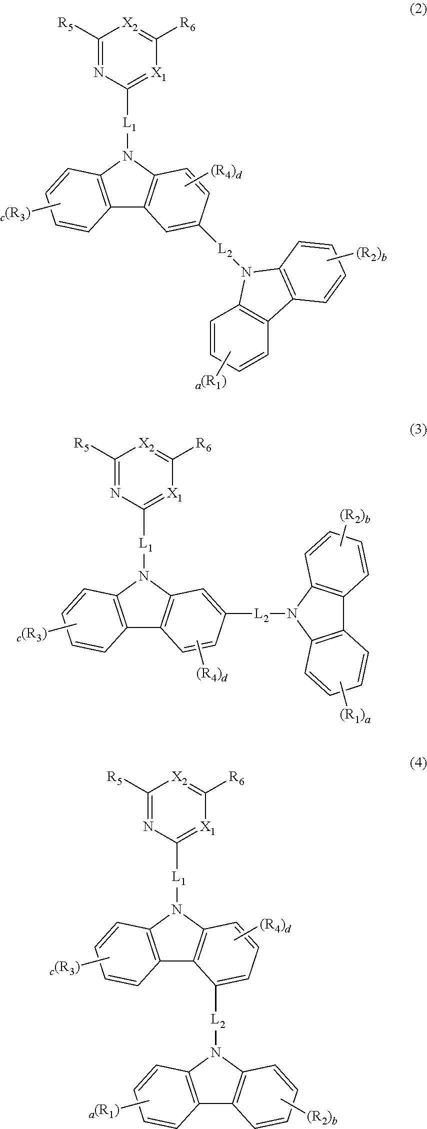
wherein L₁, L₂, X₁, X₂, R₁ to R₆, a, b, c and d are as defined in claim 1.

3. The organic electroluminescent compound according to claim 1, wherein the substituents of the substituted (C1-C30)alkyl, the substituted (C2-C30)alkenyl, the substituted (C2-C30)alkynyl, the substituted (C1-C30)alkoxy, the substituted (C3-C30)cycloalkyl, the substituted (C3-C30)cycloalkenyl, the substituted 3- to 7-membered heterocycloalkyl, the substituted (C6-C30)aryl(ene), and the substituted 5- to 30-membered heteroaryl(ene) in L₁, L₂, R₁ to R₇, R₁₁ to R₂₀ each independently are at least one selected from the group consisting of deuterium; a halogen; a cyano; a carboxyl; a nitro; a hydroxyl; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a 3- to 7-membered heterocycloalkyl; a (C6-C30)aryloxy; a (C6-C30)arylthio; a 5- to 30-membered heteroaryl unsubstituted or substituted with a (C6-C30)aryl; a (C6-C30)aryl unsubstituted or substituted with a 5- to 30-membered heteroaryl; a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di-(C1-C30)alkylamino; a mono- or di-(C6-C30)arylamino; a (C1-C30)alkyl(C6-C30)arylamino; a (C1-C30)alkylcarbonyl; a (C1-C30)alkoxycarbonyl; a (C6-C30)arylcarbonyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl.

4. The organic electroluminescent compound according to claim 1, wherein in formula 1,
L₁ and L₂ each independently represent a single bond, a substituted or unsubstituted 5- to 15-membered heteroarylene, or a substituted or unsubstituted (C6-C20)arylene;
X₁ and X₂ each independently represent CR₇ or N; where R₇ represents hydrogen, a substituted or unsubstituted (C6-C30)aryl, or a substituted or unsubstituted 5- to 15-membered heteroaryl;
R₁ to R₄ each independently represent hydrogen, a halogen, a substituted or unsubstituted (C6-C30)aryl, a substituted or unsubstituted 5- to 30-membered heteroaryl, —NR₁₁R₁₂ or —SiR₁₃R₁₄R₁₅; or are linked to an adjacent substituent(s) to form a mono- or polycyclic, 3- to 30-membered alicyclic or aromatic ring; where R₁₁ and R₁₂ each independently represent a substituted or unsubstituted (C6-C30)aryl, or a substituted or unsubstituted 5- to 30-membered heteroaryl, and R₁₃ to R₁₅ each independently represent a substituted or unsubstituted (C1-C30)alkyl, or a substituted or unsubstituted (C6-C30)aryl;
R₅ and R₆ each independently represent hydrogen, a substituted or unsubstituted (C6-C30)aryl, a substituted or unsubstituted 5- to 30-membered heteroaryl, or —SiR₁₃R₁₄R₁₅; where R₁₃ to R₁₅ each independently represent a substituted or unsubstituted (C1-C30)alkyl, or a substituted or unsubstituted (C6-C30)aryl.

5. The organic electroluminescent compound according to claim 1, wherein in formula 1,
L₁ and L₂ each independently represent a single bond; a 5- to 15-membered heteroarylene unsubstituted or substituted with a (C1-C6)alkyl or a (C6-C15)aryl; or a (C6-C20)arylene unsubstituted or substituted with a (C1-C6)alkyl or a (C6-C15)aryl;
X₁ and X₂ each independently represent CR₇ or N; where R₇ represents hydrogen; a 5- to 15-membered heteroaryl unsubstituted or substituted with a (C1-C6)alkyl or a (C6-C15)aryl; or a (C6-C20)aryl unsubstituted or substituted with a (C1-C6)alkyl or a (C6-C15)aryl;
R₁ to R₄ each independently represent hydrogen; a halogen; a (C6-C20)aryl unsubstituted or substituted with a (C1-C6)alkyl; a 5- to 15-membered heteroaryl unsubstituted or substituted with a (C1-C6)alkyl; —NR₁₁R₁₂; or —SiR₁₃R₁₄R₁₅, or are linked to an adjacent substituent(s) to form a mono- or polycyclic, 3- to 15-membered aromatic ring; where R₁₁ and R₁₂ each independently represent an unsubstituted (C6-C20)aryl, or an unsubstituted 5- to 15-membered heteroaryl, and R₁₃ to R₁₅ each independently represent an unsubstituted (C1-C10)alkyl, or an unsubstituted (C6-C15)aryl;
R₅ and R₆ each independently represent hydrogen; a (C6-C20)aryl unsubstituted or substituted with deuterium, a halogen, a (C1-C6)alkyl, a (C6-C20)aryl, a tri(C6-C15)arylsilyl, a di(C6-C15)arylamino, a (C1-C6)alkyl(C6-C20)aryl, a (C1-C6)alkyldi(C6-C15)arylsilyl or a 5- to 15-membered heteroaryl; a 5- to 15-membered heteroaryl unsubstituted or substituted with a (C1-C6)alkyl or a (C6-C12)aryl; or —SiR₁₃R₁₄R₁₅, where R₁₃ to R₁₅ each independently represent an unsubstituted (C1-C10)alkyl, or an unsubstituted (C6-C15)aryl.

6. The organic electroluminescent compound according to claim 1, wherein the compound represented by formula 1 is selected from the group consisting of:

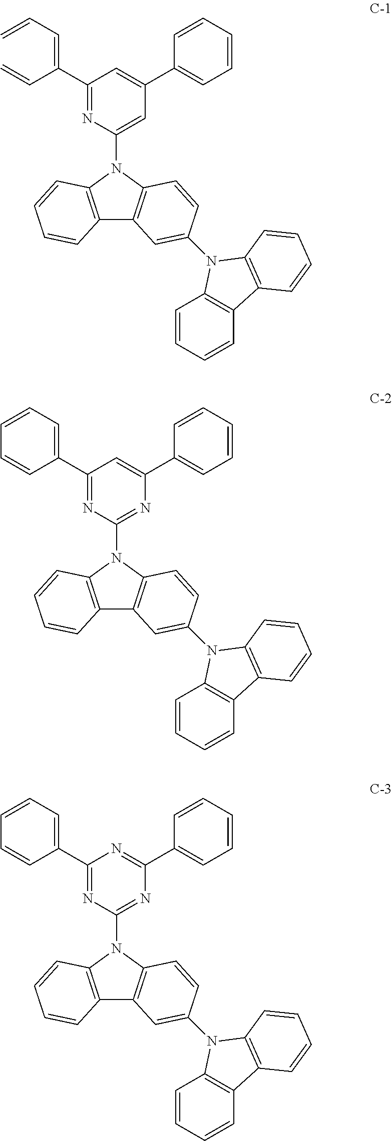 [SMILES: C=C/C=C(\C=C)c1cc(-c2ccccc2)cc(-n2c3ccccc3c3cc(-n4c5ccccc5c5ccccc54)ccc32)n1 ; c1ccc(-c2cc(-c3ccccc3)nc(-n3c4ccccc4c4cc(-n5c6ccccc6c6ccccc65)ccc43)n2)cc1 ; c1ccc(-c2nc(-c3ccccc3)nc(-n3c4ccccc4c4cc(-n5c6ccccc6c6ccccc65)ccc43)n2)cc1]

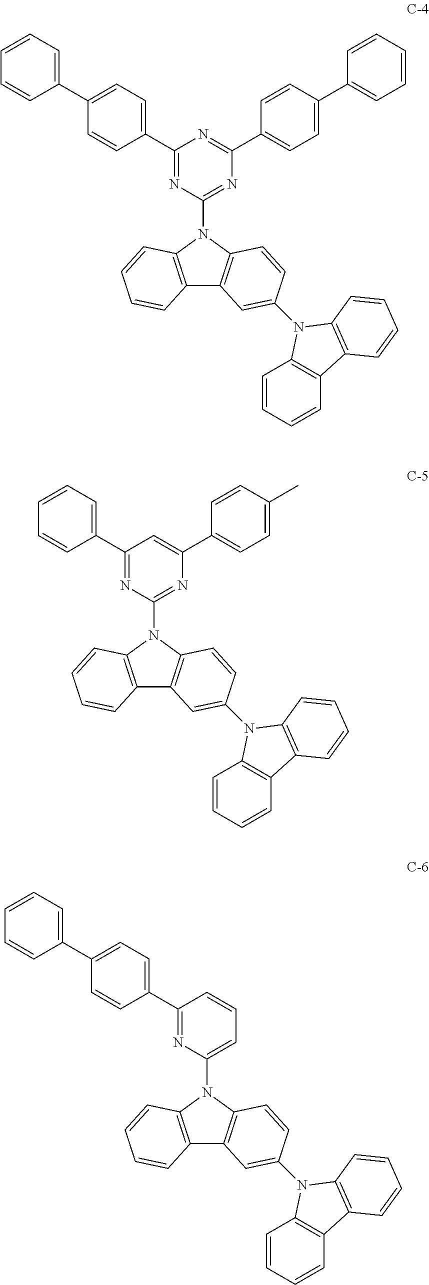 [SMILES: c1ccc(-c2ccc(-c3nc(-c4ccc(-c5ccccc5)cc4)nc(-n4c5ccccc5c5cc(-n6c7ccccc7c7ccccc76)ccc54)n3)cc2)cc1 ; Cc1ccc(-c2cc(-c3ccccc3)nc(-n3c4ccccc4c4cc(-n5c6ccccc6c6ccccc65)ccc43)n2)cc1 ; c1ccc(-c2ccc(-c3cccc(-n4c5ccccc5c5cc(-n6c7ccccc7c7ccccc76)ccc54)n3)cc2)cc1]

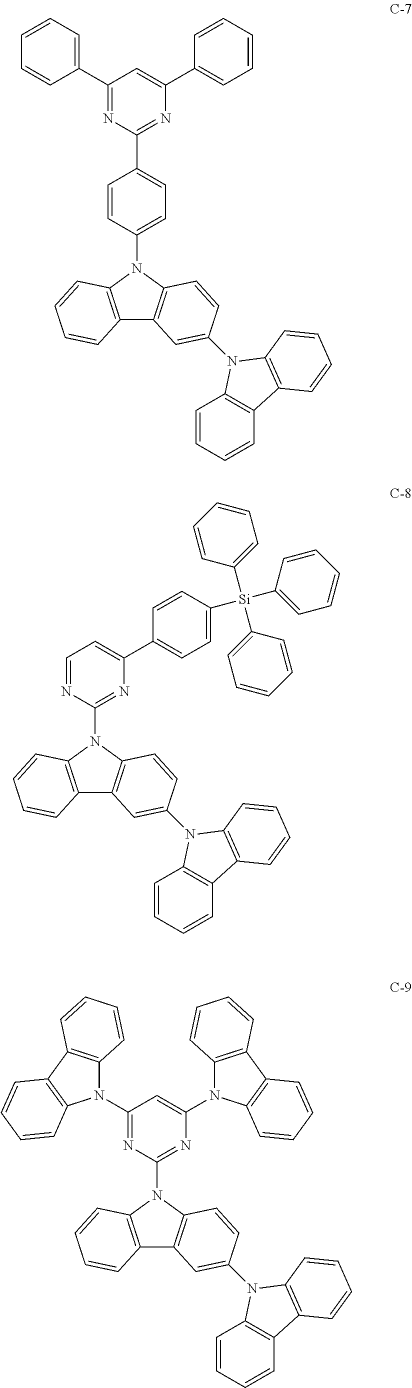

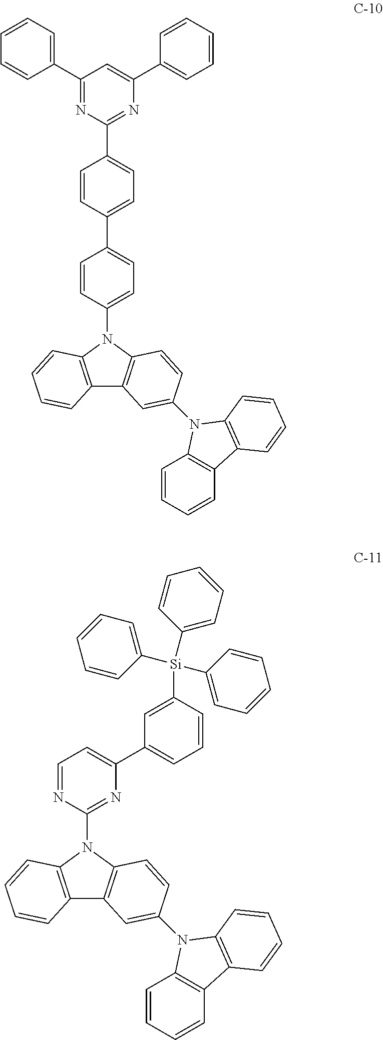 [SMILES: c1ccc(-c2cc(-c3ccccc3)nc(-c3ccc(-c4ccc(-n5c6ccccc6c6cc(-n7c8ccccc8c8ccccc87)ccc65)cc4)cc3)n2)cc1 ; c1ccc([Si](c2ccccc2)(c2ccccc2)c2cccc(-c3ccnc(-n4c5ccccc5c5cc(-n6c7ccccc7c7ccccc76)ccc54)n3)c2)cc1]

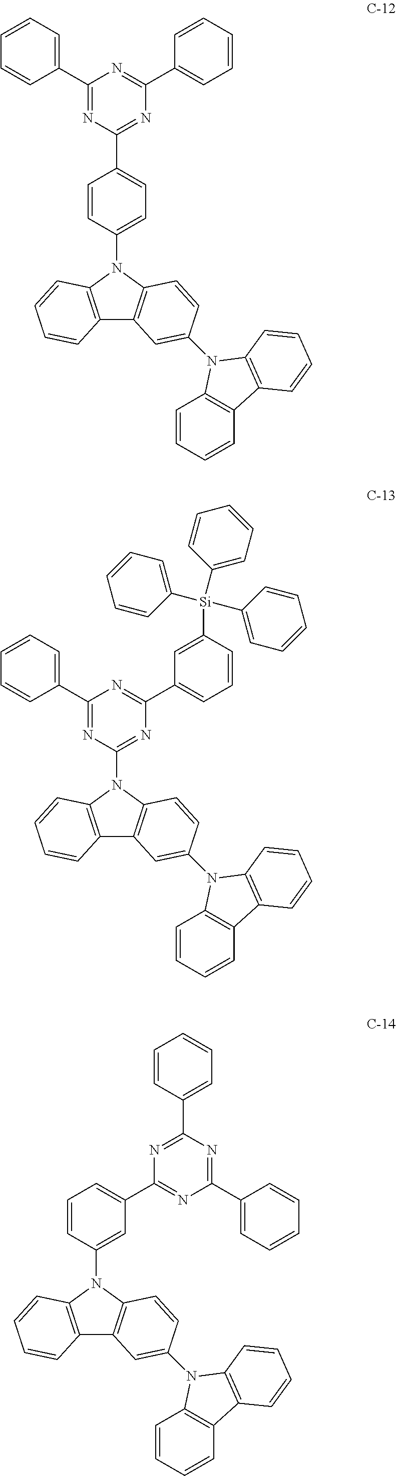 [SMILES: c1ccc(-c2nc(-c3ccccc3)nc(-c3ccc(-n4c5ccccc5c5cc(-n6c7ccccc7c7ccccc76)ccc54)cc3)n2)cc1 ; c1ccc(-c2nc(-c3cccc([Si](c4ccccc4)(c4ccccc4)c4ccccc4)c3)nc(-n3c4ccccc4c4cc(-n5c6ccccc6c6ccccc65)ccc43)n2)cc1 ; c1ccc(-c2nc(-c3ccccc3)nc(-c3cccc(-n4c5ccccc5c5cc(-n6c7ccccc7c7ccccc76)ccc54)c3)n2)cc1]

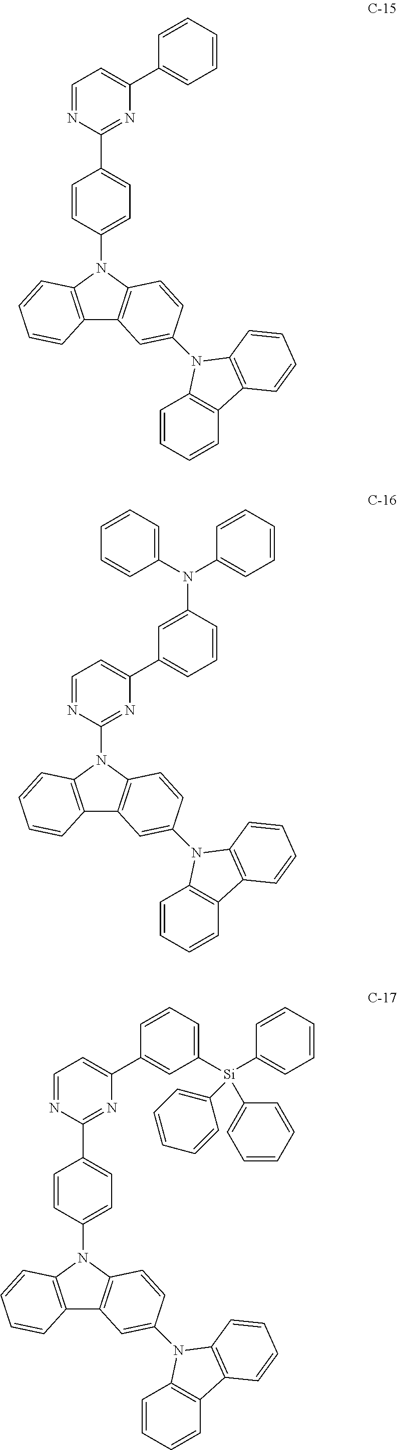 [SMILES: c1ccc(-c2ccnc(-c3ccc(-n4c5ccccc5c5cc(-n6c7ccccc7c7ccccc76)ccc54)cc3)n2)cc1 ; c1ccc(N(c2ccccc2)c2cccc(-c3ccnc(-n4c5ccccc5c5cc(-n6c7ccccc7c7ccccc76)ccc54)n3)c2)cc1 ; c1ccc([Si](c2ccccc2)(c2ccccc2)c2cccc(-c3ccnc(-c4ccc(-n5c6ccccc6c6cc(-n7c8ccccc8c8ccccc87)ccc65)cc4)n3)c2)cc1]

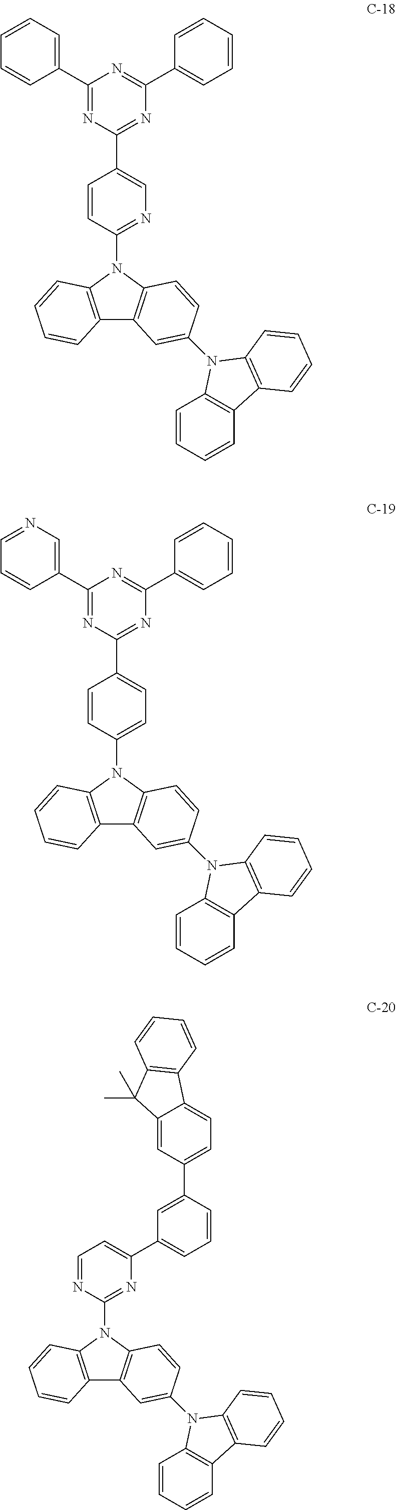 [SMILES: c1ccc(-c2nc(-c3ccccc3)nc(-c3ccc(-n4c5ccccc5c5cc(-n6c7ccccc7c7ccccc76)ccc54)nc3)n2)cc1 ; c1ccc(-c2nc(-c3ccc(-n4c5ccccc5c5cc(-n6c7ccccc7c7ccccc76)ccc54)cc3)nc(-c3cccnc3)n2)cc1 ; CC1(C)c2ccccc2-c2ccc(-c3cccc(-c4ccnc(-n5c6ccccc6c6cc(-n7c8ccccc8c8ccccc87)ccc65)n4)c3)cc21]

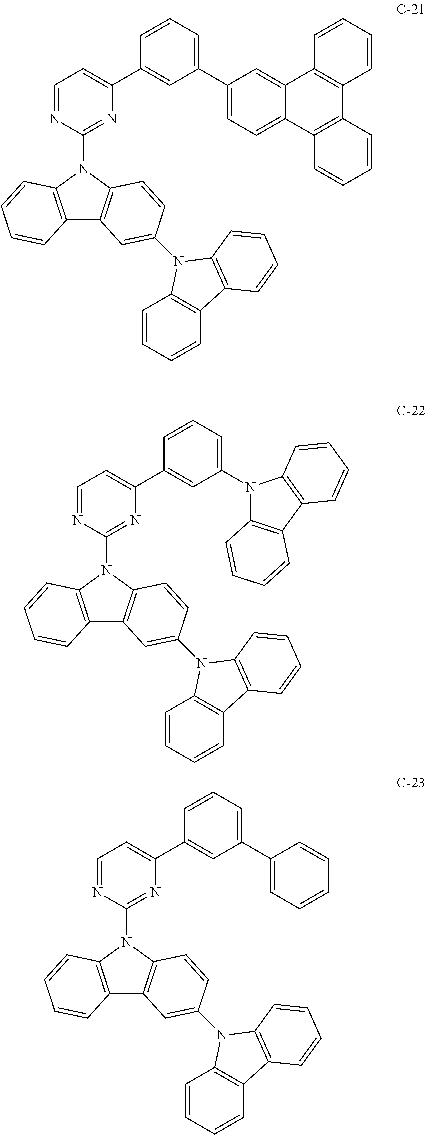

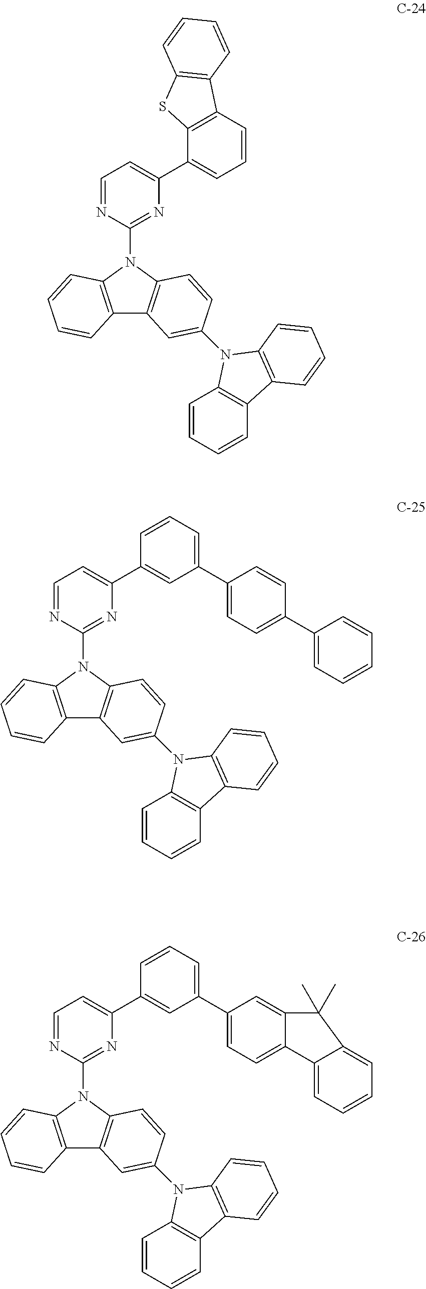 [SMILES: c1ccc2c(c1)sc1c(-c3ccnc(-n4c5ccccc5c5cc(-n6c7ccccc7c7ccccc76)ccc54)n3)cccc12 ; c1ccc(-c2ccc(-c3cccc(-c4ccnc(-n5c6ccccc6c6cc(-n7c8ccccc8c8ccccc87)ccc65)n4)c3)cc2)cc1 ; CC1(C)c2ccccc2-c2ccc(-c3cccc(-c4ccnc(-n5c6ccccc6c6cc(-n7c8ccccc8c8ccccc87)ccc65)n4)c3)cc21]

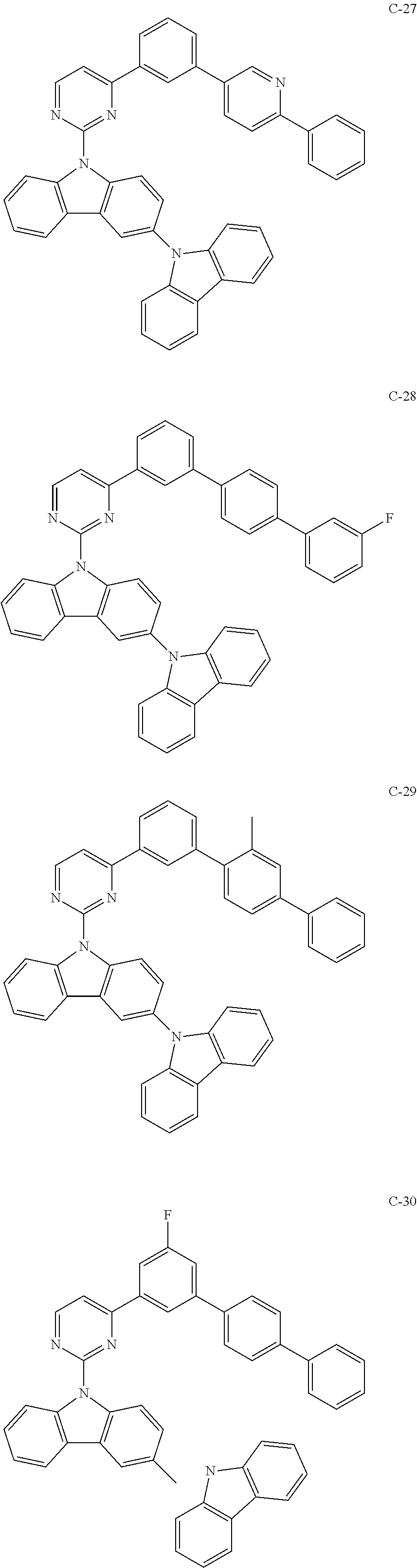

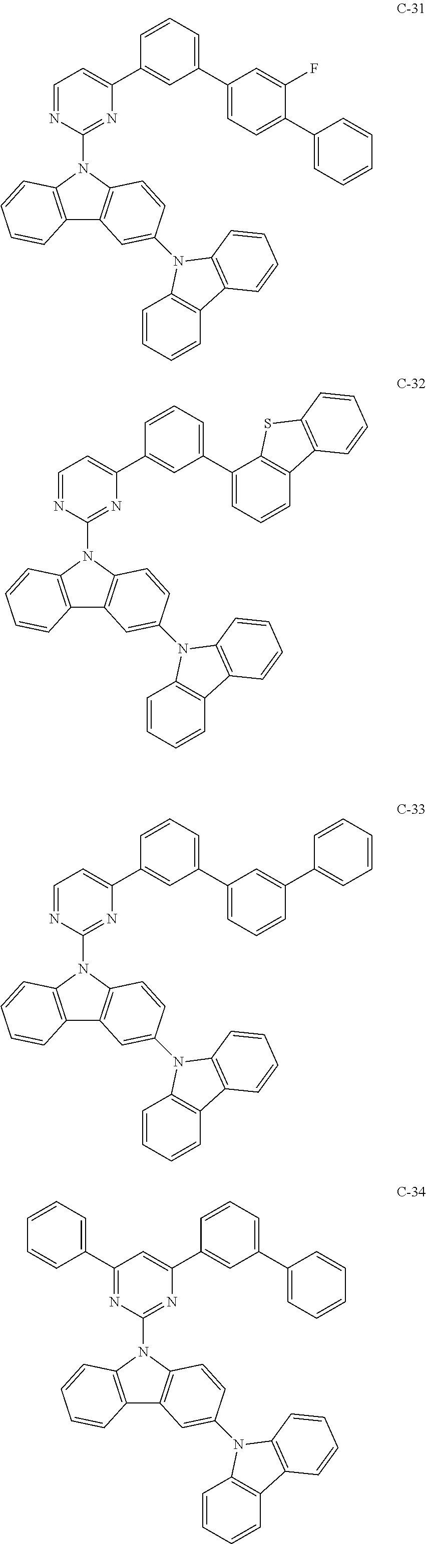

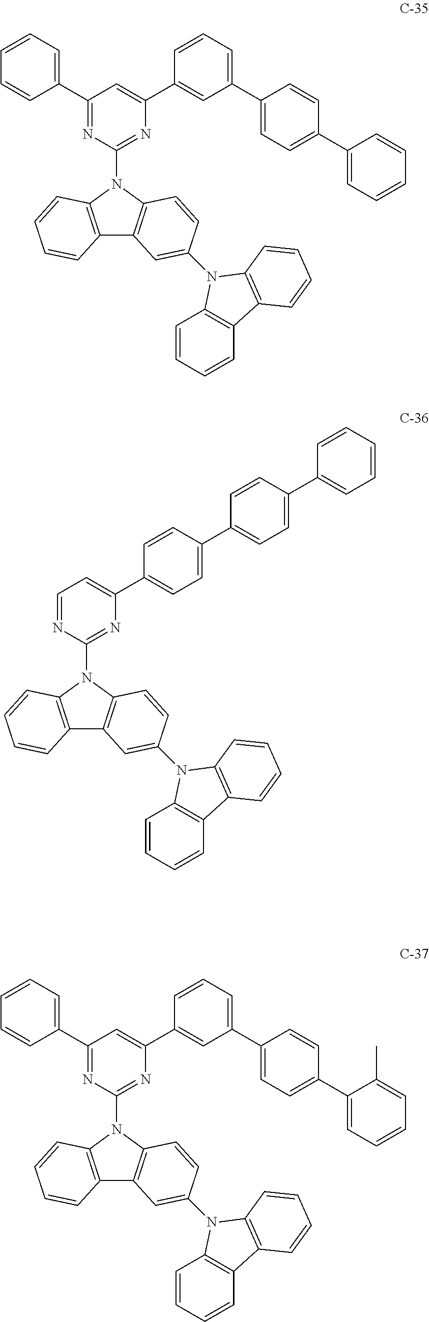 [SMILES: c1ccc(-c2ccc(-c3cccc(-c4cc(-c5ccccc5)nc(-n5c6ccccc6c6cc(-n7c8ccccc8c8ccccc87)ccc65)n4)c3)cc2)cc1 ; c1ccc(-c2ccc(-c3ccc(-c4ccnc(-n5c6ccccc6c6cc(-n7c8ccccc8c8ccccc87)ccc65)n4)cc3)cc2)cc1 ; Cc1ccccc1-c1ccc(-c2cccc(-c3cc(-c4ccccc4)nc(-n4c5ccccc5c5cc(-n6c7ccccc7c7ccccc76)ccc54)n3)c2)cc1]

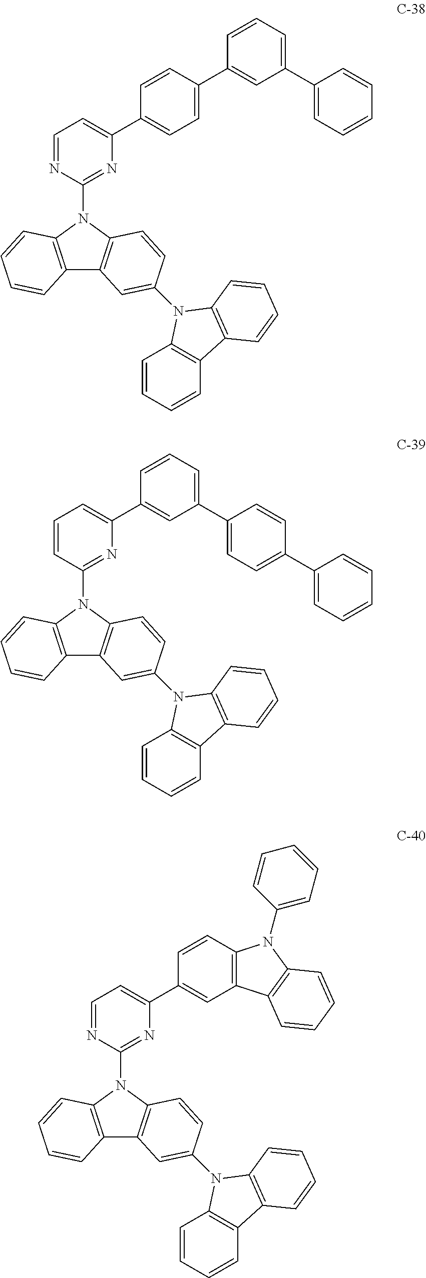

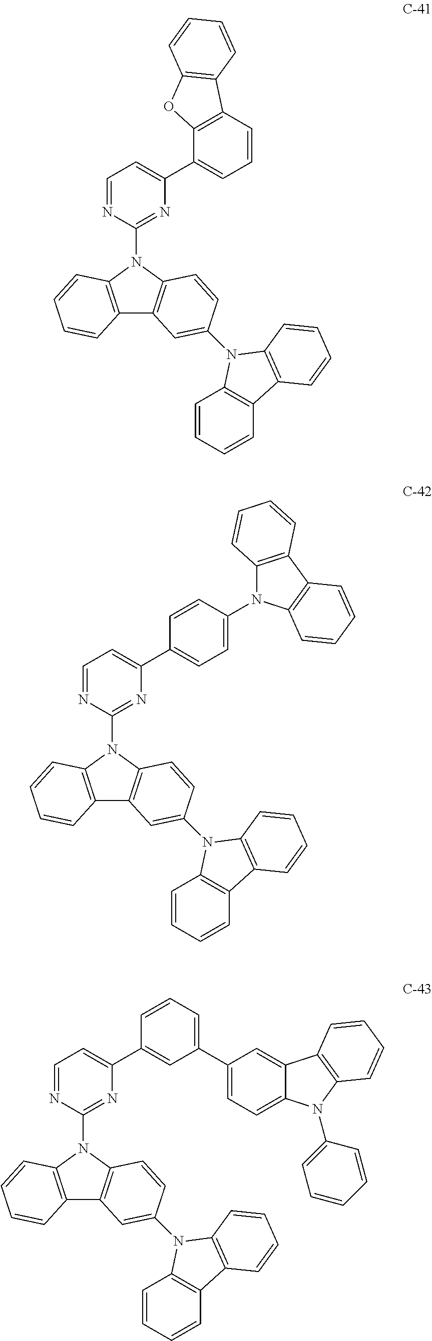 [SMILES: c1ccc2c(c1)oc1c(-c3ccnc(-n4c5ccccc5c5cc(-n6c7ccccc7c7ccccc76)ccc54)n3)cccc12 ; c1ccc2c(c1)c1ccccc1n2-c1ccc(-c2ccnc(-n3c4ccccc4c4cc(-n5c6ccccc6c6ccccc65)ccc43)n2)cc1 ; c1ccc(-n2c3ccccc3c3cc(-c4cccc(-c5ccnc(-n6c7ccccc7c7cc(-n8c9ccccc9c9ccccc98)ccc76)n5)c4)ccc32)cc1]

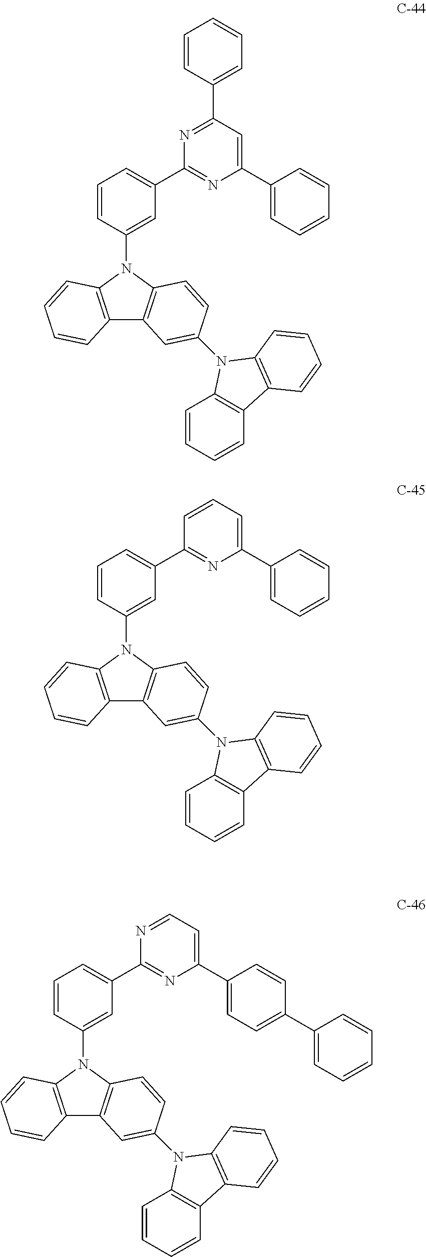

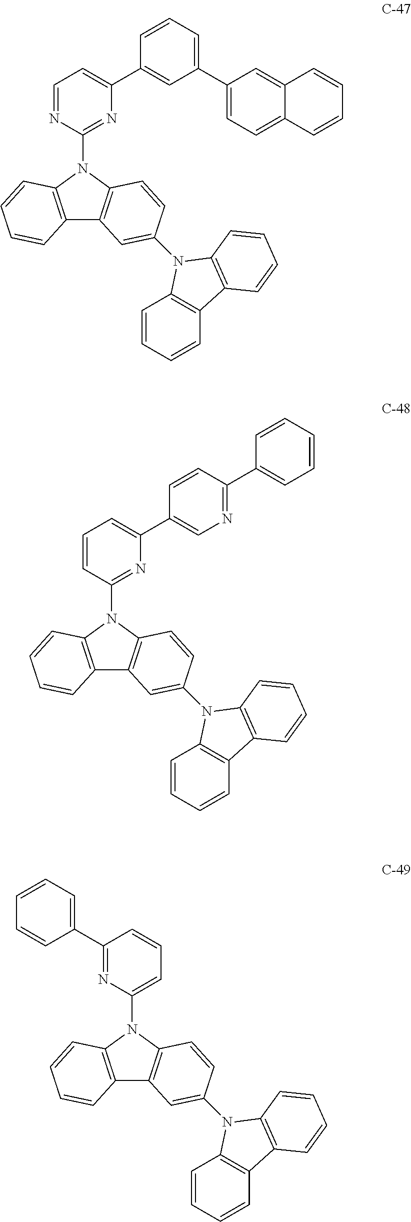 [SMILES: c1cc(-c2ccc3ccccc3c2)cc(-c2ccnc(-n3c4ccccc4c4cc(-n5c6ccccc6c6ccccc65)ccc43)n2)c1 ; c1ccc(-c2ccc(-c3cccc(-n4c5ccccc5c5cc(-n6c7ccccc7c7ccccc76)ccc54)n3)cn2)cc1 ; c1ccc(-c2cccc(-n3c4ccccc4c4cc(-n5c6ccccc6c6ccccc65)ccc43)n2)cc1]

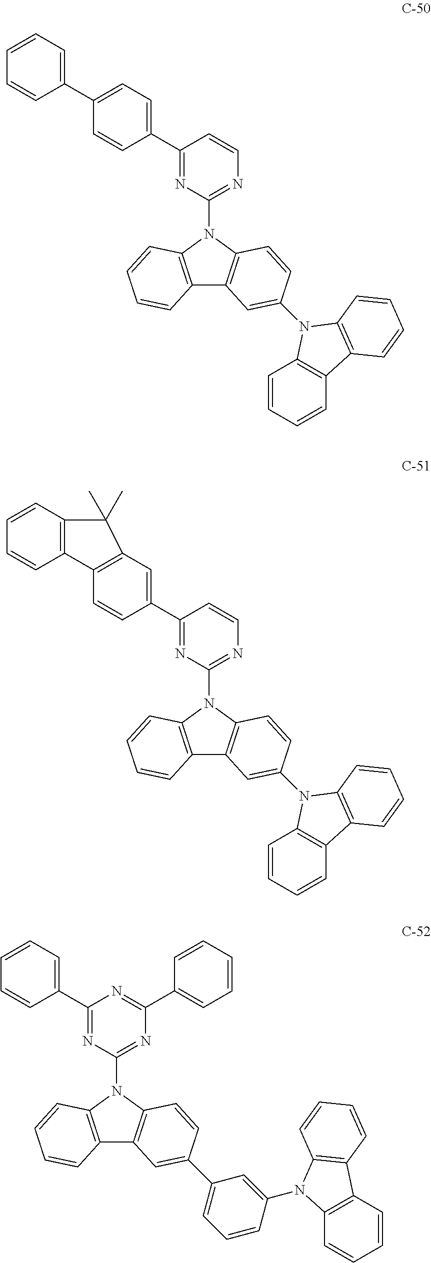 [SMILES: c1ccc(-c2ccc(-c3ccnc(-n4c5ccccc5c5cc(-n6c7ccccc7c7ccccc76)ccc54)n3)cc2)cc1 ; CC1(C)c2ccccc2-c2ccc(-c3ccnc(-n4c5ccccc5c5cc(-n6c7ccccc7c7ccccc76)ccc54)n3)cc21 ; c1ccc(-c2nc(-c3ccccc3)nc(-n3c4ccccc4c4cc(-c5cccc(-n6c7ccccc7c7ccccc76)c5)ccc43)n2)cc1]

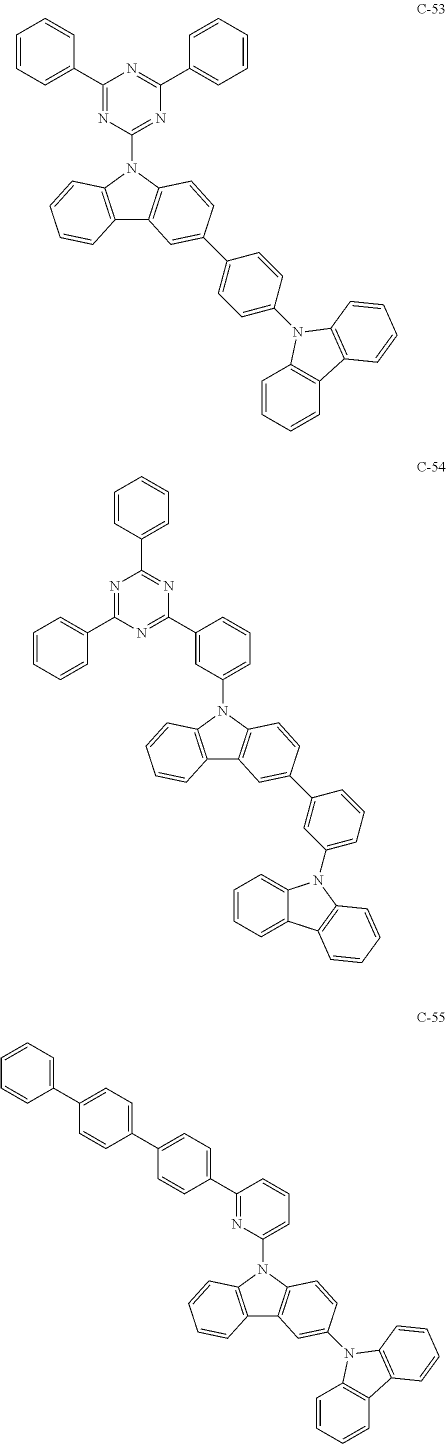 [SMILES: c1ccc(-c2nc(-c3ccccc3)nc(-n3c4ccccc4c4cc(-c5ccc(-n6c7ccccc7c7ccccc76)cc5)ccc43)n2)cc1 ; c1ccc(-c2nc(-c3ccccc3)nc(-c3cccc(-n4c5ccccc5c5cc(-c6cccc(-n7c8ccccc8c8ccccc87)c6)ccc54)c3)n2)cc1 ; c1ccc(-c2ccc(-c3ccc(-c4cccc(-n5c6ccccc6c6cc(-n7c8ccccc8c8ccccc87)ccc65)n4)cc3)cc2)cc1]

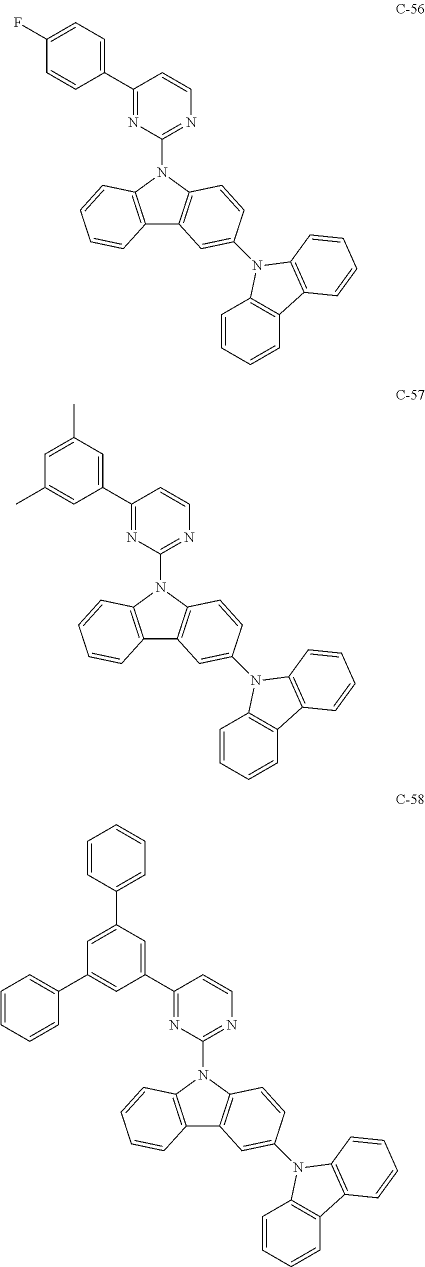 [SMILES: Fc1ccc(-c2ccnc(-n3c4ccccc4c4cc(-n5c6ccccc6c6ccccc65)ccc43)n2)cc1 ; Cc1cc(C)cc(-c2ccnc(-n3c4ccccc4c4cc(-n5c6ccccc6c6ccccc65)ccc43)n2)c1 ; c1ccc(-c2cc(-c3ccccc3)cc(-c3ccnc(-n4c5ccccc5c5cc(-n6c7ccccc7c7ccccc76)ccc54)n3)c2)cc1]

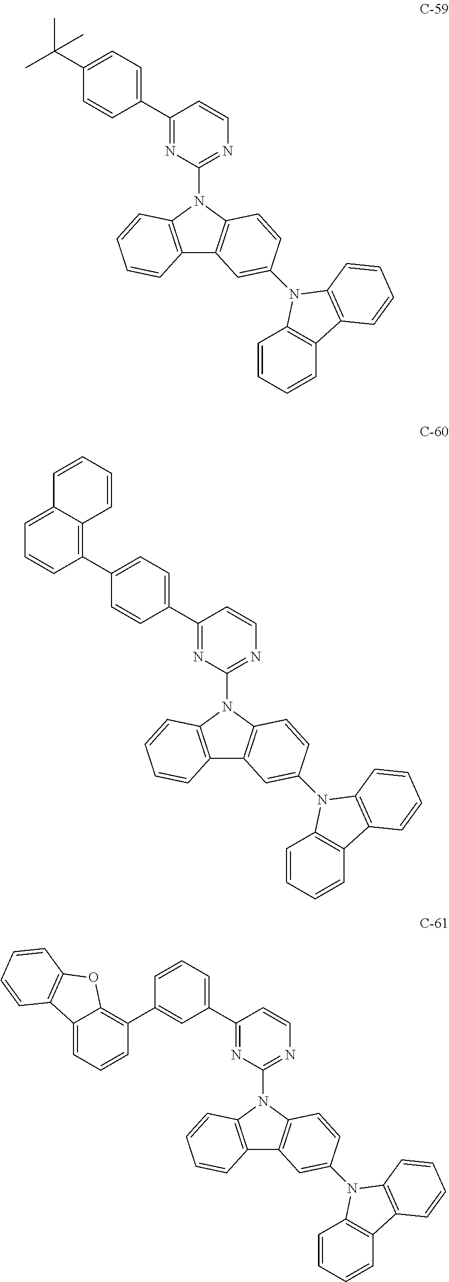 [SMILES: CC(C)(C)c1ccc(-c2ccnc(-n3c4ccccc4c4cc(-n5c6ccccc6c6ccccc65)ccc43)n2)cc1 ; c1ccc2c(-c3ccc(-c4ccnc(-n5c6ccccc6c6cc(-n7c8ccccc8c8ccccc87)ccc65)n4)cc3)cccc2c1 ; c1cc(-c2ccnc(-n3c4ccccc4c4cc(-n5c6ccccc6c6ccccc65)ccc43)n2)cc(-c2cccc3c2oc2ccccc23)c1]

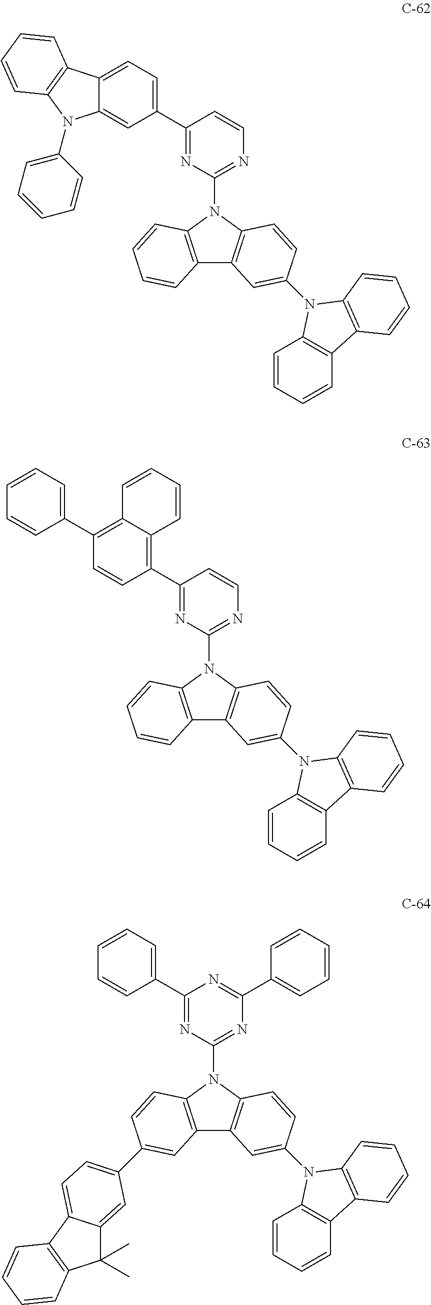 [SMILES: c1ccc(-n2c3ccccc3c3ccc(-c4ccnc(-n5c6ccccc6c6cc(-n7c8ccccc8c8ccccc87)ccc65)n4)cc32)cc1 ; c1ccc(-c2ccc(-c3ccnc(-n4c5ccccc5c5cc(-n6c7ccccc7c7ccccc76)ccc54)n3)c3ccccc23)cc1 ; CC1(C)c2ccccc2-c2ccc(-c3ccc4c(c3)c3cc(-n5c6ccccc6c6ccccc65)ccc3n4-c3nc(-c4ccccc4)nc(-c4ccccc4)n3)cc21]

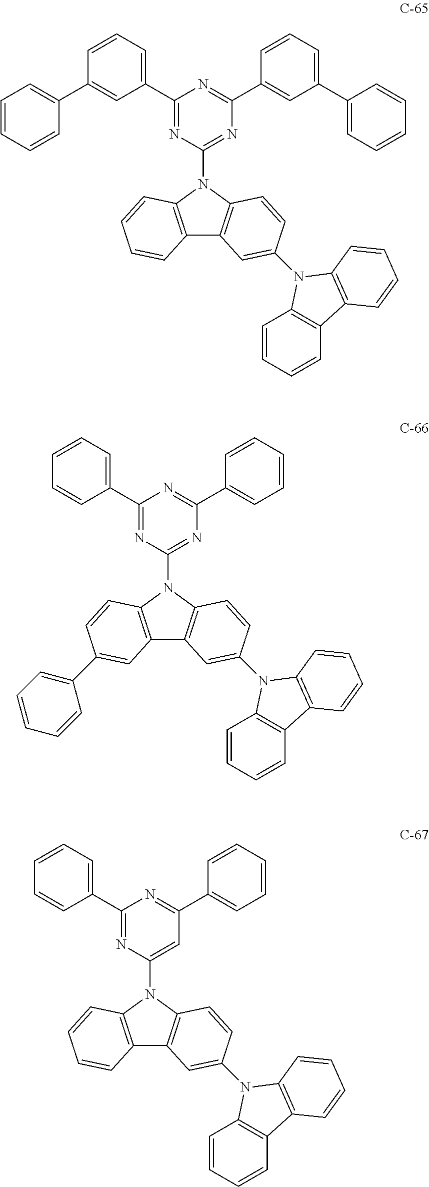 [SMILES: c1ccc(-c2cccc(-c3nc(-c4cccc(-c5ccccc5)c4)nc(-n4c5ccccc5c5cc(-n6c7ccccc7c7ccccc76)ccc54)n3)c2)cc1 ; c1ccc(-c2ccc3c(c2)c2cc(-n4c5ccccc5c5ccccc54)ccc2n3-c2nc(-c3ccccc3)nc(-c3ccccc3)n2)cc1 ; c1ccc(-c2cc(-n3c4ccccc4c4cc(-n5c6ccccc6c6ccccc65)ccc43)nc(-c3ccccc3)n2)cc1]

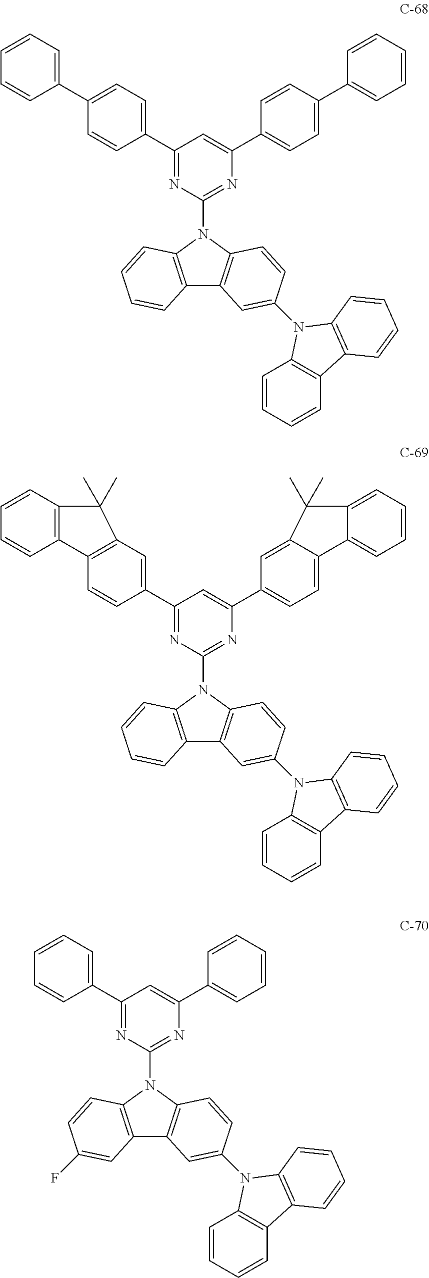 [SMILES: c1ccc(-c2ccc(-c3cc(-c4ccc(-c5ccccc5)cc4)nc(-n4c5ccccc5c5cc(-n6c7ccccc7c7ccccc76)ccc54)n3)cc2)cc1 ; CC1(C)c2ccccc2-c2ccc(-c3cc(-c4ccc5c(c4)C(C)(C)c4ccccc4-5)nc(-n4c5ccccc5c5cc(-n6c7ccccc7c7ccccc76)ccc54)n3)cc21 ; Fc1ccc2c(c1)c1cc(-n3c4ccccc4c4ccccc43)ccc1n2-c1nc(-c2ccccc2)cc(-c2ccccc2)n1]

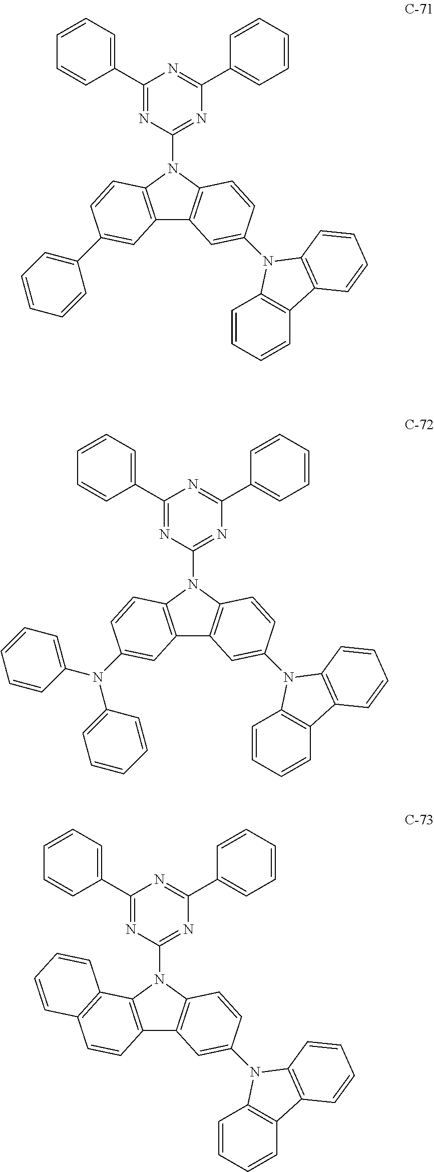 [SMILES: c1ccc(-c2ccc3c(c2)c2cc(-n4c5ccccc5c5ccccc54)ccc2n3-c2nc(-c3ccccc3)nc(-c3ccccc3)n2)cc1 ; c1ccc(-c2nc(-c3ccccc3)nc(-n3c4ccc(N(c5ccccc5)c5ccccc5)cc4c4cc(-n5c6ccccc6c6ccccc65)ccc43)n2)cc1 ; c1ccc(-c2nc(-c3ccccc3)nc(-n3c4ccc(-n5c6ccccc6c6ccccc65)cc4c4ccc5ccccc5c43)n2)cc1]

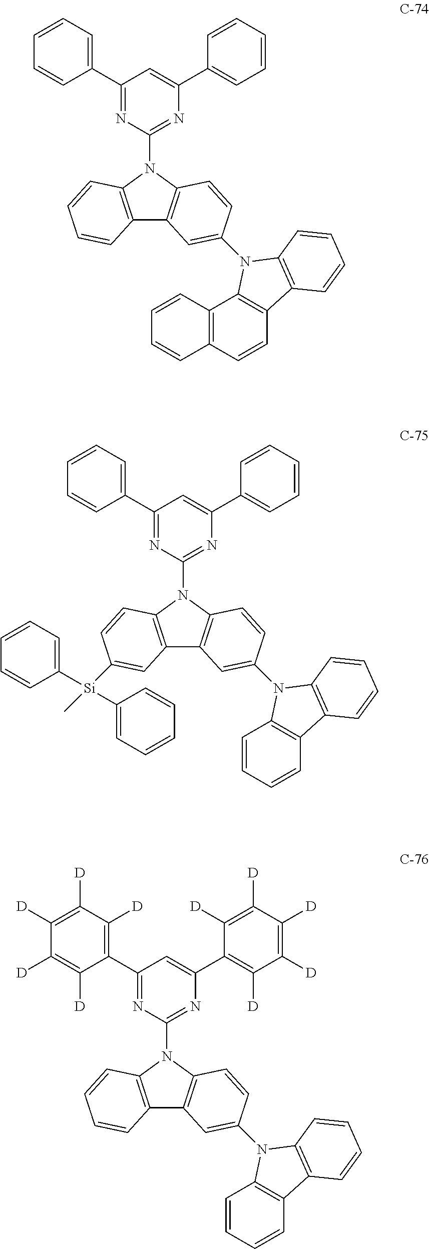 [SMILES: c1ccc(-c2cc(-c3ccccc3)nc(-n3c4ccccc4c4cc(-n5c6ccccc6c6ccc7ccccc7c65)ccc43)n2)cc1 ; C[Si](c1ccccc1)(c1ccccc1)c1ccc2c(c1)c1cc(-n3c4ccccc4c4ccccc43)ccc1n2-c1nc(-c2ccccc2)cc(-c2ccccc2)n1 ; [2H]c1c([2H])c([2H])c(-c2cc(-c3c([2H])c([2H])c([2H])c([2H])c3[2H])nc(-n3c4ccccc4c4cc(-n5c6ccccc6c6ccccc65)ccc43)n2)c([2H])c1[2H]]

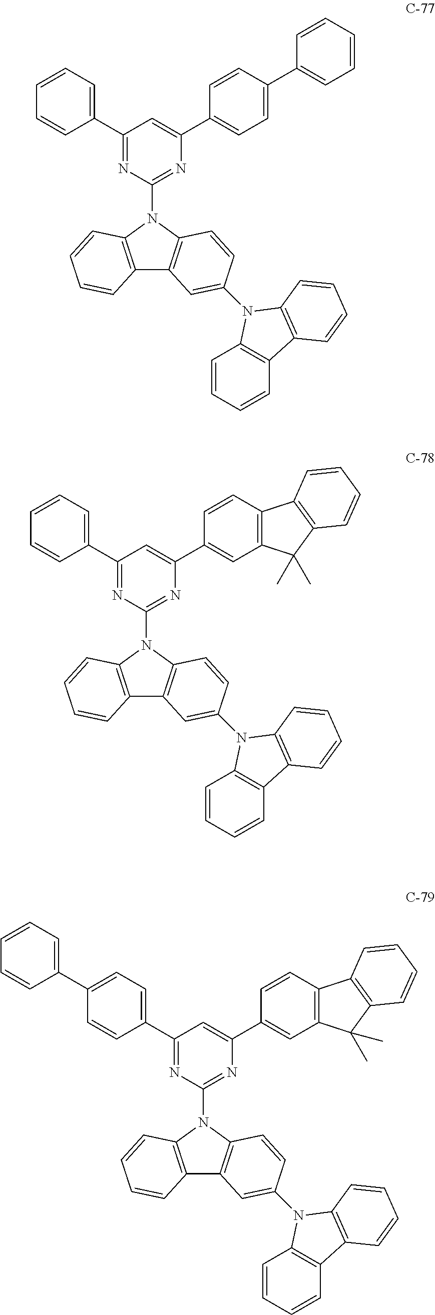 [SMILES: c1ccc(-c2ccc(-c3cc(-c4ccccc4)nc(-n4c5ccccc5c5cc(-n6c7ccccc7c7ccccc76)ccc54)n3)cc2)cc1 ; CC1(C)c2ccccc2-c2ccc(-c3cc(-c4ccccc4)nc(-n4c5ccccc5c5cc(-n6c7ccccc7c7ccccc76)ccc54)n3)cc21 ; CC1(C)c2ccccc2-c2ccc(-c3cc(-c4ccc(-c5ccccc5)cc4)nc(-n4c5ccccc5c5cc(-n6c7ccccc7c7ccccc76)ccc54)n3)cc21]

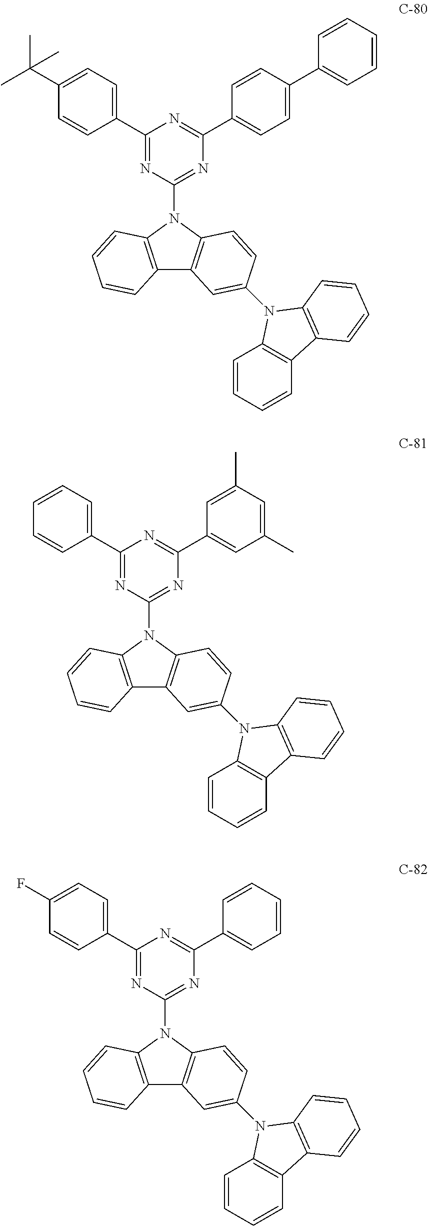 [SMILES: CC(C)(C)c1ccc(-c2nc(-c3ccc(-c4ccccc4)cc3)nc(-n3c4ccccc4c4cc(-n5c6ccccc6c6ccccc65)ccc43)n2)cc1 ; Cc1cc(C)cc(-c2nc(-c3ccccc3)nc(-n3c4ccccc4c4cc(-n5c6ccccc6c6ccccc65)ccc43)n2)c1 ; Fc1ccc(-c2nc(-c3ccccc3)nc(-n3c4ccccc4c4cc(-n5c6ccccc6c6ccccc65)ccc43)n2)cc1]

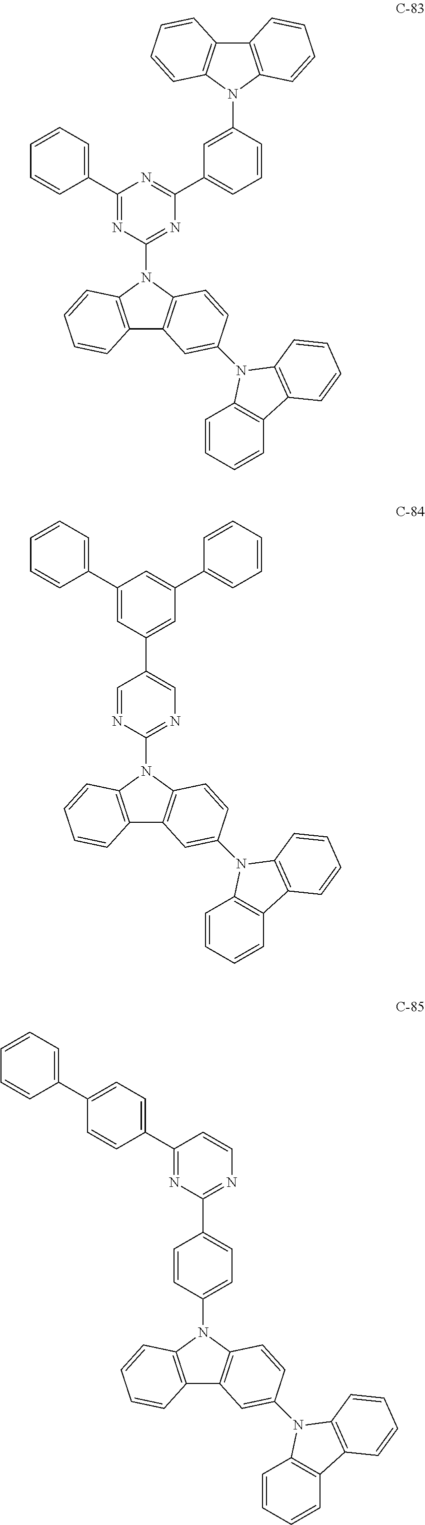 [SMILES: c1ccc(-c2nc(-c3cccc(-n4c5ccccc5c5ccccc54)c3)nc(-n3c4ccccc4c4cc(-n5c6ccccc6c6ccccc65)ccc43)n2)cc1 ; c1ccc(-c2cc(-c3ccccc3)cc(-c3cnc(-n4c5ccccc5c5cc(-n6c7ccccc7c7ccccc76)ccc54)nc3)c2)cc1 ; c1ccc(-c2ccc(-c3ccnc(-c4ccc(-n5c6ccccc6c6cc(-n7c8ccccc8c8ccccc87)ccc65)cc4)n3)cc2)cc1]

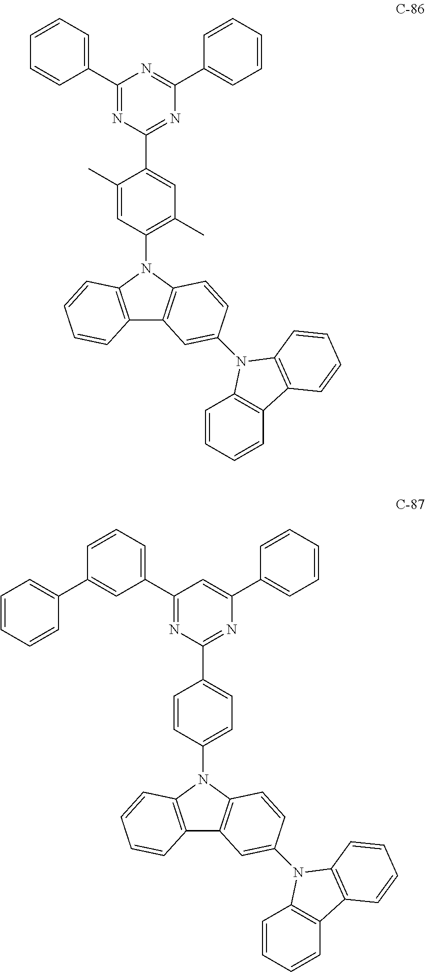 [SMILES: Cc1cc(-n2c3ccccc3c3cc(-n4c5ccccc5c5ccccc54)ccc32)c(C)cc1-c1nc(-c2ccccc2)nc(-c2ccccc2)n1 ; c1ccc(-c2cccc(-c3cc(-c4ccccc4)nc(-c4ccc(-n5c6ccccc6c6cc(-n7c8ccccc8c8ccccc87)ccc65)cc4)n3)c2)cc1]

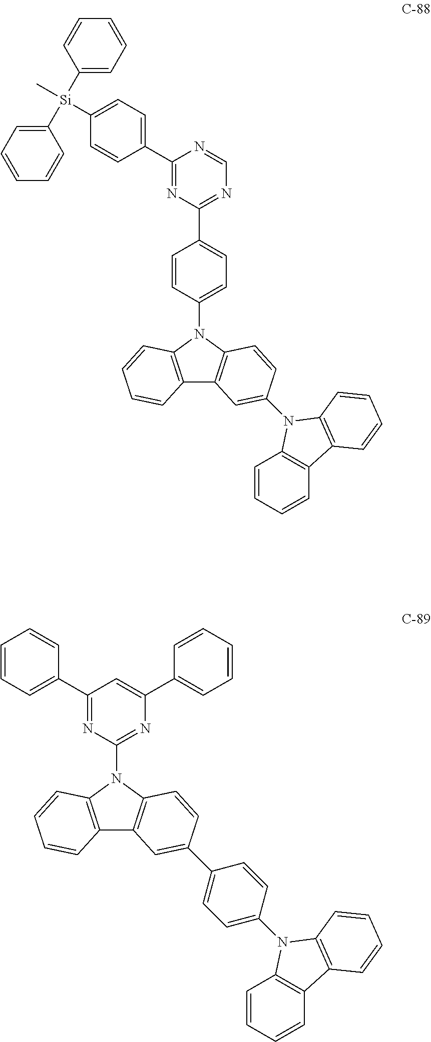 [SMILES: C[Si](c1ccccc1)(c1ccccc1)c1ccc(-c2ncnc(-c3ccc(-n4c5ccccc5c5cc(-n6c7ccccc7c7ccccc76)ccc54)cc3)n2)cc1 ; c1ccc(-c2cc(-c3ccccc3)nc(-n3c4ccccc4c4cc(-c5ccc(-n6c7ccccc7c7ccccc76)cc5)ccc43)n2)cc1]

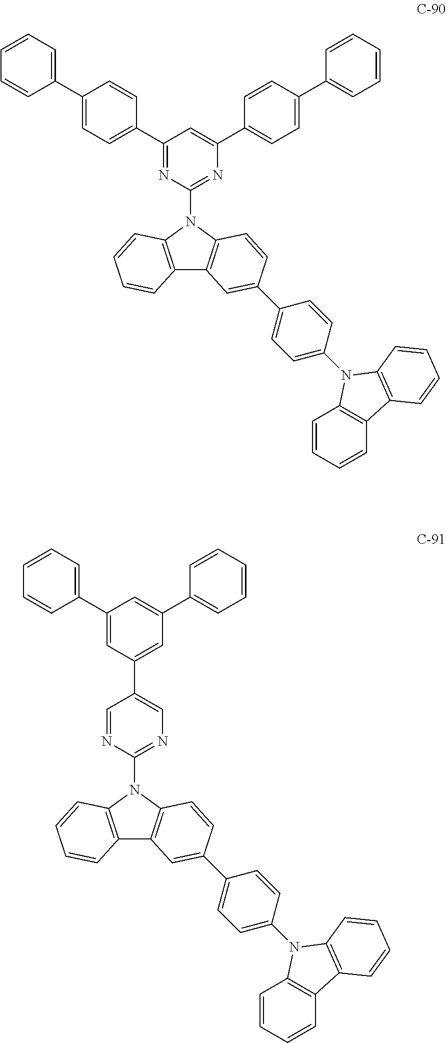 [SMILES: c1ccc(-c2ccc(-c3cc(-c4ccc(-c5ccccc5)cc4)nc(-n4c5ccccc5c5cc(-c6ccc(-n7c8ccccc8c8ccccc87)cc6)ccc54)n3)cc2)cc1 ; c1ccc(-c2cc(-c3ccccc3)cc(-c3cnc(-n4c5ccccc5c5cc(-c6ccc(-n7c8ccccc8c8ccccc87)cc6)ccc54)nc3)c2)cc1]

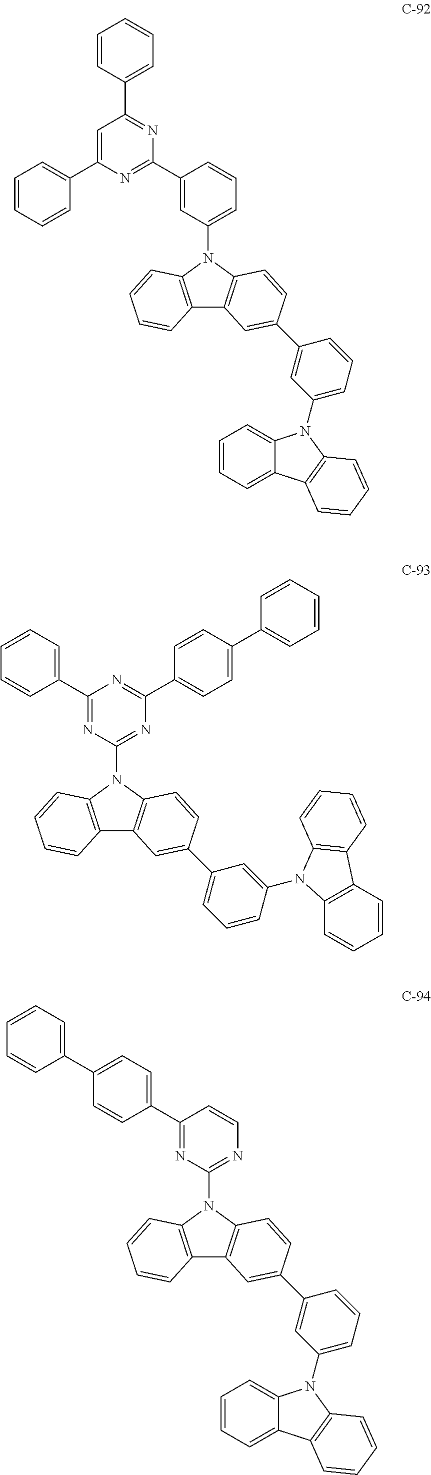 [SMILES: c1ccc(-c2cc(-c3ccccc3)nc(-c3cccc(-n4c5ccccc5c5cc(-c6cccc(-n7c8ccccc8c8ccccc87)c6)ccc54)c3)n2)cc1 ; c1ccc(-c2ccc(-c3nc(-c4ccccc4)nc(-n4c5ccccc5c5cc(-c6cccc(-n7c8ccccc8c8ccccc87)c6)ccc54)n3)cc2)cc1 ; c1ccc(-c2ccc(-c3ccnc(-n4c5ccccc5c5cc(-c6cccc(-n7c8ccccc8c8ccccc87)c6)ccc54)n3)cc2)cc1]

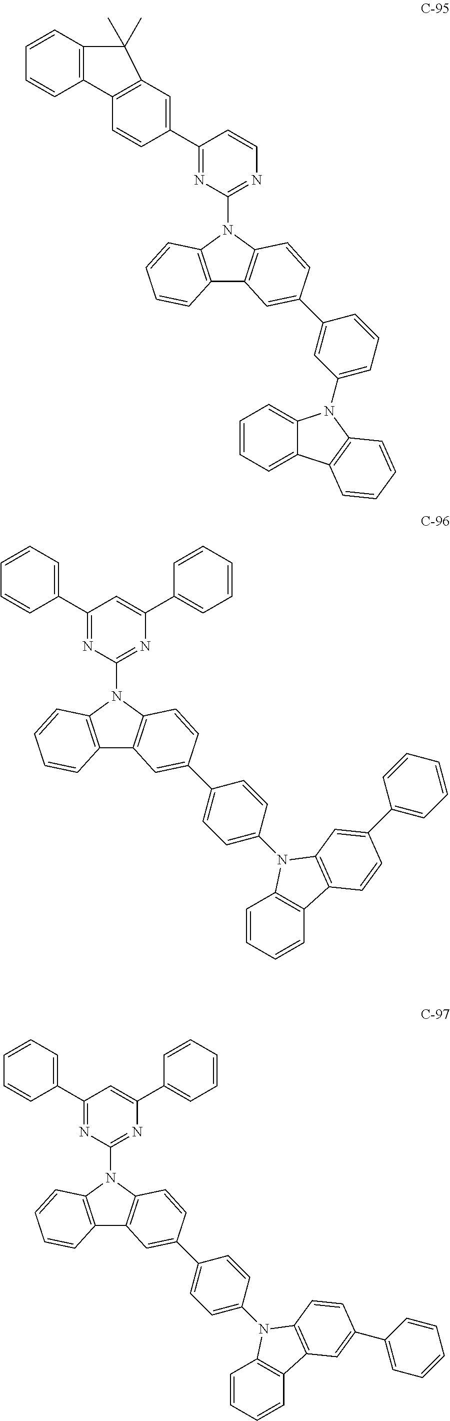 [SMILES: CC1(C)c2ccccc2-c2ccc(-c3ccnc(-n4c5ccccc5c5cc(-c6cccc(-n7c8ccccc8c8ccccc87)c6)ccc54)n3)cc21 ; c1ccc(-c2ccc3c4ccccc4n(-c4ccc(-c5ccc6c(c5)c5ccccc5n6-c5nc(-c6ccccc6)cc(-c6ccccc6)n5)cc4)c3c2)cc1 ; c1ccc(-c2ccc3c(c2)c2ccccc2n3-c2ccc(-c3ccc4c(c3)c3ccccc3n4-c3nc(-c4ccccc4)cc(-c4ccccc4)n3)cc2)cc1]

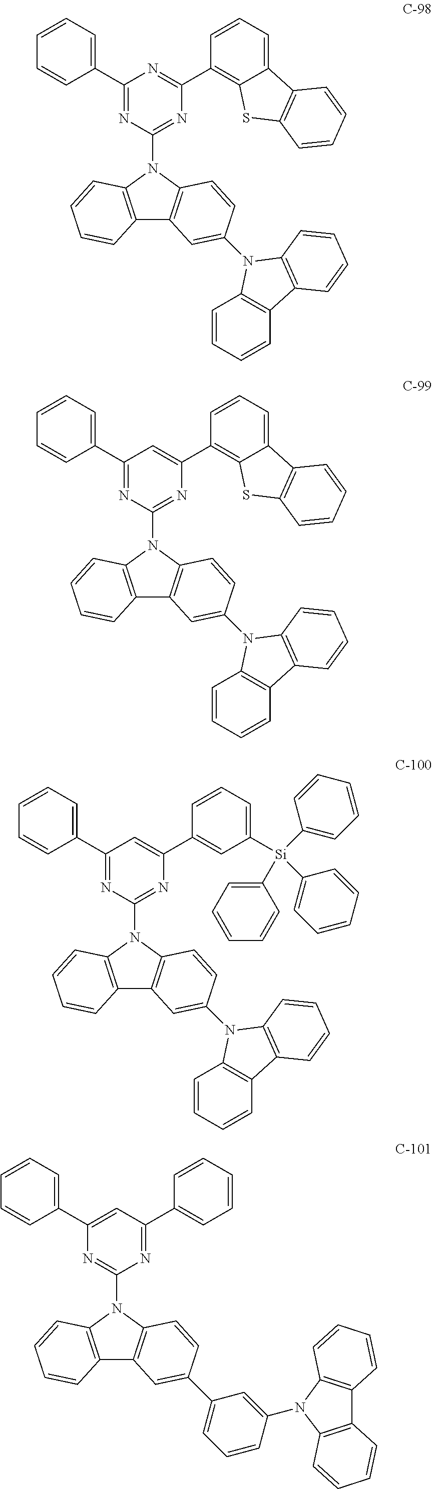

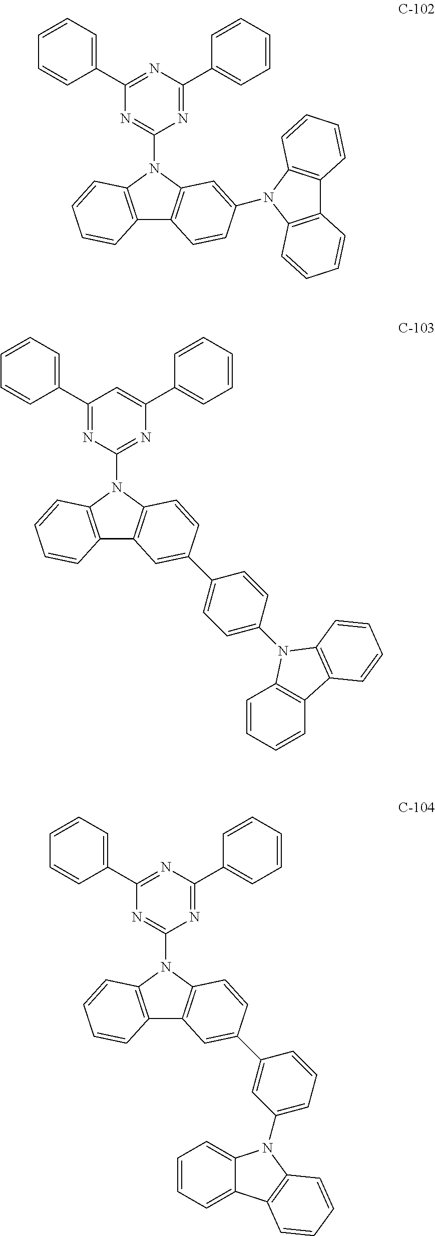 [SMILES: c1ccc(-c2nc(-c3ccccc3)nc(-n3c4ccccc4c4ccc(-n5c6ccccc6c6ccccc65)cc43)n2)cc1 ; c1ccc(-c2cc(-c3ccccc3)nc(-n3c4ccccc4c4cc(-c5ccc(-n6c7ccccc7c7ccccc76)cc5)ccc43)n2)cc1 ; c1ccc(-c2nc(-c3ccccc3)nc(-n3c4ccccc4c4cc(-c5cccc(-n6c7ccccc7c7ccccc76)c5)ccc43)n2)cc1]

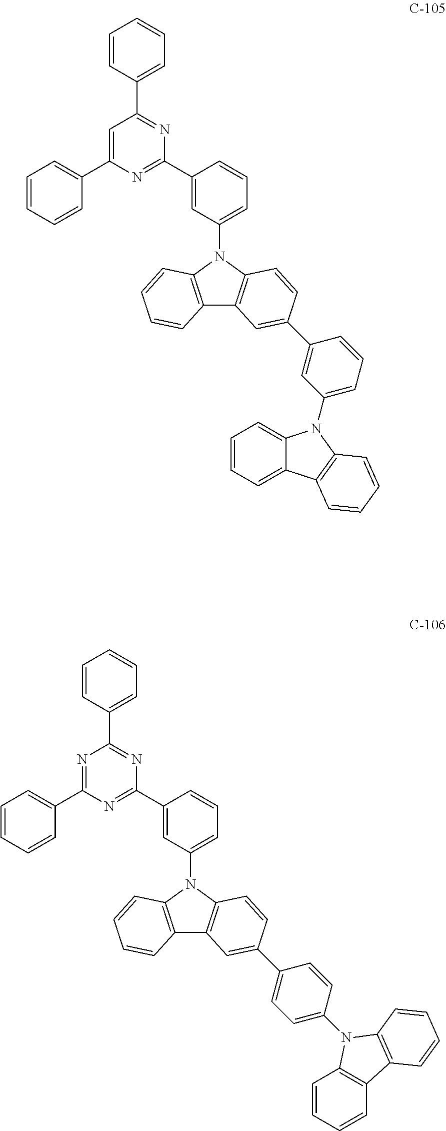 [SMILES: c1ccc(-c2cc(-c3ccccc3)nc(-c3cccc(-n4c5ccccc5c5cc(-c6cccc(-n7c8ccccc8c8ccccc87)c6)ccc54)c3)n2)cc1 ; c1ccc(-c2nc(-c3ccccc3)nc(-c3cccc(-n4c5ccccc5c5cc(-c6ccc(-n7c8ccccc8c8ccccc87)cc6)ccc54)c3)n2)cc1]

7. An organic electroluminescent device comprising the organic electroluminescent compound according to claim 1.
